SEQUENCE LISTING (54 sequences)

SEQ ID NO: 1 - DNA, 18 bp, Artificial Sequence
  Synthetic
taatacgactcactatag

SEQ ID NO: 2 - DNA, 2610 bp, Artificial Sequence
  Synthetic
atgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaa

SEQ ID NO: 3 - DNA, 2610 bp, Artificial Sequence
  Synthetic
atgaaacatcaccatcaccatcaccccatgagcgattacgacatccccactactgagaatctttattttcagggcgccgacgccaacgtagtgagctcgtccacgattgctacatacatcgacgcactggctaaaaacgcgagtgaattagagcaacgttcaaccgcctatgaaatcaacaacgaacttgagctcgtctttattaagcctccgctaatcaccctgactaacgttgttaatatatctaccatccaggaaagcttcattcgcttcactgttactaacaaagaaggcgtaaaaatcaggactaaaatcccattgtctaaggtgcacgggctggatgtgaaaaacgttcagctggttgacgctattgacaacatcgtatgggaaaagaaatccctcgtaaccgaaaaccgtctgcataaagaatgtctgctgcgtctgagcacggaggaacgacacatctttctggattacaaaaaatatggtagttctattcgtctggagctggtgaacctgatccaggcaaagaccaaaaatttcacaattgacttcaaactaaaatactttctgggctccggtgcgcagagcaaatcttccctgttgcatgctatcaaccacccgaaaagccgcccgaatacttctctggaaatcgagttcaccccccgcgataacgaaactgtcccatacgatgagcttattaaggaactgaccacgctgtcccgtcacatttttatggcgagcccggaaaacgttatattatcgccgcctatcaacgctccgatcaagaccttcatgttgccgaaacaagacatcgtcggtctggatctggagaacctgtacgcagttactaaaaccgacggcatccccatcactatcagagtaacgtcaaacggattgtattgctatttcacccatctgggttacattattcgttacccggtgaaacgcatcatagattctgaagttgttgttttcggcgaagccgtaaaggacaaaaactggaccgtctatctgatcaagctaatcgaaccggttaatgctatcaacgatcggctggaagaatcgaaatacgtagaatctaaactggtggatatttgcgaccgtattgtctttaaatcgaaaaagtacgagggtcctttcactactactagcgaagtcgtggacatgctctctacgtacctgccgaaacagcctgagggcgttatcctgttctatagcaaaggtccgaaatccaacatcgattttaagattaaaaaggaaaacaccattgatcagacggctaatgtagttttccggtacatgtctagcgagccgatcatctttggcgaatcttctatctttgtagaatataaaaagttcagcaacgacaaaggattcccaaaagaatacgggtccgggaaaatcgtcttatacaacggtgttaactacttgaacaacatctattgcctggaatatatcaatactcacaatgaagttggtattaaatcagtggttgttccgataaaattcatcgcggaatttctggtcaatggcgaaatcctgaaaccccgcattgataagaccatgaaatacataaactccgaagactactacggtaaccagcataacatcatcgtggaacacctgagagatcagagtatcaaaatcggcgacattttcaatgaggacaagttaagcgacgtgggccatcaatacgcaaacaacgacaaattccgtctgaacccggaggtttcctatttcaccaacaaacgtacccgaggtccgcttggcatcctctccaattacgtaaaaaccctgctgatttctatgtattgttcaaaaacgttcctggatgacagcaacaaaaggaaggtactggctatcgatttcggtaacggcgcggatctggaaaagtacttttacggtgaaatcgctctgttagtcgcaactgatccggacgccgacgcaattgctcgcggaaatgaacgttacaacaaactgaactccggtattaaaacaaagtattataaattcgactatatccaggagactatccgctctgatactttcgtgagcagcgtgcgtgaggttttttactttggtaaattcaacattattgactggcagtttgcgatccactacagctttcacccgcgtcactatgcgaccgttatgaataacctatcggaactcacggctagcggcggcaaagtgctgattactactatggacggtgacaaactgtctaagctgaccgataagaaaaccttcatcatccacaaaaacttgccaagttctgagaactatatgtctgttgaaaaaattgcggacgaccgcatcgtcgtttacaacccatctaccatgtccacccctatgacagagtacatcatcaaaaagaacgacatagttcgtgttttcaacgaatacggcttcgtactggtagataacgtcgattttgctaccattatcgagcgttcgaaaaaattcattaacggtgcttccactatggaagatcgtccgtccactcgtaacttttttgaattaaaccgtggcgcaatcaaatgcgaagggctggatgtggaagacctcctgtcttactacgttgtatacgtcttctctaaacgctaa

SEQ ID NO: 4 - DNA, 957 bp, Artificial Sequence
  Synthetic
atggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataa

SEQ ID NO: 5 - DNA, 957 bp, Artificial Sequence
  Synthetic
atggatgagatcgttaagaacattcgtgaaggtacgcatgtgcttttgccattttacgaaactctcccggaactgaatctgtccttaggcaaaagccctctaccctctctggagtatggggccaactacttcctgcaaatctcacgcgtcaacgacctgaatcgaatgccgaccgacatgctgaaactgttcactcacgatataatgctgccggaaagtgatctggacaaagtatatgaaatcctgaaaatcaacagcgttaagtactacggacggtcgaccaaagcggacgctgttgtagcagatctgtctgctcgcaacaaactctttaaacgtgaacgtgacgctattaagtccaacaaccacctgacagagaacaatctctatatctctgactacaaaatgttgactttcgatgtgttccgtccgctgtttgatttcgtgaacgaaaaatattgcattatcaaactgccgaccctgttcggccgtggtgttattgacaccatgcgcatctactgtagcctcttcaagaatgtcagactactgaaatgcgtgtccgatagctggctgaaagacagcgcaatcatggtagcctcagacgtttgcaaaaagaacctggatctgtttatgtcccatgttaaatccgttactaagtctagctcgtggaaagatgttaacagcgtacagttttctattttgaacaaccctgttgacacggaatttatcaacaaattcctggagttctctaaccgtgtatacgaagcgctgtattacgtgcactccttactgtactcttctatgaccagcgatagtaagtctatcgaaaataaacaccagcgccgtctggtaaaactgctccttgggagcgcttggagccacccgcagttcgaaaaaggtggaggttctggcggtggatcgggaggttcagcgtggagccacccgcagttcgagaaataa

SEQ ID NO: 6 - protein, 869 aa, Artificial Sequence
  Synthetic
MKHHHHHHPMSDYDIPTTENLYFQGADANVVSSSTIATYIDALAKNASELEQRSTAYEINNELELVFIKPPLITLTNVVNISTIQESFIRFTVTNKEGVKIRTKIPLSKVHGLDVKNVQLVDAIDNIVWEKKSLVTENRLHKECLLRLSTEERHIFLDYKKYGSSIRLELVNLIQAKTKNFTIDFKLKYFLGSGAQSKSSLLHAINHPKSRPNTSLEIEFTPRDNETVPYDELIKELTTLSRHIFMASPENVILSPPINAPIKTFMLPKQDIVGLDLENLYAVTKTDGIPITIRVTSNGLYCYFTHLGYIIRYPVKRIIDSEVVVFGEAVKDKNWTVYLIKLIEPVNAINDRLEESKYVESKLVDICDRIVFKSKKYEGPFTTTSEVVDMLSTYLPKQPEGVILFYSKGPKSNIDFKIKKENTIDQTANVVFRYMSSEPIIFGESSIFVEYKKFSNDKGFPKEYGSGKIVLYNGVNYLNNIYCLEYINTHNEVGIKSVVVPIKFIAEFLVNGEILKPRIDKTMKYINSEDYYGNQHNIIVEHLRDQSIKIGDIFNEDKLSDVGHQYANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTRNFFELNRGAIKCEGLDVEDLLSYYVVYVFSKR

SEQ ID NO: 7 - protein, 318 aa, Artificial Sequence
  Synthetic
MDEIVKNIREGTHVLLPFYETLPELNLSLGKSPLPSLEYGANYFLQISRVNDLNRMPTDMLKLFTHDIMLPESDLDKVYEILKINSVKYYGRSTKADAVVADLSARNKLFKRERDAIKSNNHLTENNLYISDYKMLTFDVFRPLFDFVNEKYCIIKLPTLFGRGVIDTMRIYCSLFKNVRLLKCVSDSWLKDSAIMVASDVCKKNLDLFMSHVKSVTKSSSWKDVNSVQFSILNNPVDTEFINKFLEFSNRVYEALYYVHSLLYSSMTSDSKSIENKHQRRLVKLLLGSAWSHPQFEKGGGSGGGSGGSAWSHPQFEK

SEQ ID NO: 8 - DNA, 56 bp, Artificial Sequence
  Synthetic
tgttgacaattaatcatcggctcgtataatgtgtggaattgtgagcgctcacaatt

SEQ ID NO: 9 - DNA, 118 bp, Artificial Sequence
  Synthetic
aattgtgagcggataacaattacgagcttcatgcacagtgaaatcatgaaaaatttatttgctttgtgagcggataacaattataatatgtggaattgtgagcgctcacaattccaca

SEQ ID NO: 10 - DNA, 68 bp, Artificial Sequence
  Synthetic
gcgaaaaatcaataaggaggcaacaagatgtgcgaaaaacatcttaatcatgcacaggagactttcta

SEQ ID NO: 11 - DNA, 80 bp, Artificial Sequence
  Synthetic
gtcaataaaggcatataaaaggaggttaataacatgaaagttaaagtaaaacatcttaatcatgctaaggaggttttcta

SEQ ID NO: 12 - DNA, 68 bp, Artificial Sequence
  Synthetic
gcgaaaaatcaataaggaggcaacaagatgtgcgaaaaacatcttaatcatgcgccggaggttttcta

SEQ ID NO: 13 - DNA, 68 bp, Artificial Sequence
  Synthetic
gcgaaaaatcaataaggaggcaacaagatgtgcgaaaaacatcttaatcatgcggaggatcgtttcta

SEQ ID NO: 14 - DNA, 80 bp, Artificial Sequence
  Synthetic
gtcaataaaggcatataaaaggaggttaataacatgaaagttaaagtaaaacatcttaatcatgcgggggagtgtttcta

SEQ ID NO: 15 - DNA, 68 bp, Artificial Sequence
  Synthetic
gcgaaaaatcaataaggaggcaacaagatgtgcgaaaaacatcttaatcatgcggtggagggtttcta

SEQ ID NO: 16 - DNA, 80 bp, Artificial
  Synthetic
gtcaataaaggcatataaaaggaggttaataacatgaaagttaaagtaaaacatcttaatcatgcgggggagtctttcta

SEQ ID NO: 17 - DNA, 68 bp, Artificial Sequence
  Synthetic
gcgaaaaatcaataaggaggcaacaagatgtgcgaaaaacatcttaatcatgcgacggagcgtttcta

SEQ ID NO: 18 - DNA, 113 bp, Artificial Sequence
  Synthetic
ccggcttatcggtcagtttcacctgatttacgtaaaaacccgcttcggcgggtttttgcttttggaggggcagaaagatgaatgactgtccacgacgctatacccaaaagaaa

SEQ ID NO: 19 - DNA, 129 bp, Artificial Sequence
  Synthetic
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 20 - DNA, 44 bp, Artificial Sequence
  Synthetic
ataaccccttggggcctctaaacgggtcttgaggggttttttgc

SEQ ID NO: 21 - DNA, 4163 bp, Artificial Sequence
  Synthetic
aattgtgagcggataacaattacgagcttcatgcacagtgaaatcatgaaaaatttatttgctttgtgagcggataacaattataatatgtggaattgtgagcgctcacaattccacagcgaaaaatcaataaggaggcaacaagatgtgcgaaaaacatcttaatcatgcacaggagactttctaatgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaaccggcttatcggtcagtttcacctgatttacgtaaaaacccgcttcggcgggtttttgcttttggaggggcagaaagatgaatgactgtccacgacgctatacccaaaagaaatgttgacaattaatcatcggctcgtataatgtgtggaattgtgagcgctcacaattgcgaaaaatcaataaggaggcaacaagatgtgcgaaaaacatcttaatcatgcgccggaggttttctaatggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttataataaccccttggggcctctaaacgggtcttgaggggttttttgc

SEQ ID NO: 22 - DNA, 3944 bp, Artificial Sequence
  Synthetic
tgttgacaattaatcatcggctcgtataatgtgtggaattgtgagcgctcacaattgcgaaaaatcaataaggaggcaacaagatgtgcgaaaaacatcttaatcatgcggaggatcgtttctaatgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaagtcaataaaggcatataaaaggaggttaataacatgaaagttaaagtaaaacatcttaatcatgcgggggagtgtttctaatggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttataataaccccttggggcctctaaacgggtcttgaggggttttttgc

SEQ ID NO: 23 - DNA, 4163 bp, Artificial Sequence
  Synthetic
aattgtgagcggataacaattacgagcttcatgcacagtgaaatcatgaaaaatttatttgctttgtgagcggataacaattataatatgtggaattgtgagcgctcacaattccacagcgaaaaatcaataaggaggcaacaagatgtgcgaaaaacatcttaatcatgcgacggagcgtttctaatgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaaccggcttatcggtcagtttcacctgatttacgtaaaaacccgcttcggcgggtttttgcttttggaggggcagaaagatgaatgactgtccacgacgctatacccaaaagaaatgttgacaattaatcatcggctcgtataatgtgtggaattgtgagcgctcacaattgcgaaaaatcaataaggaggcaacaagatgtgcgaaaaacatcttaatcatgcgccggaggttttctaatggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttataataaccccttggggcctctaaacgggtcttgaggggttttttgc

SEQ ID NO: 24 - DNA, 4175 bp, Artificial Sequence
  Synthetic
aattgtgagcggataacaattacgagcttcatgcacagtgaaatcatgaaaaatttatttgctttgtgagcggataacaattataatatgtggaattgtgagcgctcacaattccacagcgaaaaatcaataaggaggcaacaagatgtgcgaaaaacatcttaatcatgcgacggagcgtttctaatgaaacatcaccatcaccatcaccccatgagcgattacgacatccccactactgagaatctttattttcagggcgccgacgccaacgtagtgagctcgtccacgattgctacatacatcgacgcactggctaaaaacgcgagtgaattagagcaacgttcaaccgcctatgaaatcaacaacgaacttgagctcgtctttattaagcctccgctaatcaccctgactaacgttgttaatatatctaccatccaggaaagcttcattcgcttcactgttactaacaaagaaggcgtaaaaatcaggactaaaatcccattgtctaaggtgcacgggctggatgtgaaaaacgttcagctggttgacgctattgacaacatcgtatgggaaaagaaatccctcgtaaccgaaaaccgtctgcataaagaatgtctgctgcgtctgagcacggaggaacgacacatctttctggattacaaaaaatatggtagttctattcgtctggagctggtgaacctgatccaggcaaagaccaaaaatttcacaattgacttcaaactaaaatactttctgggctccggtgcgcagagcaaatcttccctgttgcatgctatcaaccacccgaaaagccgcccgaatacttctctggaaatcgagttcaccccccgcgataacgaaactgtcccatacgatgagcttattaaggaactgaccacgctgtcccgtcacatttttatggcgagcccggaaaacgttatattatcgccgcctatcaacgctccgatcaagaccttcatgttgccgaaacaagacatcgtcggtctggatctggagaacctgtacgcagttactaaaaccgacggcatccccatcactatcagagtaacgtcaaacggattgtattgctatttcacccatctgggttacattattcgttacccggtgaaacgcatcatagattctgaagttgttgttttcggcgaagccgtaaaggacaaaaactggaccgtctatctgatcaagctaatcgaaccggttaatgctatcaacgatcggctggaagaatcgaaatacgtagaatctaaactggtggatatttgcgaccgtattgtctttaaatcgaaaaagtacgagggtcctttcactactactagcgaagtcgtggacatgctctctacgtacctgccgaaacagcctgagggcgttatcctgttctatagcaaaggtccgaaatccaacatcgattttaagattaaaaaggaaaacaccattgatcagacggctaatgtagttttccggtacatgtctagcgagccgatcatctttggcgaatcttctatctttgtagaatataaaaagttcagcaacgacaaaggattcccaaaagaatacgggtccgggaaaatcgtcttatacaacggtgttaactacttgaacaacatctattgcctggaatatatcaatactcacaatgaagttggtattaaatcagtggttgttccgataaaattcatcgcggaatttctggtcaatggcgaaatcctgaaaccccgcattgataagaccatgaaatacataaactccgaagactactacggtaaccagcataacatcatcgtggaacacctgagagatcagagtatcaaaatcggcgacattttcaatgaggacaagttaagcgacgtgggccatcaatacgcaaacaacgacaaattccgtctgaacccggaggtttcctatttcaccaacaaacgtacccgaggtccgcttggcatcctctccaattacgtaaaaaccctgctgatttctatgtattgttcaaaaacgttcctggatgacagcaacaaaaggaaggtactggctatcgatttcggtaacggcgcggatctggaaaagtacttttacggtgaaatcgctctgttagtcgcaactgatccggacgccgacgcaattgctcgcggaaatgaacgttacaacaaactgaactccggtattaaaacaaagtattataaattcgactatatccaggagactatccgctctgatactttcgtgagcagcgtgcgtgaggttttttactttggtaaattcaacattattgactggcagtttgcgatccactacagctttcacccgcgtcactatgcgaccgttatgaataacctatcggaactcacggctagcggcggcaaagtgctgattactactatggacggtgacaaactgtctaagctgaccgataagaaaaccttcatcatccacaaaaacttgccaagttctgagaactatatgtctgttgaaaaaattgcggacgaccgcatcgtcgtttacaacccatctaccatgtccacccctatgacagagtacatcatcaaaaagaacgacatagttcgtgttttcaacgaatacggcttcgtactggtagataacgtcgattttgctaccattatcgagcgttcgaaaaaattcattaacggtgcttccactatggaagatcgtccgtccactcgtaacttttttgaattaaaccgtggcgcaatcaaatgcgaagggctggatgtggaagacctcctgtcttactacgttgtatacgtcttctctaaacgctaaccggcttatcggtcagtttcacctgatttacgtaaaaacccgcttcggcgggtttttgcttttggaggggcagaaagatgaatgactgtccacgacgctatacccaaaagaaatgttgacaattaatcatcggctcgtataatgtgtggaattgtgagcgctcacaattgtcaataaaggcatataaaaggaggttaataacatgaaagttaaagtaaaacatcttaatcatgcgggggagtctttctaatggatgagatcgttaagaacattcgtgaaggtacgcatgtgcttttgccattttacgaaactctcccggaactgaatctgtccttaggcaaaagccctctaccctctctggagtatggggccaactacttcctgcaaatctcacgcgtcaacgacctgaatcgaatgccgaccgacatgctgaaactgttcactcacgatataatgctgccggaaagtgatctggacaaagtatatgaaatcctgaaaatcaacagcgttaagtactacggacggtcgaccaaagcggacgctgttgtagcagatctgtctgctcgcaacaaactctttaaacgtgaacgtgacgctattaagtccaacaaccacctgacagagaacaatctctatatctctgactacaaaatgttgactttcgatgtgttccgtccgctgtttgatttcgtgaacgaaaaatattgcattatcaaactgccgaccctgttcggccgtggtgttattgacaccatgcgcatctactgtagcctcttcaagaatgtcagactactgaaatgcgtgtccgatagctggctgaaagacagcgcaatcatggtagcctcagacgtttgcaaaaagaacctggatctgtttatgtcccatgttaaatccgttactaagtctagctcgtggaaagatgttaacagcgtacagttttctattttgaacaaccctgttgacacggaatttatcaacaaattcctggagttctctaaccgtgtatacgaagcgctgtattacgtgcactccttactgtactcttctatgaccagcgatagtaagtctatcgaaaataaacaccagcgccgtctggtaaaactgctccttgggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttataataaccccttggggcctctaaacgggtcttgaggggttttttgc

SEQ ID NO: 25 - DNA, 4175 bp, Artificial Sequence
  Synthetic
aattgtgagcggataacaattacgagcttcatgcacagtgaaatcatgaaaaatttatttgctttgtgagcggataacaattataatatgtggaattgtgagcgctcacaattccacagcgaaaaatcaataaggaggcaacaagatgtgcgaaaaacatcttaatcatgcacaggagactttctaatgaaacatcaccatcaccatcaccccatgagcgattacgacatccccactactgagaatctttattttcagggcgccgacgccaacgtagtgagctcgtccacgattgctacatacatcgacgcactggctaaaaacgcgagtgaattagagcaacgttcaaccgcctatgaaatcaacaacgaacttgagctcgtctttattaagcctccgctaatcaccctgactaacgttgttaatatatctaccatccaggaaagcttcattcgcttcactgttactaacaaagaaggcgtaaaaatcaggactaaaatcccattgtctaaggtgcacgggctggatgtgaaaaacgttcagctggttgacgctattgacaacatcgtatgggaaaagaaatccctcgtaaccgaaaaccgtctgcataaagaatgtctgctgcgtctgagcacggaggaacgacacatctttctggattacaaaaaatatggtagttctattcgtctggagctggtgaacctgatccaggcaaagaccaaaaatttcacaattgacttcaaactaaaatactttctgggctccggtgcgcagagcaaatcttccctgttgcatgctatcaaccacccgaaaagccgcccgaatacttctctggaaatcgagttcaccccccgcgataacgaaactgtcccatacgatgagcttattaaggaactgaccacgctgtcccgtcacatttttatggcgagcccggaaaacgttatattatcgccgcctatcaacgctccgatcaagaccttcatgttgccgaaacaagacatcgtcggtctggatctggagaacctgtacgcagttactaaaaccgacggcatccccatcactatcagagtaacgtcaaacggattgtattgctatttcacccatctgggttacattattcgttacccggtgaaacgcatcatagattctgaagttgttgttttcggcgaagccgtaaaggacaaaaactggaccgtctatctgatcaagctaatcgaaccggttaatgctatcaacgatcggctggaagaatcgaaatacgtagaatctaaactggtggatatttgcgaccgtattgtctttaaatcgaaaaagtacgagggtcctttcactactactagcgaagtcgtggacatgctctctacgtacctgccgaaacagcctgagggcgttatcctgttctatagcaaaggtccgaaatccaacatcgattttaagattaaaaaggaaaacaccattgatcagacggctaatgtagttttccggtacatgtctagcgagccgatcatctttggcgaatcttctatctttgtagaatataaaaagttcagcaacgacaaaggattcccaaaagaatacgggtccgggaaaatcgtcttatacaacggtgttaactacttgaacaacatctattgcctggaatatatcaatactcacaatgaagttggtattaaatcagtggttgttccgataaaattcatcgcggaatttctggtcaatggcgaaatcctgaaaccccgcattgataagaccatgaaatacataaactccgaagactactacggtaaccagcataacatcatcgtggaacacctgagagatcagagtatcaaaatcggcgacattttcaatgaggacaagttaagcgacgtgggccatcaatacgcaaacaacgacaaattccgtctgaacccggaggtttcctatttcaccaacaaacgtacccgaggtccgcttggcatcctctccaattacgtaaaaaccctgctgatttctatgtattgttcaaaaacgttcctggatgacagcaacaaaaggaaggtactggctatcgatttcggtaacggcgcggatctggaaaagtacttttacggtgaaatcgctctgttagtcgcaactgatccggacgccgacgcaattgctcgcggaaatgaacgttacaacaaactgaactccggtattaaaacaaagtattataaattcgactatatccaggagactatccgctctgatactttcgtgagcagcgtgcgtgaggttttttactttggtaaattcaacattattgactggcagtttgcgatccactacagctttcacccgcgtcactatgcgaccgttatgaataacctatcggaactcacggctagcggcggcaaagtgctgattactactatggacggtgacaaactgtctaagctgaccgataagaaaaccttcatcatccacaaaaacttgccaagttctgagaactatatgtctgttgaaaaaattgcggacgaccgcatcgtcgtttacaacccatctaccatgtccacccctatgacagagtacatcatcaaaaagaacgacatagttcgtgttttcaacgaatacggcttcgtactggtagataacgtcgattttgctaccattatcgagcgttcgaaaaaattcattaacggtgcttccactatggaagatcgtccgtccactcgtaacttttttgaattaaaccgtggcgcaatcaaatgcgaagggctggatgtggaagacctcctgtcttactacgttgtatacgtcttctctaaacgctaaccggcttatcggtcagtttcacctgatttacgtaaaaacccgcttcggcgggtttttgcttttggaggggcagaaagatgaatgactgtccacgacgctatacccaaaagaaatgttgacaattaatcatcggctcgtataatgtgtggaattgtgagcgctcacaattgtcaataaaggcatataaaaggaggttaataacatgaaagttaaagtaaaacatcttaatcatgctaaggaggttttctaatggatgagatcgttaagaacattcgtgaaggtacgcatgtgcttttgccattttacgaaactctcccggaactgaatctgtccttaggcaaaagccctctaccctctctggagtatggggccaactacttcctgcaaatctcacgcgtcaacgacctgaatcgaatgccgaccgacatgctgaaactgttcactcacgatataatgctgccggaaagtgatctggacaaagtatatgaaatcctgaaaatcaacagcgttaagtactacggacggtcgaccaaagcggacgctgttgtagcagatctgtctgctcgcaacaaactctttaaacgtgaacgtgacgctattaagtccaacaaccacctgacagagaacaatctctatatctctgactacaaaatgttgactttcgatgtgttccgtccgctgtttgatttcgtgaacgaaaaatattgcattatcaaactgccgaccctgttcggccgtggtgttattgacaccatgcgcatctactgtagcctcttcaagaatgtcagactactgaaatgcgtgtccgatagctggctgaaagacagcgcaatcatggtagcctcagacgtttgcaaaaagaacctggatctgtttatgtcccatgttaaatccgttactaagtctagctcgtggaaagatgttaacagcgtacagttttctattttgaacaaccctgttgacacggaatttatcaacaaattcctggagttctctaaccgtgtatacgaagcgctgtattacgtgcactccttactgtactcttctatgaccagcgatagtaagtctatcgaaaataaacaccagcgccgtctggtaaaactgctccttgggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttataataaccccttggggcctctaaacgggtcttgaggggttttttgc

SEQ ID NO: 26 - DNA, 4163 bp, Artificial Sequence
  Synthetic
aattgtgagcggataacaattacgagcttcatgcacagtgaaatcatgaaaaatttatttgctttgtgagcggataacaattataatatgtggaattgtgagcgctcacaattccacagcgaaaaatcaataaggaggcaacaagatgtgcgaaaaacatcttaatcatgcggtggagggtttctaatgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaaccggcttatcggtcagtttcacctgatttacgtaaaaacccgcttcggcgggtttttgcttttggaggggcagaaagatgaatgactgtccacgacgctatacccaaaagaaatgttgacaattaatcatcggctcgtataatgtgtggaattgtgagcgctcacaattgcgaaaaatcaataaggaggcaacaagatgtgcgaaaaacatcttaatcatgcgccggaggttttctaatggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttataataaccccttggggcctctaaacgggtcttgaggggttttttgc

SEQ ID NO: 27 - DNA, 4175 bp, Artificial Sequence
  Synthetic
aattgtgagcggataacaattacgagcttcatgcacagtgaaatcatgaaaaatttatttgctttgtgagcggataacaattataatatgtggaattgtgagcgctcacaattccacagcgaaaaatcaataaggaggcaacaagatgtgcgaaaaacatcttaatcatgcggaggatcgtttctaatgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaaccggcttatcggtcagtttcacctgatttacgtaaaaacccgcttcggcgggtttttgcttttggaggggcagaaagatgaatgactgtccacgacgctatacccaaaagaaatgttgacaattaatcatcggctcgtataatgtgtggaattgtgagcgctcacaattgtcaataaaggcatataaaaggaggttaataacatgaaagttaaagtaaaacatcttaatcatgcgggggagtctttctaatggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttataataaccccttggggcctctaaacgggtcttgaggggttttttgc

SEQ ID NO: 28 - DNA, 4175 bp, Artificial Sequence
  Synthetic
aattgtgagcggataacaattacgagcttcatgcacagtgaaatcatgaaaaatttatttgctttgtgagcggataacaattataatatgtggaattgtgagcgctcacaattccacagcgaaaaatcaataaggaggcaacaagatgtgcgaaaaacatcttaatcatgcggaggatcgtttctaatgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaaccggcttatcggtcagtttcacctgatttacgtaaaaacccgcttcggcgggtttttgcttttggaggggcagaaagatgaatgactgtccacgacgctatacccaaaagaaatgttgacaattaatcatcggctcgtataatgtgtggaattgtgagcgctcacaattgtcaataaaggcatataaaaggaggttaataacatgaaagttaaagtaaaacatcttaatcatgcgggggagtgtttctaatggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttataataaccccttggggcctctaaacgggtcttgaggggttttttgc

SEQ ID NO: 29 - protein, 844 aa, Unknown
  Vaccinia virus
MDANVVSSSTIATYIDALAKNASELEQRSTAYEINNELELVFIKPPLITLTNVVNISTIQESFIRFTVTNKEGVKIRTKIPLSKVHGLDVKNVQLVDAIDNIVWEKKSLVTENRLHKECLLRLSTEERHIFLDYKKYGSSIRLELVNLIQAKTKNFTIDFKLKYFLGSGAQSKSSLLHAINHPKSRPNTSLEIEFTPRDNETVPYDELIKELTTLSRHIFMASPENVILSPPINAPIKTFMLPKQDIVGLDLENLYAVTKTDGIPITIRVTSNGLYCYFTHLGYIIRYPVKRIIDSEVVVFGEAVKDKNWTVYLIKLIEPVNAINDRLEESKYVESKLVDICDRIVFKSKKYEGPFTTTSEVVDMLSTYLPKQPEGVILFYSKGPKSNIDFKIKKENTIDQTANVVFRYMSSEPIIFGESSIFVEYKKFSNDKGFPKEYGSGKIVLYNGVNYLNNIYCLEYINTHNEVGIKSVVVPIKFIAEFLVNGEILKPRIDKTMKYINSEDYYGNQHNIIVEHLRDQSIKIGDIFNEDKLSDVGHQYANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTRNFFELNRGAIKCEGLDVEDLLSYYVVYVFSKR

SEQ ID NO: 30 - DNA, 2535 bp, Unknown
  Vaccinia virus
atggatgccaacgtagtatcatcttctactattgcgacgtatatagacgctttagcgaagaatgcttcggaattagaacagaggtctaccgcatacgaaataaataatgaattggaactagtatttattaagccgccattgattactttgacaaatgtagtgaatatctctacgattcaggaatcgtttattcgatttaccgttactaataaggaaggtgttaaaattagaactaagattccattatctaaggtacatggtctagatgtaaaaaatgtacagttagtagatgctatagataacatagtttgggaaaagaaatcattagtgacggaaaatcgtcttcacaaagaatgcttgttgagactatcgacagaggaacgtcatatatttttggattacaagaaatatggatcctctatccgactagaattagtcaatcttattcaagcaaaaacaaaaaactttacgatagactttaagctaaaatattttctaggatccggtgcccagtctaaaagttctttattacacgctattaatcatccaaagtcaaggcctaatacatctctggaaatagaatttacacctagagacaatgaaacagttccatatgatgaactaataaaggaattgacgactctctcgcgtcatatatttatggcttctccagagaatgtaattctttctccgcctattaacgcgcctataaaaacctttatgttgcctaaacaagatatagtaggtttggatctggaaaatctatatgccgtaactaagactgacggcattcctataactatcagagttacatcaaacgggttgtattgttattttacacatcttggttatattattagatatcctgttaagagaataatagattccgaagtagtagtctttggtgaggcagttaaggataagaactggaccgtatatctcattaagctaatagagcctgtgaatgcaatcaatgatagactagaagaaagtaagtatgttgaatctaaactagtggatatttgtgatcggatagtattcaagtcaaagaaatacgaaggtccgtttactacaactagtgaagtcgtcgatatgttatctacatatttaccaaagcaaccagaaggtgttattctgttctattcaaagggacctaaatctaacattgattttaaaattaaaaaggaaaatactatagaccaaactgcaaatgtagtatttaggtacatgtccagtgaaccaattatctttggagagtcgtctatctttgtagagtataagaaatttagcaacgataaaggctttcctaaagaatatggttctggtaagattgtgttatataacggcgttaattatctaaataatatctattgtttggaatatattaatacacataatgaagtgggtattaagtccgtggttgtacctattaagtttatagcagaattcttagttaatggagaaatacttaaacctagaattgataaaaccatgaaatatattaactcagaagattattatggaaatcaacataatatcatagtcgaacatttaagagatcaaagcatcaaaataggagatatctttaacgaggataaactatcggatgtgggacatcaatacgccaataatgataaatttagattaaatccagaagttagttattttacgaataaacgaactagaggaccgttgggaattttatcaaactacgtcaagactcttcttatttctatgtattgttccaaaacatttttagacgattccaacaaacgaaaggtattggcgattgattttggaaacggtgctgacctggaaaaatacttttatggagagattgcgttattggtagcgacggatccggatgctgatgctatagctagaggaaatgaaagatacaacaaattaaactctggaattaaaaccaagtactacaaatttgactacattcaggaaactattcgatccgatacatttgtctctagtgtcagagaagtattctattttggaaagtttaatatcatcgactggcagtttgctatccattattcttttcatccgagacattatgctaccgtcatgaataacttatccgaactaactgcttctggaggcaaggtattaatcactaccatggacggagacaaattatcaaaattaacagataaaaagacttttataattcataagaatttacctagtagcgaaaactatatgtctgtagaaaaaatagctgatgatagaatagtggtatataatccatcaacaatgtctactccaatgactgaatacattatcaaaaagaacgatatagtcagagtgtttaacgaatacggatttgttcttgtagataacgttgatttcgctacaattatagaacgaagtaaaaagtttattaatggcgcatctacaatggaagatagaccatctacaagaaactttttcgaactaaatagaggagccattaaatgtgaaggtttagatgtcgaagacttacttagttactatgttgtttatgtcttttctaagcggtaa

SEQ ID NO: 31 - protein, 287 aa, Unknown
  Vaccinia virus
MDEIVKNIREGTHVLLPFYETLPELNLSLGKSPLPSLEYGANYFLQISRVNDLNRMPTDMLKLFTHDIMLPESDLDKVYEILKINSVKYYGRSTKADAVVADLSARNKLFKRERDAIKSNNHLTENNLYISDYKMLTFDVFRPLFDFVNEKYCIIKLPTLFGRGVIDTMRIYCSLFKNVRLLKCVSDSWLKDSAIMVASDVCKKNLDLFMSHVKSVTKSSSWKDVNSVQFSILNNPVDTEFINKFLEFSNRVYEALYYVHSLLYSSMTSDSKSIENKHQRRLVKLLL

SEQ ID NO: 32 - DNA, 864 bp, Unknown
  Vaccinia virus
atggatgaaattgtaaaaaatatccgggagggaacgcatgtccttcttccattttatgaaacattgccagaacttaatctgtctctaggtaaaagcccattacctagtctggaatacggagctaattactttcttcagatttctagagttaatgatctaaatagaatgccgaccgacatgttaaaactttttacacatgatatcatgttaccagaaagcgatctagataaagtctatgaaattttaaagattaatagcgtaaagtattatgggaggagtactaaagcggacgccgtagttgccgacctcagcgcacgcaataaactgttcaaacgtgaacgagatgctattaaatctaataatcatctcactgaaaacaatctatacattagcgattataagatgttaaccttcgacgtgtttcgaccattatttgattttgtaaacgaaaaatattgtattattaaacttccaactttattcggtagaggtgtaatcgatactatgagaatatattgtagtctctttaaaaatgttagactgctaaaatgcgtaagcgatagctggttaaaagatagcgccattatggtggctagtgatgtttgtaaaaaaaatttggatttatttatgtctcatgttaagtccgtcactaagtcttcttcttggaaggatgtgaacagtgttcaatttagtattttaaacaatccagtggatacggaattcattaataagttcttagagttttcgaatagagtatacgaagctctctattacgttcactcgttgctttattctagtatgacttctgattcaaaaagtatcgaaaacaaacatcagagaagactagttaaactactgctgtga

SEQ ID NO: 33 - DNA, 2529 bp, Artificial Sequence
  Synthetic
gacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgc

SEQ ID NO: 34 - DNA, 2529 bp, Artificial Sequence
  Synthetic
gacgccaacgtagtgagctcgtccacgattgctacatacatcgacgcactggctaaaaacgcgagtgaattagagcaacgttcaaccgcctatgaaatcaacaacgaacttgagctcgtctttattaagcctccgctaatcaccctgactaacgttgttaatatatctaccatccaggaaagcttcattcgcttcactgttactaacaaagaaggcgtaaaaatcaggactaaaatcccattgtctaaggtgcacgggctggatgtgaaaaacgttcagctggttgacgctattgacaacatcgtatgggaaaagaaatccctcgtaaccgaaaaccgtctgcataaagaatgtctgctgcgtctgagcacggaggaacgacacatctttctggattacaaaaaatatggtagttctattcgtctggagctggtgaacctgatccaggcaaagaccaaaaatttcacaattgacttcaaactaaaatactttctgggctccggtgcgcagagcaaatcttccctgttgcatgctatcaaccacccgaaaagccgcccgaatacttctctggaaatcgagttcaccccccgcgataacgaaactgtcccatacgatgagcttattaaggaactgaccacgctgtcccgtcacatttttatggcgagcccggaaaacgttatattatcgccgcctatcaacgctccgatcaagaccttcatgttgccgaaacaagacatcgtcggtctggatctggagaacctgtacgcagttactaaaaccgacggcatccccatcactatcagagtaacgtcaaacggattgtattgctatttcacccatctgggttacattattcgttacccggtgaaacgcatcatagattctgaagttgttgttttcggcgaagccgtaaaggacaaaaactggaccgtctatctgatcaagctaatcgaaccggttaatgctatcaacgatcggctggaagaatcgaaatacgtagaatctaaactggtggatatttgcgaccgtattgtctttaaatcgaaaaagtacgagggtcctttcactactactagcgaagtcgtggacatgctctctacgtacctgccgaaacagcctgagggcgttatcctgttctatagcaaaggtccgaaatccaacatcgattttaagattaaaaaggaaaacaccattgatcagacggctaatgtagttttccggtacatgtctagcgagccgatcatctttggcgaatcttctatctttgtagaatataaaaagttcagcaacgacaaaggattcccaaaagaatacgggtccgggaaaatcgtcttatacaacggtgttaactacttgaacaacatctattgcctggaatatatcaatactcacaatgaagttggtattaaatcagtggttgttccgataaaattcatcgcggaatttctggtcaatggcgaaatcctgaaaccccgcattgataagaccatgaaatacataaactccgaagactactacggtaaccagcataacatcatcgtggaacacctgagagatcagagtatcaaaatcggcgacattttcaatgaggacaagttaagcgacgtgggccatcaatacgcaaacaacgacaaattccgtctgaacccggaggtttcctatttcaccaacaaacgtacccgaggtccgcttggcatcctctccaattacgtaaaaaccctgctgatttctatgtattgttcaaaaacgttcctggatgacagcaacaaaaggaaggtactggctatcgatttcggtaacggcgcggatctggaaaagtacttttacggtgaaatcgctctgttagtcgcaactgatccggacgccgacgcaattgctcgcggaaatgaacgttacaacaaactgaactccggtattaaaacaaagtattataaattcgactatatccaggagactatccgctctgatactttcgtgagcagcgtgcgtgaggttttttactttggtaaattcaacattattgactggcagtttgcgatccactacagctttcacccgcgtcactatgcgaccgttatgaataacctatcggaactcacggctagcggcggcaaagtgctgattactactatggacggtgacaaactgtctaagctgaccgataagaaaaccttcatcatccacaaaaacttgccaagttctgagaactatatgtctgttgaaaaaattgcggacgaccgcatcgtcgtttacaacccatctaccatgtccacccctatgacagagtacatcatcaaaaagaacgacatagttcgtgttttcaacgaatacggcttcgtactggtagataacgtcgattttgctaccattatcgagcgttcgaaaaaattcattaacggtgcttccactatggaagatcgtccgtccactcgtaacttttttgaattaaaccgtggcgcaatcaaatgcgaagggctggatgtggaagacctcctgtcttactacgttgtatacgtcttctctaaacgc

SEQ ID NO: 35 - DNA, 858 bp, Artificial Sequence
  Synthetic
gatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctg

SEQ ID NO: 36 - DNA, 858 bp, Artificial Sequence
  Synthetic
gatgagatcgttaagaacattcgtgaaggtacgcatgtgcttttgccattttacgaaactctcccggaactgaatctgtccttaggcaaaagccctctaccctctctggagtatggggccaactacttcctgcaaatctcacgcgtcaacgacctgaatcgaatgccgaccgacatgctgaaactgttcactcacgatataatgctgccggaaagtgatctggacaaagtatatgaaatcctgaaaatcaacagcgttaagtactacggacggtcgaccaaagcggacgctgttgtagcagatctgtctgctcgcaacaaactctttaaacgtgaacgtgacgctattaagtccaacaaccacctgacagagaacaatctctatatctctgactacaaaatgttgactttcgatgtgttccgtccgctgtttgatttcgtgaacgaaaaatattgcattatcaaactgccgaccctgttcggccgtggtgttattgacaccatgcgcatctactgtagcctcttcaagaatgtcagactactgaaatgcgtgtccgatagctggctgaaagacagcgcaatcatggtagcctcagacgtttgcaaaaagaacctggatctgtttatgtcccatgttaaatccgttactaagtctagctcgtggaaagatgttaacagcgtacagttttctattttgaacaaccctgttgacacggaatttatcaacaaattcctggagttctctaaccgtgtatacgaagcgctgtattacgtgcactccttactgtactcttctatgaccagcgatagtaagtctatcgaaaataaacaccagcgccgtctggtaaaactgctcctt

SEQ ID NO: 37 - DNA, 68 bp, Artificial Sequence
  Synthetic
gcgaaaaatcaataaggaggcaacaagatgtgcgaaaaacatcttaatcatgcaggggagggtttcta

SEQ ID NO: 38 - DNA, 68 bp, Artificial Sequence
  Synthetic
gcgaaaaatcaataaggaggcaacaagatgtgcgaaaaacatcttaatcatgcatcggaccgtttcta

SEQ ID NO: 39 - protein, 383 aa, Escherichia coli
MFEPMELTNDAVIKVIGVGGGGGNAVEHMVRERIEGVEFFAVNTDAQALRKTAVGQTIQIGSGITKGLGAGANPEVGRNAADEDRDALRAALEGADMVFIAAGMGGGTGTGAAPVVAEVAKDLGILTVAVVTKPFNFEGKKRMAFAEQGITELSKHVDSLITIPNDKLLKVLGRGISLLDAFGAANDVLKGAVQGIAELITRPGLMNVDFADVRTVMSEMGYAMMGSGVASGEDRAEEAAEMAISSPLLEDIDLSGARGVLVNITAGFDLRLDEFETVGNTIRAFASDNATVVIGTSLDPDMNDELRVTVVATGIGMDKRPEITLVTNKQVQQPVMDRYQQHGMAPLTQEQKPVAKVVNDNAPQTAKEPDYLDIPAFLRKQAD

SEQ ID NO: 40 - protein, 384 aa, Escherichia coli
MAKHLFTSESVSEGHPDKIADQISDAVLDAILEQDPKARVACETYVKTGMVLVGGEITTSAWVDIEEITRNTVREIGYVHSDMGFDANSCAVLSAIGKQSPDINQGVDRADPLEQGAGDQGLMFGYATNETDVLMPAPITYAHRLVQRQAEVRKNGTLPWLRPDAKSQVTFQYDDGKIVGIDAVVLSTQHSEEIDQKSLQEAVMEEIIKPILPAEWLTSATKFFINPTGRFVIGGPMGDCGLTGRKIIVDTYGGMARHGGGAFSGKDPSKVDRSAAYAARYVAKNIVAAGLADRCEIQVSYAIGVAEPTSIMVETFGTEKVPSEQLTLLVREFFDLRPYGLIQMLDLLHPIYKETAAYGHFGREHFPWEKTDKAQLLRDAAGLK

SEQ ID NO: 41 - protein, 347 aa, Escherichia coli
MLKKFRGMFSNDLSIDLGTANTLIYVKGQGIVLNEPSVVAIRQDRAGSPKSVAAVGHDAKQMLGRTPGNIAAIRPMKDGVIADFFVTEKMLQHFIKQVHSNSFMRPSPRVLVCVPVGATQVERRAIRESAQGAGAREVFLIEEPMAAAIGAGLPVSEATGSMVVDIGGGTTEVAVISLNGVVYSSSVRIGGDRFDEAIINYVRRNYGSLIGEATAERIKHEIGSAYPGDEVREIEVRGRNLAEGVPRGFTLNSNEILEALQEPLTGIVSAVMVALEQCPPELASDISERGMVLTGGGALLRNLDRLLMEETGIPVVVAEDPLTCVARGGGKALEMIDMHGGDLFSEE

SEQ ID NO: 42 - DNA, 1152 bp, Escherichia coli
atgtttgaaccaatggaacttaccaatgacgcggtgattaaagtcatcggcgtcggcggcggcggcggtaatgctgttgaacacatggtgcgcgagcgcattgaaggtgttgaattcttcgcggtaaataccgatgcacaagcgctgcgtaaaacagcggttggacagacgattcaaatcggtagcggtatcaccaaaggactgggcgctggcgctaatccagaagttggccgcaatgcggctgatgaggatcgcgatgcattgcgtgcggcgctggaaggtgcagacatggtctttattgctgcgggtatgggtggtggtaccggtacaggtgcagcaccagtcgtcgctgaagtggcaaaagatttgggtatcctgaccgttgctgtcgtcactaagcctttcaactttgaaggcaagaagcgtatggcattcgcggagcaggggatcactgaactgtccaagcatgtggactctctgatcactatcccgaacgacaaactgctgaaagttctgggccgcggtatctccctgctggatgcgtttggcgcagcgaacgatgtactgaaaggcgctgtgcaaggtatcgctgaactgattactcgtccgggtttgatgaacgtggactttgcagacgtacgcaccgtaatgtctgagatgggctacgcaatgatgggttctggcgtggcgagcggtgaagaccgtgcggaagaagctgctgaaatggctatctcttctccgctgctggaagatatcgacctgtctggcgcgcgcggcgtgctggttaacatcacggcgggcttcgacctgcgtctggatgagttcgaaacggtaggtaacaccatccgtgcatttgcttccgacaacgcgactgtggttatcggtacttctcttgacccggatatgaatgacgagctgcgcgtaaccgttgttgcgacaggtatcggcatggacaaacgtcctgaaatcactctggtgaccaataagcaggttcagcagccagtgatggatcgctaccagcagcatgggatggctccgctgacccaggagcagaagccggttgctaaagtcgtgaatgacaatgcgccgcaaactgcgaaagagccggattatctggatatcccagcattcctgcgtaagcaagctgattaa

SEQ ID NO: 43 - DNA, 1155 bp, Escherichia coli
atggcaaaacacctttttacgtccgagtccgtctctgaagggcatcctgacaaaattgctgaccaaatttctgatgccgttttagacgcgatcctcgaacaggatccgaaagcacgcgttgcttgcgaaacctacgtaaaaaccggcatggttttagttggcggcgaaatcaccaccagcgcctgggtagacatcgaagagatcacccgtaacaccgttcgcgaaattggctatgtgcattccgacatgggctttgacgctaactcctgtgcggttctgagcgctatcggcaaacagtctcctgacatcaaccagggcgttgaccgtgccgatccgctggaacagggcgcgggtgaccagggtctgatgtttggctacgcaactaatgaaaccgacgtgctgatgccagcacctatcacctatgcacaccgtctggtacagcgtcaggctgaagtgcgtaaaaacggcactctgccgtggctgcgcccggacgcgaaaagccaggtgacttttcagtatgacgacggcaaaatcgttggtatcgatgctgtcgtgctttccactcagcactctgaagagatcgaccagaaatcgctgcaagaagcggtaatggaagagatcatcaagccaattctgcccgctgaatggctgacttctgccaccaaattcttcatcaacccgaccggtcgtttcgttatcggtggcccaatgggtgactgcggtctgactggtcgtaaaattatcgttgatacctacggcggcatggcgcgtcacggtggcggtgcattctctggtaaagatccatcaaaagtggaccgttccgcagcctacgcagcacgttatgtcgcgaaaaacatcgttgctgctggcctggccgatcgttgtgaaattcaggtttcctacgcaatcggcgtggctgaaccgacctccatcatggtagaaactttcggtactgagaaagtgccttctgaacaactgaccctgctggtacgtgagttcttcgacctgcgcccatacggtctgattcagatgctggatctgctgcacccgatctacaaagaaaccgcagcatacggtcactttggtcgtgaacatttcccgtgggaaaaaaccgacaaagcgcagctgctgcgcgatgctgccggtctgaagtaa

SEQ ID NO: 44 - DNA, 1044 bp, Escherichia coli
ttactcttcgctgaacaggtcgccgccgtgcatgtcgatcatttccagcgctttgccgccaccgcgcgccacacaggtcagcgggtcttcagcaacaacgactggaatgccggtttcttccattaacaaacggtcaaggttacgcagcagtgcgccaccaccggtgagcaccatgccgcgctcggagatgtcggaagccagttccggcgggcactgttccagtgcaaccattaccgcgctcacaataccggtcagcggttcctgcagtgcttcgaggatttcattggagttcagggtaaaaccgcgtggaacaccttctgccaggttacggccacgaacttcgatttcacggacttcatcgcccggataagccgaaccgatttcgtgcttgatacgttctgcggtggcttcaccgatcagagaaccgtaattacgacgcacatagttgatgatagcttcgtcgaaacggtcaccaccaatgcgcacagaagaggagtaaaccacaccgttcaaggagataacagcaacttcagtggtaccaccaccgatatcaaccaccatagaaccggtcgcttcagaaaccggcaggccagcaccaattgcggcagccatcggttcttcaatcaggaagacttcacgggcaccagcgccctgcgcggattcacgaattgcgcggcgttcaacctgggtcgcgccaaccggcacacaaaccagaacgcgcgggcttggacgcataaagctgttgctgtgcacttgtttgatgaagtgctggagcattttttcagtcacgaagaagtcggcgataacgccgtctttcattgggcgaatggcagcaatattgcccggcgtacggcccagcatctgcttcgcgtcatgacctactgcagctacgcttttcggtgaaccggcacgatcctgacgaatggccaccacggaaggctcattcaatacgatgccttgtccttttacataaatgagggtattcgcagtacccaggtcaatggacaagtcattggaaaacatgccacgaaattttttcaacat

SEQ ID NO: 45 - DNA, 71 bp, Artificial Sequence
  Synthetic
gcgaaaaatcaataaggaggcaacaagatgtgcgaaaaacatcttaatcatgcgagggatggtttctaatg

SEQ ID NO: 46 - DNA, 42 bp, Artificial Sequence
  Synthetic
ttgacatcgcatctttttgtaccatacttacagccattgtac

SEQ ID NO: 47 - DNA, 36 bp, Artificial Sequence
  Synthetic
tcgacatttatcccttgcggcgaatacttacagcca

SEQ ID NO: 48 - DNA, 36 bp, Artificial Sequence
  Synthetic
ttccctattaatcatccggctcgtataatgtgtgga

SEQ ID NO: 49 - DNA, 3883 bp, Artificial Sequence
  Synthetic
tcgacatttatcccttgcggcgaatacttacagccagcgaaaaatcaataaggaggcaacaagatgtgcgaaaaacatcttaatcatgcggtggagggtttctaatgaaacatcaccatcaccatcaccccatgagcgattacgacatccccactactgagaatctttattttcagggcgccgacgccaacgtagtgagctcgtccacgattgctacatacatcgacgcactggctaaaaacgcgagtgaattagagcaacgttcaaccgcctatgaaatcaacaacgaacttgagctcgtctttattaagcctccgctaatcaccctgactaacgttgttaatatatctaccatccaggaaagcttcattcgcttcactgttactaacaaagaaggcgtaaaaatcaggactaaaatcccattgtctaaggtgcacgggctggatgtgaaaaacgttcagctggttgacgctattgacaacatcgtatgggaaaagaaatccctcgtaaccgaaaaccgtctgcataaagaatgtctgctgcgtctgagcacggaggaacgacacatctttctggattacaaaaaatatggtagttctattcgtctggagctggtgaacctgatccaggcaaagaccaaaaatttcacaattgacttcaaactaaaatactttctgggctccggtgcgcagagcaaatcttccctgttgcatgctatcaaccacccgaaaagccgcccgaatacttctctggaaatcgagttcaccccccgcgataacgaaactgtcccatacgatgagcttattaaggaactgaccacgctgtcccgtcacatttttatggcgagcccggaaaacgttatattatcgccgcctatcaacgctccgatcaagaccttcatgttgccgaaacaagacatcgtcggtctggatctggagaacctgtacgcagttactaaaaccgacggcatccccatcactatcagagtaacgtcaaacggattgtattgctatttcacccatctgggttacattattcgttacccggtgaaacgcatcatagattctgaagttgttgttttcggcgaagccgtaaaggacaaaaactggaccgtctatctgatcaagctaatcgaaccggttaatgctatcaacgatcggctggaagaatcgaaatacgtagaatctaaactggtggatatttgcgaccgtattgtctttaaatcgaaaaagtacgagggtcctttcactactactagcgaagtcgtggacatgctctctacgtacctgccgaaacagcctgagggcgttatcctgttctatagcaaaggtccgaaatccaacatcgattttaagattaaaaaggaaaacaccattgatcagacggctaatgtagttttccggtacatgtctagcgagccgatcatctttggcgaatcttctatctttgtagaatataaaaagttcagcaacgacaaaggattcccaaaagaatacgggtccgggaaaatcgtcttatacaacggtgttaactacttgaacaacatctattgcctggaatatatcaatactcacaatgaagttggtattaaatcagtggttgttccgataaaattcatcgcggaatttctggtcaatggcgaaatcctgaaaccccgcattgataagaccatgaaatacataaactccgaagactactacggtaaccagcataacatcatcgtggaacacctgagagatcagagtatcaaaatcggcgacattttcaatgaggacaagttaagcgacgtgggccatcaatacgcaaacaacgacaaattccgtctgaacccggaggtttcctatttcaccaacaaacgtacccgaggtccgcttggcatcctctccaattacgtaaaaaccctgctgatttctatgtattgttcaaaaacgttcctggatgacagcaacaaaaggaaggtactggctatcgatttcggtaacggcgcggatctggaaaagtacttttacggtgaaatcgctctgttagtcgcaactgatccggacgccgacgcaattgctcgcggaaatgaacgttacaacaaactgaactccggtattaaaacaaagtattataaattcgactatatccaggagactatccgctctgatactttcgtgagcagcgtgcgtgaggttttttactttggtaaattcaacattattgactggcagtttgcgatccactacagctttcacccgcgtcactatgcgaccgttatgaataacctatcggaactcacggctagcggcggcaaagtgctgattactactatggacggtgacaaactgtctaagctgaccgataagaaaaccttcatcatccacaaaaacttgccaagttctgagaactatatgtctgttgaaaaaattgcggacgaccgcatcgtcgtttacaacccatctaccatgtccacccctatgacagagtacatcatcaaaaagaacgacatagttcgtgttttcaacgaatacggcttcgtactggtagataacgtcgattttgctaccattatcgagcgttcgaaaaaattcattaacggtgcttccactatggaagatcgtccgtccactcgtaacttttttgaattaaaccgtggcgcaatcaaatgcgaagggctggatgtggaagacctcctgtcttactacgttgtatacgtcttctctaaacgctaaaataattttgtttaactttaagaaggaggtatatccatggctagcatgactaaacatcttaatcatgcgggggagtctttctaatggatgagatcgttaagaacattcgtgaaggtacgcatgtgcttttgccattttacgaaactctcccggaactgaatctgtccttaggcaaaagccctctaccctctctggagtatggggccaactacttcctgcaaatctcacgcgtcaacgacctgaatcgaatgccgaccgacatgctgaaactgttcactcacgatataatgctgccggaaagtgatctggacaaagtatatgaaatcctgaaaatcaacagcgttaagtactacggacggtcgaccaaagcggacgctgttgtagcagatctgtctgctcgcaacaaactctttaaacgtgaacgtgacgctattaagtccaacaaccacctgacagagaacaatctctatatctctgactacaaaatgttgactttcgatgtgttccgtccgctgtttgatttcgtgaacgaaaaatattgcattatcaaactgccgaccctgttcggccgtggtgttattgacaccatgcgcatctactgtagcctcttcaagaatgtcagactactgaaatgcgtgtccgatagctggctgaaagacagcgcaatcatggtagcctcagacgtttgcaaaaagaacctggatctgtttatgtcccatgttaaatccgttactaagtctagctcgtggaaagatgttaacagcgtacagttttctattttgaacaaccctgttgacacggaatttatcaacaaattcctggagttctctaaccgtgtatacgaagcgctgtattacgtgcactccttactgtactcttctatgaccagcgatagtaagtctatcgaaaataaacaccagcgccgtctggtaaaactgctccttgggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 50 - DNA, 3874 bp, Artificial Sequence
  Synthetic
ttgacatcgcatctttttgtaccatacttacagccattgtacgcgaaaaatcaataaggaggcaacaagatgtgcgaaaaacatcttaatcatgcggtggagggtttctaatgaaacatcaccatcaccatcaccccatgagcgattacgacatccccactactgagaatctttattttcagggcgccgacgccaacgtagtgagctcgtccacgattgctacatacatcgacgcactggctaaaaacgcgagtgaattagagcaacgttcaaccgcctatgaaatcaacaacgaacttgagctcgtctttattaagcctccgctaatcaccctgactaacgttgttaatatatctaccatccaggaaagcttcattcgcttcactgttactaacaaagaaggcgtaaaaatcaggactaaaatcccattgtctaaggtgcacgggctggatgtgaaaaacgttcagctggttgacgctattgacaacatcgtatgggaaaagaaatccctcgtaaccgaaaaccgtctgcataaagaatgtctgctgcgtctgagcacggaggaacgacacatctttctggattacaaaaaatatggtagttctattcgtctggagctggtgaacctgatccaggcaaagaccaaaaatttcacaattgacttcaaactaaaatactttctgggctccggtgcgcagagcaaatcttccctgttgcatgctatcaaccacccgaaaagccgcccgaatacttctctggaaatcgagttcaccccccgcgataacgaaactgtcccatacgatgagcttattaaggaactgaccacgctgtcccgtcacatttttatggcgagcccggaaaacgttatattatcgccgcctatcaacgctccgatcaagaccttcatgttgccgaaacaagacatcgtcggtctggatctggagaacctgtacgcagttactaaaaccgacggcatccccatcactatcagagtaacgtcaaacggattgtattgctatttcacccatctgggttacattattcgttacccggtgaaacgcatcatagattctgaagttgttgttttcggcgaagccgtaaaggacaaaaactggaccgtctatctgatcaagctaatcgaaccggttaatgctatcaacgatcggctggaagaatcgaaatacgtagaatctaaactggtggatatttgcgaccgtattgtctttaaatcgaaaaagtacgagggtcctttcactactactagcgaagtcgtggacatgctctctacgtacctgccgaaacagcctgagggcgttatcctgttctatagcaaaggtccgaaatccaacatcgattttaagattaaaaaggaaaacaccattgatcagacggctaatgtagttttccggtacatgtctagcgagccgatcatctttggcgaatcttctatctttgtagaatataaaaagttcagcaacgacaaaggattcccaaaagaatacgggtccgggaaaatcgtcttatacaacggtgttaactacttgaacaacatctattgcctggaatatatcaatactcacaatgaagttggtattaaatcagtggttgttccgataaaattcatcgcggaatttctggtcaatggcgaaatcctgaaaccccgcattgataagaccatgaaatacataaactccgaagactactacggtaaccagcataacatcatcgtggaacacctgagagatcagagtatcaaaatcggcgacattttcaatgaggacaagttaagcgacgtgggccatcaatacgcaaacaacgacaaattccgtctgaacccggaggtttcctatttcaccaacaaacgtacccgaggtccgcttggcatcctctccaattacgtaaaaaccctgctgatttctatgtattgttcaaaaacgttcctggatgacagcaacaaaaggaaggtactggctatcgatttcggtaacggcgcggatctggaaaagtacttttacggtgaaatcgctctgttagtcgcaactgatccggacgccgacgcaattgctcgcggaaatgaacgttacaacaaactgaactccggtattaaaacaaagtattataaattcgactatatccaggagactatccgctctgatactttcgtgagcagcgtgcgtgaggttttttactttggtaaattcaacattattgactggcagtttgcgatccactacagctttcacccgcgtcactatgcgaccgttatgaataacctatcggaactcacggctagcggcggcaaagtgctgattactactatggacggtgacaaactgtctaagctgaccgataagaaaaccttcatcatccacaaaaacttgccaagttctgagaactatatgtctgttgaaaaaattgcggacgaccgcatcgtcgtttacaacccatctaccatgtccacccctatgacagagtacatcatcaaaaagaacgacatagttcgtgttttcaacgaatacggcttcgtactggtagataacgtcgattttgctaccattatcgagcgttcgaaaaaattcattaacggtgcttccactatggaagatcgtccgtccactcgtaacttttttgaattaaaccgtggcgcaatcaaatgcgaagggctggatgtggaagacctcctgtcttactacgttgtatacgtcttctctaaacgctaagcgaaaaatcaataaggaggcaacaagatgtgcgaaaaacatcttaatcatgcgagggatggtttctaatggatgagatcgttaagaacattcgtgaaggtacgcatgtgcttttgccattttacgaaactctcccggaactgaatctgtccttaggcaaaagccctctaccctctctggagtatggggccaactacttcctgcaaatctcacgcgtcaacgacctgaatcgaatgccgaccgacatgctgaaactgttcactcacgatataatgctgccggaaagtgatctggacaaagtatatgaaatcctgaaaatcaacagcgttaagtactacggacggtcgaccaaagcggacgctgttgtagcagatctgtctgctcgcaacaaactctttaaacgtgaacgtgacgctattaagtccaacaaccacctgacagagaacaatctctatatctctgactacaaaatgttgactttcgatgtgttccgtccgctgtttgatttcgtgaacgaaaaatattgcattatcaaactgccgaccctgttcggccgtggtgttattgacaccatgcgcatctactgtagcctcttcaagaatgtcagactactgaaatgcgtgtccgatagctggctgaaagacagcgcaatcatggtagcctcagacgtttgcaaaaagaacctggatctgtttatgtcccatgttaaatccgttactaagtctagctcgtggaaagatgttaacagcgtacagttttctattttgaacaaccctgttgacacggaatttatcaacaaattcctggagttctctaaccgtgtatacgaagcgctgtattacgtgcactccttactgtactcttctatgaccagcgatagtaagtctatcgaaaataaacaccagcgccgtctggtaaaactgctccttgggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 51 - DNA, 3868 bp, Artificial Sequence
  Synthetic
tcgacatttatcccttgcggcgaatacttacagccagcgaaaaatcaataaggaggcaacaagatgtgcgaaaaacatcttaatcatgcggtggagggtttctaatgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaagcgaaaaatcaataaggaggcaacaagatgtgcgaaaaacatcttaatcatgcgagggatggtttctaatggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 52 - DNA, 3883 bp, Artificial Sequence
  Synthetic
tcgacatttatcccttgcggcgaatacttacagccagcgaaaaatcaataaggaggcaacaagatgtgcgaaaaacatcttaatcatgcggtggagggtttctaatgaaacatcaccatcaccatcaccccatgagcgattacgacatccccactactgagaatctttattttcagggcgccgacgctaatgtcgtgtcttcttctaccatcgcaacctatattgacgctctggcaaaaaacgcctcggaactggaacaacgctcaaccgcgtatgaaatcaacaatgaactggaactggtgtttatcaaaccgccgctgattacgctgaccaacgtggttaatatcagcaccattcaggaatcttttattcgtttcacggttaccaacaaagaaggcgtcaaaatccgcacgaaaattccgctgagcaaagttcatggtctggatgtgaaaaacgttcaactggtcgacgcaatcgataatattgtgtgggaaaagaaaagcctggttaccgaaaatcgtctgcataaagaatgcctgctgcgtctgagcacggaagaacgccacatctttctggactataaaaaatacggcagctctatccgcctggaactggtgaacctgatccaggctaaaaccaaaaacttcacgatcgatttcaaactgaaatattttctgggcagtggtgctcaatccaaaagttccctgctgcatgcgatcaaccacccgaaaagtcgtccgaatacctccctggaaattgaattcaccccgcgcgacaacgaaacggtgccgtacgatgaactgattaaagaactgaccacgctgtcacgtcatatctttatggcgtcgccggaaaacgttattctgagcccgccgatcaatgccccgattaaaaccttcatgctgccgaaacaggacattgttggcctggatctggaaaacctgtatgcggtcacgaaaaccgatggtattccgatcaccattcgcgtgacgtcgaatggcctgtattgctactttacccacctgggttatattatccgttacccggttaaacgcattatcgactccgaagtcgtggttttcggcgaagcggtcaaagataaaaattggaccgtgtatctgatcaaactgattgaaccggtgaacgccatcaacgatcgtctggaagaatcaaaatacgtggaatcgaaactggttgacatctgtgatcgcatcgttttcaaaagcaaaaaatacgaaggtccgttcaccacgacctctgaagtcgtggatatgctgagtacctatctgccgaaacagccggaaggcgtgatcctgttttacagcaaaggtccgaaatctaacatcgacttcaaaatcaaaaaagaaaacaccatcgatcaaacggccaatgttgtctttcgttatatgtcatcggaaccgattatctttggcgaaagctctatcttcgtggaatacaaaaaattctcgaacgataaaggcttcccgaaagaatacggcagcggtaaaattgtcctgtataacggtgtgaattacctgaacaatatctattgcctggaatacattaacacccataatgaagttggcattaaatctgtggttgtcccgatcaaatttattgcagaattcctggtcaacggtgaaatcctgaaaccgcgtattgacaaaaccatgaaatacatcaacagtgaagattactacggtaaccagcataacatcatcgtggaacacctgcgcgaccaatctatcaaaatcggcgatatcttcaacgaagacaaactgagtgatgtcggtcaccagtatgcgaacaatgataaatttcgtctgaacccggaagtgtcctacttcaccaataaacgtacgcgcggcccgctgggtatcctgtcaaattatgtcaaaaccctgctgatttcaatgtactgttcgaaaacgtttctggatgacagcaacaaacgcaaagttctggccattgactttggcaatggtgcagatctggaaaaatatttctacggcgaaatcgctctgctggttgcgaccgatccggacgcggatgccattgcacgtggcaacgaacgctataacaaactgaattctggtatcaaaaccaaatactacaaattcgactacatccaggaaaccattcgtagtgatacgttcgtgagttccgttcgcgaagtcttttatttcggcaaattcaacatcatcgattggcaattcgccatccattattctttccatccgcgtcactacgcaaccgtgatgaacaatctgagtgaactgacggcttccggcggtaaagttctgattacgacgatggatggtgataaactgtccaaactgaccgataagaaaaccttcattatccacaaaaacctgccgtcatcggaaaactacatgtcagtggaaaaaatcgccgatgaccgcattgtggtttataacccgagcacgatgtctaccccgatgacggaatacatcattaagaaaaacgatatcgtccgtgtgtttaatgaatacggtttcgttctggtcgacaacgttgattttgcaaccattatcgaacgcagcaaaaaattcatcaatggcgcttccacgatggaagatcgtccgtcaacgcgcaactttttcgaactgaatcgcggtgcaattaaatgtgaaggtctggatgtggaagatctgctgtcctattatgtcgtgtatgtgttctctaaacgctaaaataattttgtttaactttaagaaggaggtatatccatggctagcatgactaaacatcttaatcatgcgggggagtctttctaatggatgaaatcgtcaaaaatatccgcgaaggcacgcacgtcctgctgccgttctatgaaaccctgccggaactgaatctgtcactgggcaaatctccgctgccgagtctggaatatggtgcaaactactttctgcagatttctcgtgtgaacgatctgaatcgcatgccgaccgacatgctgaaactgttcacgcatgatatcatgctgccggaaagcgatctggacaaagtctacgaaatcctgaaaatcaactccgttaaatactacggccgttcaaccaaagcggatgccgtggttgcagacctgtccgctcgcaataaactgtttaaacgtgaacgcgatgctattaaatcgaacaatcacctgaccgaaaacaacctgtacatcagcgattacaaaatgctgacgtttgacgtgttccgtccgctgttcgatttcgttaacgaaaaatactgcatcatcaaactgccgaccctgtttggccgtggtgtgattgatacgatgcgcatctactgcagcctgttcaaaaatgtccgcctgctgaaatgtgtgtcggatagctggctgaaagactctgcgattatggtggccagtgacgtttgtaagaaaaacctggacctgtttatgtcccatgtcaaatcagtgaccaaaagctctagttggaaagacgttaattcggtccaatttagcattctgaacaatccggttgatacggaattcatcaacaaattcctggaattctctaaccgtgtttacgaagcactgtattacgtccacagtctgctgtactcctcaatgacctcggactccaaatccatcgaaaataaacatcaacgccgcctggtgaaactgctgctggggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 53 - DNA, 3883 bp, Artificial Sequence
  Synthetic
ttccctattaatcatccggctcgtataatgtgtggagcgaaaaatcaataaggaggcaacaagatgtgcgaaaaacatcttaatcatgcggtggagggtttctaatgaaacatcaccatcaccatcaccccatgagcgattacgacatccccactactgagaatctttattttcagggcgccgacgccaacgtagtgagctcgtccacgattgctacatacatcgacgcactggctaaaaacgcgagtgaattagagcaacgttcaaccgcctatgaaatcaacaacgaacttgagctcgtctttattaagcctccgctaatcaccctgactaacgttgttaatatatctaccatccaggaaagcttcattcgcttcactgttactaacaaagaaggcgtaaaaatcaggactaaaatcccattgtctaaggtgcacgggctggatgtgaaaaacgttcagctggttgacgctattgacaacatcgtatgggaaaagaaatccctcgtaaccgaaaaccgtctgcataaagaatgtctgctgcgtctgagcacggaggaacgacacatctttctggattacaaaaaatatggtagttctattcgtctggagctggtgaacctgatccaggcaaagaccaaaaatttcacaattgacttcaaactaaaatactttctgggctccggtgcgcagagcaaatcttccctgttgcatgctatcaaccacccgaaaagccgcccgaatacttctctggaaatcgagttcaccccccgcgataacgaaactgtcccatacgatgagcttattaaggaactgaccacgctgtcccgtcacatttttatggcgagcccggaaaacgttatattatcgccgcctatcaacgctccgatcaagaccttcatgttgccgaaacaagacatcgtcggtctggatctggagaacctgtacgcagttactaaaaccgacggcatccccatcactatcagagtaacgtcaaacggattgtattgctatttcacccatctgggttacattattcgttacccggtgaaacgcatcatagattctgaagttgttgttttcggcgaagccgtaaaggacaaaaactggaccgtctatctgatcaagctaatcgaaccggttaatgctatcaacgatcggctggaagaatcgaaatacgtagaatctaaactggtggatatttgcgaccgtattgtctttaaatcgaaaaagtacgagggtcctttcactactactagcgaagtcgtggacatgctctctacgtacctgccgaaacagcctgagggcgttatcctgttctatagcaaaggtccgaaatccaacatcgattttaagattaaaaaggaaaacaccattgatcagacggctaatgtagttttccggtacatgtctagcgagccgatcatctttggcgaatcttctatctttgtagaatataaaaagttcagcaacgacaaaggattcccaaaagaatacgggtccgggaaaatcgtcttatacaacggtgttaactacttgaacaacatctattgcctggaatatatcaatactcacaatgaagttggtattaaatcagtggttgttccgataaaattcatcgcggaatttctggtcaatggcgaaatcctgaaaccccgcattgataagaccatgaaatacataaactccgaagactactacggtaaccagcataacatcatcgtggaacacctgagagatcagagtatcaaaatcggcgacattttcaatgaggacaagttaagcgacgtgggccatcaatacgcaaacaacgacaaattccgtctgaacccggaggtttcctatttcaccaacaaacgtacccgaggtccgcttggcatcctctccaattacgtaaaaaccctgctgatttctatgtattgttcaaaaacgttcctggatgacagcaacaaaaggaaggtactggctatcgatttcggtaacggcgcggatctggaaaagtacttttacggtgaaatcgctctgttagtcgcaactgatccggacgccgacgcaattgctcgcggaaatgaacgttacaacaaactgaactccggtattaaaacaaagtattataaattcgactatatccaggagactatccgctctgatactttcgtgagcagcgtgcgtgaggttttttactttggtaaattcaacattattgactggcagtttgcgatccactacagctttcacccgcgtcactatgcgaccgttatgaataacctatcggaactcacggctagcggcggcaaagtgctgattactactatggacggtgacaaactgtctaagctgaccgataagaaaaccttcatcatccacaaaaacttgccaagttctgagaactatatgtctgttgaaaaaattgcggacgaccgcatcgtcgtttacaacccatctaccatgtccacccctatgacagagtacatcatcaaaaagaacgacatagttcgtgttttcaacgaatacggcttcgtactggtagataacgtcgattttgctaccattatcgagcgttcgaaaaaattcattaacggtgcttccactatggaagatcgtccgtccactcgtaacttttttgaattaaaccgtggcgcaatcaaatgcgaagggctggatgtggaagacctcctgtcttactacgttgtatacgtcttctctaaacgctaaaataattttgtttaactttaagaaggaggtatatccatggctagcatgactaaacatcttaatcatgcgggggagtctttctaatggatgagatcgttaagaacattcgtgaaggtacgcatgtgcttttgccattttacgaaactctcccggaactgaatctgtccttaggcaaaagccctctaccctctctggagtatggggccaactacttcctgcaaatctcacgcgtcaacgacctgaatcgaatgccgaccgacatgctgaaactgttcactcacgatataatgctgccggaaagtgatctggacaaagtatatgaaatcctgaaaatcaacagcgttaagtactacggacggtcgaccaaagcggacgctgttgtagcagatctgtctgctcgcaacaaactctttaaacgtgaacgtgacgctattaagtccaacaaccacctgacagagaacaatctctatatctctgactacaaaatgttgactttcgatgtgttccgtccgctgtttgatttcgtgaacgaaaaatattgcattatcaaactgccgaccctgttcggccgtggtgttattgacaccatgcgcatctactgtagcctcttcaagaatgtcagactactgaaatgcgtgtccgatagctggctgaaagacagcgcaatcatggtagcctcagacgtttgcaaaaagaacctggatctgtttatgtcccatgttaaatccgttactaagtctagctcgtggaaagatgttaacagcgtacagttttctattttgaacaaccctgttgacacggaatttatcaacaaattcctggagttctctaaccgtgtatacgaagcgctgtattacgtgcactccttactgtactcttctatgaccagcgatagtaagtctatcgaaaataaacaccagcgccgtctggtaaaactgctccttgggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 54 - DNA, 3868 bp, Artificial Sequence
  Synthetic
ttccctattaatcatccggctcgtataatgtgtggagcgaaaaatcaataaggaggcaacaagatgtgcgaaaaacatcttaatcatgcggtggagggtttctaatgaaacatcaccatcaccatcaccccatgagcgattacgacatccccactactgagaatctttattttcagggcgccgacgccaacgtagtgagctcgtccacgattgctacatacatcgacgcactggctaaaaacgcgagtgaattagagcaacgttcaaccgcctatgaaatcaacaacgaacttgagctcgtctttattaagcctccgctaatcaccctgactaacgttgttaatatatctaccatccaggaaagcttcattcgcttcactgttactaacaaagaaggcgtaaaaatcaggactaaaatcccattgtctaaggtgcacgggctggatgtgaaaaacgttcagctggttgacgctattgacaacatcgtatgggaaaagaaatccctcgtaaccgaaaaccgtctgcataaagaatgtctgctgcgtctgagcacggaggaacgacacatctttctggattacaaaaaatatggtagttctattcgtctggagctggtgaacctgatccaggcaaagaccaaaaatttcacaattgacttcaaactaaaatactttctgggctccggtgcgcagagcaaatcttccctgttgcatgctatcaaccacccgaaaagccgcccgaatacttctctggaaatcgagttcaccccccgcgataacgaaactgtcccatacgatgagcttattaaggaactgaccacgctgtcccgtcacatttttatggcgagcccggaaaacgttatattatcgccgcctatcaacgctccgatcaagaccttcatgttgccgaaacaagacatcgtcggtctggatctggagaacctgtacgcagttactaaaaccgacggcatccccatcactatcagagtaacgtcaaacggattgtattgctatttcacccatctgggttacattattcgttacccggtgaaacgcatcatagattctgaagttgttgttttcggcgaagccgtaaaggacaaaaactggaccgtctatctgatcaagctaatcgaaccggttaatgctatcaacgatcggctggaagaatcgaaatacgtagaatctaaactggtggatatttgcgaccgtattgtctttaaatcgaaaaagtacgagggtcctttcactactactagcgaagtcgtggacatgctctctacgtacctgccgaaacagcctgagggcgttatcctgttctatagcaaaggtccgaaatccaacatcgattttaagattaaaaaggaaaacaccattgatcagacggctaatgtagttttccggtacatgtctagcgagccgatcatctttggcgaatcttctatctttgtagaatataaaaagttcagcaacgacaaaggattcccaaaagaatacgggtccgggaaaatcgtcttatacaacggtgttaactacttgaacaacatctattgcctggaatatatcaatactcacaatgaagttggtattaaatcagtggttgttccgataaaattcatcgcggaatttctggtcaatggcgaaatcctgaaaccccgcattgataagaccatgaaatacataaactccgaagactactacggtaaccagcataacatcatcgtggaacacctgagagatcagagtatcaaaatcggcgacattttcaatgaggacaagttaagcgacgtgggccatcaatacgcaaacaacgacaaattccgtctgaacccggaggtttcctatttcaccaacaaacgtacccgaggtccgcttggcatcctctccaattacgtaaaaaccctgctgatttctatgtattgttcaaaaacgttcctggatgacagcaacaaaaggaaggtactggctatcgatttcggtaacggcgcggatctggaaaagtacttttacggtgaaatcgctctgttagtcgcaactgatccggacgccgacgcaattgctcgcggaaatgaacgttacaacaaactgaactccggtattaaaacaaagtattataaattcgactatatccaggagactatccgctctgatactttcgtgagcagcgtgcgtgaggttttttactttggtaaattcaacattattgactggcagtttgcgatccactacagctttcacccgcgtcactatgcgaccgttatgaataacctatcggaactcacggctagcggcggcaaagtgctgattactactatggacggtgacaaactgtctaagctgaccgataagaaaaccttcatcatccacaaaaacttgccaagttctgagaactatatgtctgttgaaaaaattgcggacgaccgcatcgtcgtttacaacccatctaccatgtccacccctatgacagagtacatcatcaaaaagaacgacatagttcgtgttttcaacgaatacggcttcgtactggtagataacgtcgattttgctaccattatcgagcgttcgaaaaaattcattaacggtgcttccactatggaagatcgtccgtccactcgtaacttttttgaattaaaccgtggcgcaatcaaatgcgaagggctggatgtggaagacctcctgtcttactacgttgtatacgtcttctctaaacgctaagcgaaaaatcaataaggaggcaacaagatgtgcgaaaaacatcttaatcatgcgagggatggtttctaatggatgagatcgttaagaacattcgtgaaggtacgcatgtgcttttgccattttacgaaactctcccggaactgaatctgtccttaggcaaaagccctctaccctctctggagtatggggccaactacttcctgcaaatctcacgcgtcaacgacctgaatcgaatgccgaccgacatgctgaaactgttcactcacgatataatgctgccggaaagtgatctggacaaagtatatgaaatcctgaaaatcaacagcgttaagtactacggacggtcgaccaaagcggacgctgttgtagcagatctgtctgctcgcaacaaactctttaaacgtgaacgtgacgctattaagtccaacaaccacctgacagagaacaatctctatatctctgactacaaaatgttgactttcgatgtgttccgtccgctgtttgatttcgtgaacgaaaaatattgcattatcaaactgccgaccctgttcggccgtggtgttattgacaccatgcgcatctactgtagcctcttcaagaatgtcagactactgaaatgcgtgtccgatagctggctgaaagacagcgcaatcatggtagcctcagacgtttgcaaaaagaacctggatctgtttatgtcccatgttaaatccgttactaagtctagctcgtggaaagatgttaacagcgtacagttttctattttgaacaaccctgttgacacggaatttatcaacaaattcctggagttctctaaccgtgtatacgaagcgctgtattacgtgcactccttactgtactcttctatgaccagcgatagtaagtctatcgaaaataaacaccagcgccgtctggtaaaactgctccttgggagcgcttggagccacccgcagttcgaaaaaggtggaggttctggcggtggatcgggaggttcagcgtggagccacccgcagttcgagaaataaccaggcatcaaataaaacgaaaggctcagtcgaaagactgggcctttcgttttatctgttgtttgtcggtgaacgctctctactagagtcacactggctcaccttcgggtgggcctttctgcgtttata

(19) United States
(12) Patent Application Publication
(10) Pub. No.: US 2024/0182877 A1
(43) Pub. Date: Jun. 6, 2024

(54) PRODUCTION OF VACCINIA CAPPING ENZYME

(71) Applicant: Ginkgo Bioworks, Inc., Boston, MA (US)

(72) Inventors: Josef Bober, South Boston, MA (US); Jeffrey Ian Boucher, Allston, MA (US); Justin Michael Gardin, Watertown, MA (US); Jason King, Newton, MA (US); Scott Marr, Boston, MA (US); Matthew McMahon, Melrose, MA (US); Krishnaben S. Patel, Melrose, MA (US); Abraham Waldman, Boston, MA (US)

(73) Assignee: Ginkgo Bioworks, Inc., Boston, MA (US)

(21) Appl. No.: 18/284,673

(22) PCT Filed: Mar. 29, 2022
(86) PCT No.: PCT/US2022/022303

§ 371 Date: Sep. 28, 2023

Related U.S. Application Data
(60) Provisional application No. 63/188,977, filed on May 14, 2021.
(60) Provisional application No. 63/167,249, filed on Mar. 29, 2021.

(51) Int. Cl. C12N 9/16; C12N 15/70

(52) U.S. Cl. CPC C12N 9/16; C12N 15/70; C12Y 301/03033; C12N 2800/101; C12N 2830/002

(57) ABSTRACT

Aspects of the disclosure relate to production of vaccinia capping enzyme (VCE) in host cells. For example, host cells may comprise: a promoter; a ribosome binding site (RBS); a nucleic acid encoding a vaccinia capping enzyme (VCE) or VCE subunit; and a terminator.

### CROSS REFERENCE TO RELATED APPLICATIONS

This application claims the benefit under 35 U.S.C. § 119(e) of U.S. Provisional Application No. 63/167,249 filed Mar. 29, 2021, entitled “PRODUCTION OF VACCINIA CAPPING ENZYME,” and U.S. Provisional Application No. 63/188,977 filed May 14, 2021, entitled “PRODUCTION OF VACCINIA CAPPING ENZYME,” the entire disclosure of each of which is hereby incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Mar. 29, 2022, is named G091970072WO00-SEQ-OMJ.txt and is 138,941 bytes in size.

### FIELD OF INVENTION

The present disclosure relates to nucleic acids, cells, and methods useful for the production of vaccinia capping enzyme.

### BACKGROUND

The 7-methylguanylate cap structure (m7G cap 0) plays an essential role in cap-dependent initiation of protein synthesis and is involved in stabilization, transport, and translation of eukaryotic messenger RNA (mRNA). Vaccinia capping enzyme (VCE), an enzyme from the vaccinia virus, is efficient at adding the m7G cap 0 to the 5′end of RNA, thereby improving RNA stability and translational competence. VCE can be useful for the production of mRNAs. However, difficulty with expressing and producing VCE at scale has previously been reported.

### SUMMARY

Increased production of VCE would be useful to meet increasing demand for this enzyme. Increased production of VCE may be particularly useful in the production of mRNA vaccines. Aspects of the present disclosure provide non-naturally occurring nucleic acids, cells, and methods useful for the production of VCE.

Aspects of the disclosure relate to non-naturally occurring nucleic acids comprising: (a) a promoter, wherein the promoter comprises a sequence that is at least 90% identical to SEQ ID NO: 8 or 9; and (b) a nucleic acid encoding an amino acid sequence that is at least 90% identical to SEQ ID NO: 6 or 29, and/or a nucleic acid encoding an amino acid sequence that is at least 90% identical to SEQ ID NO: 7 or 31, wherein (a) and (b) are operably linked, and wherein the non-naturally occurring nucleic acid further comprises a ribosome binding site (RBS).

In some embodiments, the promoter is inducible by lactose and/or galactose.

In some embodiments, the non-naturally occurring nucleic acid further comprises a terminator. In some embodiments, the RBS comprises a sequence that is at least 90% identical to SEQ ID NO: 10, 11, 12, 13, 14, 15, 16, 17, 37, 38, or 45 and/or the terminator comprises a sequence that is at least 90% identical to SEQ ID NO: 18, 19, or 20.

In some embodiments, the nucleic acid encoding the amino acid sequence that is at least 90% identical to SEQ ID NO: 6 or 29 comprises a nucleic acid sequence that is at least 90% identical to SEQ ID NO: 2, 3, 33 or 34; and/or the nucleic acid encoding the amino acid sequence that is at least 90% identical to SEQ ID NO: 7 or 31 comprises a nucleic acid sequence that is at least 90% identical to SEQ ID NO: 4, 5, 35 or 36.

In some embodiments, the promoter, RBS, and terminator are operably linked to the nucleic acid encoding an amino acid sequence that is at least 90% identical to SEQ ID NO: 6 or 29, and/or the nucleic acid encoding an amino acid sequence that is at least 90% identical to SEQ ID NO: 7 or 31. In some embodiments, the nucleic acid encoding an amino acid sequence that is at least 90% identical to SEQ ID NO: 6 or 29 encodes the amino acid sequence of SEQ ID NO: 6 or 29. In some embodiments, the nucleic acid encoding an amino acid sequence that is at least 90% identical to SEQ ID NO: 7 or 31 encodes the amino acid sequence of SEQ ID NO: 7 or 31. In some embodiments, the nucleic acid encoding an amino acid sequence that is at least 90% identical to SEQ ID NO: 6 or 29 and/or the nucleic acid encoding an amino acid sequence that is at least 90% identical to SEQ ID NO: 7 or 31 encodes the amino acid sequence of SEQ ID NO: 6 or 29 and also encodes the amino acid sequence of SEQ ID NO: 7 or 31.

Further aspects of the disclosure relate to non-naturally occurring nucleic acids comprising: (a) a first promoter, wherein the first promoter comprises a sequence that is at least 90% identical to SEQ ID NO: 8 or 9; (b) a first nucleic acid, wherein the first nucleic acid encodes an amino acid sequence that is at least 90% identical to SEQ ID NO: 6 or 29; (c) a second promoter, wherein the second promoter comprises a sequence that is at least 90% identical to SEQ ID NO: 8 or 9; and (d) a second nucleic acid, wherein the second nucleic acid encodes an amino acid sequence that is at least 90% identical to SEQ ID NO: 7 or 31, wherein (a) and (b) are operably linked, and wherein (c) and (d) are operably linked, and wherein the non-naturally occurring nucleic acid further comprises at least one ribosome binding site (RBS).

In some embodiments, the first promoter and/or the second promoter is inducible by lactose and/or galactose.

In some embodiments, the non-naturally occurring nucleic acid further comprises at least one terminator. In some embodiments, the RBS comprises a sequence that is at least 90% identical to SEQ ID NO: 10, 11, 12, 13, 14, 15, 16, 17, 37, 38, or 45 and/or the terminator comprises a sequence that is at least 90% identical to SEQ ID NO: 18, 19, or 20. In some embodiments, the first nucleic acid comprises a sequence that is at least 90% identical to SEQ ID NO: 2, 3, 33 or 34; and/or the second nucleic acid comprises a sequence that is at least 90% identical to SEQ ID NO: 4, 5, 35 or 36. In some embodiments, the non-naturally occurring nucleic acid comprises a sequence that is at least 90% identical to any one of SEQ ID NO: 21-28, or 49-54.

Further aspects of the disclosure relate to non-naturally occurring nucleic acids comprising a sequence that is at least 90% identical to any one of SEQ ID NOs: 21-28 or 49-54. In some embodiments, the non-naturally occurring nucleic acid does not encode a fusion protein.

Further aspects of the disclosure relate to host cells comprising any of the non-naturally occurring nucleic acids associated with the disclosure. In some embodiments, the non-naturally occurring nucleic acid is integrated into the genome of the host cell in whole or in part. In some embodiments, the non-naturally occurring nucleic acid is expressed on a plasmid.

Further aspects of the disclosure relate to host cells comprising one or more non-naturally occurring nucleic acids comprising: a promoter, wherein the promoter comprises a sequence that is at least 90% identical to SEQ ID NO: 8 or 9, and a nucleic acid encoding an amino acid sequence that is at least 90% identical to SEQ ID NO: 6 or 29 and/or a nucleic acid encoding an amino acid sequence that is at least 90% identical to SEQ ID NO: 7 or 31, wherein one or more of the non-naturally occurring nucleic acids further comprise a ribosome binding site (RBS).

In some embodiments, the promoter is inducible by lactose and/or galactose.

In some embodiments, the RBS comprises a sequence that is at least 90% identical to one of SEQ ID NOs: 10-17, 37, 38, or 45. In some embodiments, one or more of the non-naturally occurring nucleic acids further comprises a terminator. In some embodiments, one or more of the non-naturally occurring nucleic acids is integrated into the genome of the host cell. In some embodiments, one or more of the non-naturally occurring nucleic acids is expressed on a plasmid.

In some embodiments, the host cell is a bacterial cell. In some embodiments, the bacterial cell is an E. coli cell. In some embodiments, one or more of the nucleic acid sequences encodes an amino acid sequence of SEQ ID NO: 6 or 29. In some embodiments, one or more of the nucleic acid sequences encodes an amino acid sequence of SEQ ID NO: 7 or 31. In some embodiments, one or more of the nucleic acids encodes an amino acid sequence of SEQ ID NO: 6 or 29 and also encodes an amino acid sequence of SEQ ID NO: 7 or 31.

Aspects of the disclosure relate to host cells comprising one or more non-naturally occurring nucleic acids comprising: (a) a first promoter, wherein the first promoter comprises a sequence that is at least 90% identical to SEQ ID NO: 8 or 9; (b) a first nucleic acid, wherein the first nucleic acid encodes an amino acid sequence that is at least 90% identical to SEQ ID NO: 6 or 29; (c) a second promoter, wherein the second promoter comprises a sequence that is at least 90% identical to SEQ ID NO: 8 or 9; and (d) a second nucleic acid, wherein the second nucleic acid encodes an amino acid sequence that is at least 90% identical to SEQ ID NO: 7 or 31, wherein (a) and (b) are operably linked, wherein (c) and (d) are operably linked, and wherein one or more of the non-naturally occurring nucleic acids further comprises at least one ribosome binding site (RBS).

In some embodiments, the promoter is inducible by lactose and/or galactose. In some embodiments, one or more of the non-naturally occurring nucleic acids further comprises at least one terminator. In some embodiments, the RBS comprises a sequence that is at least 90% identical to SEQ ID NO: 10, 11, 12, 13, 14, 15, 16, 17, 37, 38, or 45 and/or the terminator comprises a sequence that is at least 90% identical to SEQ ID NO: 18, 19, or 20.

In some embodiments, the first nucleic acid comprises a sequence that is at least 90% identical to SEQ ID NO: 2, 3, 33 or 34 and/or the second nucleic acid comprises a sequence that is at least 90% identical to SEQ ID NO: 4, 5, 35 or 36. In some embodiments, one or more of the non-naturally occurring nucleic acids comprises a sequence that is at least 90% identical to any one of SEQ ID NO: 21-28 or 49-54.

In some embodiments, the host cell is capable of producing at least 1-fold, 2-fold, 3-fold, 4-fold or 5-fold more vaccinia capping enzyme as compared to a control host cell, wherein the control host cell is a wildtype E. coli cell. In some embodiments, the host cell is capable of producing at least 50 mg/L, 100 mg/L, 150 mg/L, 200 mg/L, 250 mg/L, 300 mg/L, 350 mg/L, 400 mg/L, or 450 mg/L vaccinia capping enzyme. In some embodiments, the non-naturally occurring nucleic acid does not encode a fusion protein.

Further aspects of the disclosure relate to methods of producing vaccinia capping enzyme comprising culturing any of the host cells of the disclosure. In some embodiments, the method further comprises purification of the vaccinia capping enzyme.

Further aspects of the disclosure relate to non-naturally occurring nucleic acids comprising: (a) a promoter, wherein the promoter is a Ptac promoter or a functional fragment thereof, or a P(T5) 2xlacO promoter or a functional fragment thereof; and (b) a nucleic acid encoding a D1 subunit of VCE and/or a D12 subunit of vaccinia capping enzyme, wherein (a) and (b) are operably linked, and wherein the non-naturally occurring nucleic acid further comprises a ribosome binding site (RBS).

In some embodiments, the promoter is inducible by lactose and/or galactose. In some embodiments, the non-naturally occurring nucleic acid does not encode a fusion protein.

In some embodiments, the host cell has increased expression of ftsZ relative to a wildtype cell. In some embodiments, the host cell expresses one or more copies of ftsZ on one or more plasmids. In some embodiments, one or more copies of ftsZ are integrated into the genome of the host cell in whole or in part.

In some embodiments, the host cell has increased expression of metK relative to a wildtype cell. In some embodiments, the host cell expresses one or more copies of metK on one or more plasmids. In some embodiments, one or more copies of metK are integrated into the genome of the host cell in whole or in part.

In some embodiments, the host cell has increased expression of mreB relative to a wildtype cell. In some embodiments, the host cell expresses one or more copies of mreB on one or more plasmids. In some embodiments, one or more copies of mreB are integrated into the genome of the host cell in whole or in part.

In some embodiments, the host cell is cultured in the presence of SAM- and GTP-related metabolites.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items. The term “a” or “an” refers to one or more of an entity.

### BRIEF DESCRIPTION OF DRAWINGS

The accompanying drawings are not intended to be drawn to scale. For purposes of clarity, not every component may be labeled in every drawing. In the drawings:

FIG. 1A-1B provides a schematic showing the generation of mRNA Cap 0 structure by VCE. FIG. 1A depicts the generation of RNA from plasmid DNA followed by VCE capping. FIG. 1B depicts the capping reactions catalyzed by VCE to generate mRNA m7GpppG (Cap 0).

FIG. 2 depicts a graph showing the maximum soluble enzyme titers from fed batch fermentation of the top 23 E. coli candidate VCE production strains. Positive control strain t778543 was derived from the expression system of Fuchs et al. (2016) RNA 22:1454-1466.

FIG. 3 depicts a graph showing the soluble enzyme titers from a 50-hour fed batch fermentation of the top 8 E. coli candidate VCE production strains (816008, 816072, 816070, 816056, 807172, 807173, 815995, and 815917). The time course data show the plotting of 3 bioreactor replicates with error bars showing analytical variance across 4 lysis bioreplicates.

FIG. 4 depicts a graph showing the soluble enzyme titers from a 50-hour fed batch fermentation for 6 E. coli candidate VCE production strains (807175, 807176, 815930, 815934, 816019, and 816020) with no inducer, and 1 E. coli candidate VCE production strain (870868) induced by IPTG, lactose, galactose, and no inducer. The time course data show the plotting of 2 bioreactor replications with error bars showing analytical variance across 2 lysis bioreplicates.

### DETAILED DESCRIPTION

The present disclosure provides, in some aspects, host cells that are engineered for production of VCE. These engineered host cells express recoded nucleic acids encoding the VCE subunits D1 and/or D12 under the control of synthetic promoters. Difficulties expressing and producing VCE at scale have previously been reported. It is surprisingly demonstrated in the Examples of this disclosure that host cells comprising optimized combinations of genetic elements, such as synthetic promoters, ribosomal binding sites (RBSs), recoded nucleic acid sequences, and terminators, produced increased levels of VCE relative to control host cells. Host cells described in this application may be used to produce VCE at increased titers compared with past approaches.

### Vaccinia Capping Enzyme

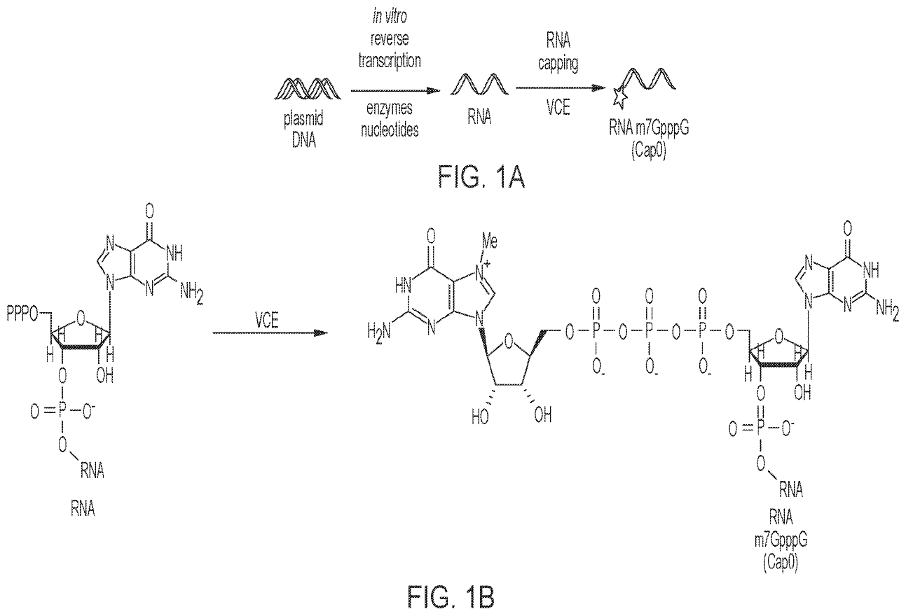

Vaccinia Capping Enzyme (VCE) is a heterodimeric RNA capping enzyme encoded by the vaccinia virus and consisting of two subunits, the large subunit D1 and the small subunit D12. The large subunit D1 comprises three enzymatic activities: 1) RNA triphosphatase; 2) guanylyltransferase; and 3) guanine methyltransferase, all of which are necessary for the enzymatic addition of a complete Cap 0 structure m7Gppp5′N to 5′ triphosphate RNA (FIG. 1B). The guanine methyltransferase activity of the large subunit D1 requires association with the small subunit D12 to function efficiently. Aspects of mRNA capping are described in, and incorporated by reference, from Ramanathan et al. (2016). Nucleic Acids Res. (16): 7511-7526. As described in the Examples section of this application, overexpression of recoded nucleic acids encoding D1 and/or D12 under the control of various combinations of synthetic promoters, RBSs, and terminators surprisingly improved the productivity and yield of VCE-producing strains. Without wishing to be bound by any theory, the recoded nucleic acids encoding D1 and/or D12 provided in this disclosure, expressed under the control of specific combinations of synthetic promoters, RBSs, and/or terminators described in this disclosure, may provide an improved balance of D1:D12 co-expression, including sufficient expression of D12, which may lead to improved stabilization of the D1 subunit, resulting in increased yields of VCE.

The amino acid sequence of the VCE D1 subunit corresponds to UniProt Accession Number P04298 and is provided by SEQ ID NO: 29. In some embodiments, the sequence of a VCE D1 subunit associated with the disclosure comprises SEQ ID NO: 29 or a conservatively substituted version thereof. In some embodiments, the sequence of a VCE D1 subunit associated with the disclosure contains a tag. In some embodiments, the sequence of a VCE D1 subunit associated with the disclosure comprises SEQ ID NO: 6 or a conservatively substituted version thereof. In some embodiments, a VCE D1 subunit associated with the disclosure comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 29 or 6, or a conservatively substituted version thereof; or a VCE D1 subunit sequence otherwise described in this application or known in the art.

The VCE D1 subunit is encoded by the gene VACWR106 (SEQ ID NO: 30). In some embodiments, a nucleic acid encoding D1 comprises SEQ ID NO: 30. In other embodiments, a nucleic acid encoding D1 is recoded. In some embodiments, a nucleic acid encoding D1 comprises SEQ ID NO: 2, 3, 30, 33 or 34. In some embodiments, a nucleic acid encoding D1 comprises a sequence that is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 2, 3, 30, 33 or 34; a D1 recoded sequence within Table 3; or a sequence encoding D1 otherwise described in this application or known in the art.

The amino acid sequence of the VCE D12 subunit corresponds to UniProt Accession number P04318 and is provided by SEQ ID NO: 31. In some embodiments, the sequence of a VCE D12 subunit associated with the disclosure comprises SEQ ID NO: 31 or a conservatively substituted version thereof. In some embodiments, the sequence of a VCE D12 subunit associated with the disclosure contains a tag. In some embodiments, the sequence of a VCE D12 subunit associated with the disclosure comprises SEQ ID NO: 7 or a conservatively substituted version thereof. In some embodiments, a VCE D12 subunit associated with the disclosure comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 31 or 7 or a conservatively substituted version thereof; or a VCE D12 subunit sequence otherwise described in this application or known in the art.

The VCE D12 subunit is encoded by the gene VACWRI 17 (SEQ ID NO: 32). In some embodiments, a nucleic acid encoding D12 comprises SEQ ID NO: 32. In other embodiments, a nucleic acid encoding D12 is recoded. In some embodiments, a nucleic acid encoding D12 comprises SEQ ID NO: 4, 5, 32, 35 or 36. In some embodiments, a nucleic acid encoding D12 comprises a sequence that is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 4, 5, 32, 35 or 36; a D12 recoded sequence within Table 3; or a sequence encoding D12 otherwise described in this application or known in the art.

A host cell described in this application can comprise a VCE or VCE subunit and/or a nucleic acid encoding such an enzyme or enzyme subunit. In some embodiments, a host cell comprises a nucleic acid encoding a VCE that comprises the amino acid sequence of SEQ ID NO: 6 or 29 and/or a nucleic acid encoding a VCE that comprises the amino acid sequence of SEQ ID NO 7 or 31; or a VCE otherwise described in this application or known in the art. In some embodiments, a host cell comprises a nucleic acid encoding a VCE D1 subunit that comprises the sequence of SEQ ID NO: 6 or 29; or a VCE D1 subunit otherwise described in this application or known in the art. In some embodiments, a host cell comprises a nucleic acid encoding a VCE D12 subunit that comprises the sequence of SEQ ID NO: 7 or 31; or a VCE D12 subunit otherwise described in this application or known in the art. In some embodiments, a host cell comprises a nucleic acid that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 2, 3, 4, 5, 30, 32, 33, 34, 35 or 36; a nucleic acid encoding a VCE or VCE subunit in Table 3; or a nucleic acid encoding a VCE or VCE subunit otherwise described in this application or known in the art.

In some embodiments, the large and small subunits (D1 and D12) of VCE are transcribed on separate mRNAs. The mRNAs can be expressed on one or more plasmids in a host cell or integrated into the genome of a host cell. In some embodiments, a nucleic acid encodes only one subunit (e.g., encodes only D1 or only D12). In some embodiments, a nucleic acid encoding D1 or D12 is expressed on a plasmid. In some embodiments, a nucleic acid encoding D1 or D12 is integrated into the chromosome of a cell.

In some embodiments, the large and small subunits (D1 and D12) of VCE are transcribed together as a single polycistronic mRNA wherein the same regulatory sequence (e.g., promoter) controls the expression of both VCE subunits (D1 and D12). The mRNA encoding both subunits can be expressed on a plasmid in a host cell or integrated into the genome of a host cell. In some embodiments, a nucleic acid encoding D1 and D12 is expressed on a plasmid. In some embodiments, a nucleic acid encoding D1 and D12 is integrated into the chromosome of a cell.

In some embodiments, the large and small subunits (D1 and D12) of VCE are transcribed from the same mRNA within two monocistronic units, whereby the expression of each subunit (D1 and D12) is under the control of its own regulatory sequences (e.g., its own promoter). The mRNA encoding both monocistronic units can be expressed on a plasmid in a host cell or integrated into the genome of a host cell. In some embodiments, the nucleic acid is expressed on a plasmid. In some embodiments, the nucleic acid is integrated into the chromosome of a cell.

In some embodiments, a host cell comprises 2 or more copies of a nucleic acid encoding a VCE or one or more VCE subunits (D1 and/or D12). In some embodiments, a host cell comprises 3 or more, 4 or more, 5 or more, 6 or more, 7 or more, 8 or more, 9 or more, or 10 or more copies of a nucleic acid encoding a VCE or one or more VCE subunits (D1 and/or D12).

In some embodiments in which a nucleic acid encodes both D1 and D12, the portion of the nucleic acid that comprises a sequence encoding D1 is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 2, 3, 30, 33, or 34; a D1 recoded sequence within Table 3; or a sequence encoding D1 otherwise described in this application or known in the art.

In some embodiments in which a nucleic acid encodes both D1 and D12, the portion of the nucleic acid that comprises a sequence encoding D12 is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 4, 5, 32, 35, or 36; a D12 recoded sequence within Table 3; or a sequence encoding D12 otherwise described in this application or known in the art.

In some embodiments, nucleic acids of the disclosure do not encode a fusion protein comprising the D1 and D12 subunits.

In other embodiments, nucleic acids of the disclosure may encode a fusion protein comprising the D1 and D12 subunits. A fusion protein comprising the D1 and D12 subunits can include a cleavage site between the D1 and D12 subunits. In some embodiments in which a nucleic acid encodes both D1 and D12, the nucleic acid encodes an amino acid sequence which includes a cleavage site between the sequence encoding D1 and the sequence encoding D12. In some embodiments the cleavage site is a TEV cleavage site.

Aspects of the disclosure relate to host cells that express heterologous nucleic acids encoding a VCE or VCE subunit (D1 and/or D12). It should be appreciated that any mechanism or combination of mechanisms for increasing expression of a nucleic acid encoding a VCE or VCE subunit (D1 and/or D12) is contemplated by the disclosure. For example, a host cell may have increased copy number of a nucleic acid encoding a VCE or VCE subunit (D1 and/or D12), and/or one or more copies of the nucleic acid may be regulated by strong promoters that increase the expression of the nucleic acid relative to its native promoter. In some embodiments, increased copy number of a nucleic acid encoding a VCE or VCE subunit (D1 and/or D12), is achieved by expressing one or more copies on one or more plasmids. In other embodiments, increased copy number of a nucleic acid encoding a VCE or VCE subunit (D1 and/or D12), is achieved by integrating one or more copies of the nucleic acid into the chromosome.

### Regulation of Expression of Genes Associated with the Disclosure

The present disclosure encompasses methods comprising heterologous expression of nucleic acids in a host cell. The term “heterologous” with respect to a nucleic acid, such as a nucleic acid comprising a gene, or a nucleic acid comprising a regulatory region such as a promoter or ribosome binding site, is used interchangeably with the term “exogenous” and the term “recombinant” and refers to: a nucleic acid that has been artificially supplied to a biological system; a nucleic acid that has been modified within a biological system; or a nucleic acid whose expression or regulation has been manipulated within a biological system. A heterologous nucleic acid that is introduced into or expressed in a host cell may be a nucleic acid that comes from a different organism or species than the host cell, or may be a synthetic nucleic acid, or may be a nucleic acid that is also endogenously expressed in the same organism or species as the host cell. For example, a nucleic acid that is endogenously expressed in a host cell may be considered heterologous when it is: situated non-naturally in the host cell; expressed recombinantly in the host cell, either stably or transiently; modified within the host cell; selectively edited within the host cell; expressed in a non-natural copy number within the host cell; or expressed in a non-natural way within the host cell, such as by manipulating regulatory regions that control expression of the nucleic acid. In some embodiments, a heterologous nucleic acid is a nucleic acid that is endogenously expressed in a host cell but whose expression is driven by a promoter that does not naturally regulate expression of the nucleic acid. In other embodiments, a heterologous nucleic acid is a nucleic acid that is endogenously expressed in a host cell and whose expression is driven by a promoter that does naturally regulate expression of the nucleic acid, but the promoter or another regulatory region is modified. In some embodiments, the promoter is recombinantly activated or repressed. For example, gene-editing based techniques may be used to regulate expression of a nucleic acid, including an endogenous nucleic acid, from a promoter, including an endogenous promoter. See, e.g., Chavez et al., Nat Methods. 2016 Jul; 13(7): 563-567. A heterologous nucleic acid may comprise a wild-type sequence or a mutant sequence as compared with a reference nucleic acid sequence.

In some embodiments, a nucleic acid encoding any of the proteins described in this application is under the control of one or more regulatory sequences. A regulatory sequence, as used in this disclosure, refers to a nucleic acid sequence that can influence or control (e.g., increase or decrease) the expression of a coding sequence (e.g., a gene). In some embodiments, a regulatory sequence may include one or more of a promoter, ribosome binding site, enhancer, silencer and/or terminator.

In some embodiments, a nucleic acid is expressed under the control of a promoter. In some embodiments, a promoter is heterologous. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context. In some embodiments, a different promoter has increased strength relative to a native promoter, e.g., the stronger promoter leads to increased expression of a gene relative to regulation of the gene by its native promoter. One of ordinary skill in the art would understand how to assess promoter strength based on methods known in the art. Aspects of the disclosure relate to expression of nucleic acids encoding one or both subunits of VCE under the control of synthetic promoters.

In some embodiments, the promoter is a synthetic promoter. As used in this application, a “synthetic promoter” refers to a promoter that is not known to occur in nature. As demonstrated in the Examples, expression of nucleic acids encoding D1 and/or D12 VCE subunits under the control of synthetic promoters was effective in increasing production of VCE.

In some embodiments, the promoter that drives expression of nucleic acids encoding the D1 and/or D12 VCE subunit comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 8 (Ptac). In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 8. In some embodiments, the promoter that drives expression of nucleic acids encoding the D1 and/or D12 VCE subunit comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 9 (P(T5) 2xlacO). In some embodiments, the promoter comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 9.

In some embodiments, the promoter is Ptac or a functional fragment thereof, or P(T5) 2xlacO or a functional fragment thereof. A fragment of a nucleic acid refers to a portion up to but not including the full-length nucleic acid molecule. A functional fragment of a nucleic acid of the disclosure refers to a biologically active portion of a nucleic acid. A biologically active portion of a genetic regulatory element such as a promoter may comprise a portion or fragment of a full length genetic regulatory element and have the same type of activity as the full length genetic regulatory element, although the level of activity of the biologically active portion of the genetic regulatory element may vary compared to the level of activity of the full length genetic regulatory element.

Other non-limiting examples of synthetic promoters include: P_{(Bba_j23104)}; P_{(galP)}; P_{(apFAB322)}; P_{(apFAB29)}; P_{(apFAB76)}; P_{(apFAB339)}; P_{(apFAB346)}; P_{(apFAB101)}; P_{(gcvTp)}; CP38, CP44, osmY, apFAB38, xthA, poxB, lacUV5, pLlacO1, pLTetO1, apFAB56, Trc, apFAB45, apFAB70, apFAB71, apFAB92, T7A1, bad, and rha.

In some embodiments, the promoter that drives expression of the genes encoding the VCE D1 and/or D12 subunits in a naturally occurring vaccinia virus is used to drive expression of one or more heterologous nucleic acids encoding the VCE D1 and/or D12 subunits.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1,TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include P_{bad}, P_{mgrB}, P_{trc2}, P_{lac/ara}, CP6, CP25, CP38, CP44, CP43, CP31, CP24, CP18, CP27, CP37, CP17, CP2, CP4, CP45, CP1, CP22, CP19, CP34, CP20, CP11, CP26, CP3, CP14, CP13, CP40, CP8, CP28, CP10, CP32, CP30, CP9, CP46, CP23, CP39, CP35, CP33, CP15, CP29, CP12, CP41, CP16, CP42, CP7, Pm, P_{H207}, P_{D/E20}, P_{N25}, P_{G25}, P_{J5}, P_{A1}, P_{A2}, P_{L}, P_{lac}, P_{lacUV5}, P_{tacI}, and P_{con}. Prokaryotic promoters are further described in, and incorporated by reference from Jensen et al. (1998) Appl Environ Microbiol. 64:82-7, Kosuri et al. (2013) Proc Natl Acad Sci U S A. 110:14024-9, and Deuschle et al. (1986) EMBO J. 5:2987-94.

In some embodiments, the promoter is an inducible promoter. As used in this application, an “inducible promoter” is a promoter controlled by the presence or absence of a molecule. This may be used, for example, to controllably induce the expression of an enzyme. Non-limiting examples of inducible promoters include chemically regulated promoters and physically regulated promoters. For chemically regulated promoters, the transcriptional activity can be regulated by one or more compounds, such as alcohol, tetracycline, lactose, galactose, a steroid, a metal, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein ((TA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a lactose-inducible promoter. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination.

In some embodiments, the inducer is isopropyl β-d-1-thiogalactopyranoside (IPTG). In some embodiments, the inducer is vanillic acid. In some embodiments, the inducer is cuminic acid. In some embodiments, the inducer is anhydrotetracycline.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1, ADH1, ADH2, ENO2, and SOD1.

Other inducible promoters or constitutive promoters, including synthetic promoters, that may be known to one of ordinary skill in the art are also contemplated. In some embodiments, synthetic promoters encompassed by the disclosure have increased strength relative to native promoters.

Translation of a VCE and/or VCE subunits can be enhanced, at least in part, by the presence of an RBS. Used in this application, an “RBS” or “ribosome binding site” refers to a regulatory sequence upstream of a start codon in an mRNA that is involved with recruitment of ribosomes. In some embodiments, an RBS is heterologous. Host cells can express a native RBS, e.g., the RBS in its endogenous context, which provides normal regulation of expression of a gene or operon. Alternatively, an RBS may be an RBS that is different from a native RBS associated with a gene, e.g., the RBS is different from the RBS of a gene in its endogenous context. An RBS can be synthetic. As used in this application, a “synthetic RBS” refers to an RBS that is not known to occur in nature. Synthetic RBSs are further described in, and incorporated by reference from, Salis et al. (2009) Nat. Biotechnol. 27, 946-950 (2009).

In some embodiments, the RBS comprises a sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NOs: 10-17, 37, 38, and 45. In some embodiments, the RBS comprises no more than 1, 2, 3, 4, 5, 6, 7, 8, 9, 10, 11, 12, 13, 14, 15, 16. 17, 18, 19, 20, 21, 22, 23, 24, 25, 26, 27, or 28 nucleotide substitutions, insertions, additions, or deletions relative to SEQ ID NOs: 10-17, 37, 38, and 45.

In some embodiments, the RBS is apFAB873, apFAB826, DeadRBS, apFAB871, BBa_J61133, BBa_J61139, apFAB843, BBa_J61124, apFAB864, apFAB964, BBa_J61101, BBa_J61131, salis-3-11, BBa_J61125, BBa_J61118, apFAB922, BBa_J61130, BBa_J61134, BBa_J61128, BBa_J61107, apFAB869, apFAB890, BBa_J61120, BBa_J61109, BBa_J61103, apFAB868, apFAB914, BBa_J61119, BBa_J61126, B0032_RBS, apFAB895, BBa_J61136, apFAB866, GSGV_RBS, apFAB918, BBa_J61129, apFAB867, apFAB903, apFAB872, BBa_J61137, BBa_J61111, apFAB821, apFAB844, BBa_J61110, BBa_J61112, BBa_J61104, BBa_J61122, apFAB854, BBa_J61127, BBa_J61113, GSG_RBS, apFAB892, BBa_J61115, apFAB927, BBa_J61108, Anderson_RBS, apFAB883, apFAB894, BBa_J61132, apFAB860, BBa_J61100, apFAB856, apFAB862, apFAB865, BBa_J61106, apFAB845, apFAB820, apFAB954, apFAB910, salis-4-10, apFAB901, salis-4-4, apFAB832, apFAB909, salis-4-7, apFAB861, apFAB876, apFAB827, salis-2-4, Alon_RBS, apFAB831, apFAB857, apFAB863, apFAB912, apFAB889, apFAB851, apFAB884, apFAB833, apFAB848, apFAB839, salis-1-21, apFAB923, Plotkin_RBS, apFAB842, salis-2-3, apFAB837, apFAB916, apFAB834, apFAB904, apFAB917, salis-1-10, Invitrogen_RBS, salis-1-1, salis-1-3, salis-3-3, salis-4-2, JBEI_RBS, salis-1-5, B0034_RBS, B0030_RBS, or Bujard_RBS, which are further described in and incorporporated by reference from Kosuri et al. (2013) Proc Natl Acad Sci U S A. 110:14024-9. In certain embodiments, the RBS is apFAB873 or apFAB826.

Nucleic acids associated with the disclosure may comprise a terminator (e.g., a transcriptional terminator located downstream or 3′ to the portion of the nucleic acid encoding VCE or a subunit thereof). In some embodiments, the terminator comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 18. In some embodiments, the terminator comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 18. In some embodiments, the terminator comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 19. In some embodiments, the terminator comprises not more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 19. In some embodiments, the terminator comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 20. In some embodiments, the terminator comprises not more than 1, 2, 3, 4, 5, 6, 7, 8, 9, 10, 11, 12, 13, 14, 15, 16, 17, 18, 19. 20, 21, 22, 23, 24, 25, 26, 27, 28, 29, 30, 31, 32, 33, 34, 35, 36, 37, 38, 39, or 40 nucleotide substitutions, insertions, additions, or deletions relative to SEQ ID NO: 20.

Expression of VCE and/or VCE subunits can also be increased, at least in part, by the presence of an enhancer.

A coding sequence and a regulatory sequence are said to be “operably joined” or “operably linked” when the coding sequence and the regulatory sequence are covalently linked and/or the expression or transcription of the coding sequence is under the influence or control of the regulatory sequence. In some embodiments, a promoter, such as Ptac or a functional fragment thereof, or P(T5) 2xlacO or a functional fragment thereof, is operably linked to one or more nucleic acids encoding VCE subunit D1 and/or D12. In some embodiments, a promoter, such as Ptac or a functional fragment thereof, or P(T5) 2xlacO or a functional fragment thereof, and one or more RBSs, are operably linked to one or more nucleic acids encoding VCE subunit D1 and/or D12. In some embodiments, a promoter, such as Ptac or a functional fragment thereof, or P(T5) 2xlacO or a functional fragment thereof, and one or more RBSs, are operably linked to one or more nucleic acids encoding VCE subunit D1 and/or D12. In some embodiments, a promoter, such as SEQ ID NO: 8 or 9 or a functional fragment thereof, is operably linked to the one or more nucleic acids encoding VCE subunit D1 and/or D12.

A nucleic acid described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a lactose and/or galactose-inducible or doxycycline-inducible vector). A vector described in this application may be introduced into a suitable host cell using any method known in the art.

In some embodiments, a vector replicates autonomously in the cell. In some embodiments, an autonomously replicating vector comprises an origin of DNA replication; if required by the origin, a gene encoding a replicase and/or other trans-acting factor can be provided on the vector and/or on a host cell chromosome. In some embodiments, an autonomously replicating vector can comprise a cis-acting region required for the vector to be stably maintained in the cell; if required for stable maintenance of the vector, a gene(s) encoding a trans-acting factor(s) can be provided on the vector and/or on a host cell chromosome. In some embodiments, a vector integrates into a chromosome within a cell (e.g., a suicide vector). A vector can contain one or more endonuclease restriction sites that can be cut by a restriction endonuclease to insert and ligate a nucleic acid containing a gene described in this application to produce a recombinant vector that is able to replicate in a cell. Vectors can be composed of DNA or RNA. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector.

In some embodiments, the nucleic acid sequence of a gene described in this application is recoded. As used in this disclosure, a “recoded” nucleic acid sequence refers to a nucleic acid sequence that has been modified with respect to a reference nucleic acid sequence by exchanging one or more codons with a synonymous codon. In some embodiments, the exchange of one or more codons with a synonymous codon is based on selection of codons that are preferentially used by an organism or host cell in which a nucleic acid will be expressed heterologously. Recoding may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not recoded. The choice and design of one or more appropriate vectors suitable for inducing expression of one or more genes in a host cell is within the ability of one of ordinary skill in the art. Expression vectors containing the necessary elements for expression are commercially available and known to one of ordinary skill in the art (sec, e.g., Sambrook et al., Molecular Cloning: A Laboratory Manual, Fourth Edition, Cold Spring Harbor Laboratory Press, 2012).

### Production of VCE

Any of the nucleic acids, proteins, host cells, and methods described in this application may be used for the production of VCE. In general, the term “production” is used to refer to the generation of one or more products (e.g., VCE subunits D1 and/or D12 of interest and/or VCE), for example, from a particular nucleic acid. The amount of production of VCE may be evaluated at any one or more steps of a pathway, such as a final product or an intermediate product, using metrics familiar to one of ordinary skill in the art. Production may be assessed by any metrics known in the art, for example, by assessing volumetric productivity, enzyme kinetics/reaction rate, specific productivity, biomass-specific productivity, titer, yield, and total titer of one or more products (e.g., products of interest and/or by-products/off-products).

In some embodiments, the metric used to measure production may depend on whether a continuous process is being monitored or whether a particular end product is being measured. For example, in some embodiments, metrics used to monitor production by a continuous process may include volumetric productivity, enzyme kinetics and reaction rate. In some embodiments, metrics used to monitor production of a particular product may include specific productivity, biomass-specific productivity, titer, yield, and total titer of one or more products (e.g., products of interest and/or by-products/off-products). The term “volumetric productivity” or “production rate” refers to the amount of product formed per volume of medium per unit of time. Volumetric productivity can be reported in gram per liter per hour (g/L/h).

The term “specific productivity” of a product refers to the rate of formation of the product normalized by unit volume or mass or biomass and has the physical dimension of a quantity of substance per unit time per unit mass or volume [M·T⁻¹·M⁻¹ or M·T⁻¹·L⁻³, where M is mass or moles, T is time, L is length].

The term “biomass specific productivity” refers to the specific productivity in gram product per gram of cell dry weight (CDW) per hour (g/g CDW/h) or in mmol of product per gram of cell dry weight (CDW) per hour (mmol/g CDW/h). Using the relation of CDW to OD600 for the given microorganism, specific productivity can also be expressed as gram product per liter culture medium per optical density of the culture broth at 600 nm (OD) per hour (g/L/h/OD). Also, if the elemental composition of the biomass is known, biomass specific productivity can be expressed in mmol of product per C-mole (carbon mole) of biomass per hour (mmol/C-mol/h).

The term “yield” refers to the amount of product obtained per unit weight of a certain substrate and may be expressed as g product per g substrate (g/g) or moles of product per mole of substrate (mol/mol). Yield may also be expressed as a percentage of the theoretical yield. “Theoretical yield” is defined as the maximum amount of product that can be gencrated per a given amount of substrate as dictated by the stoichiometry of the metabolic pathway used to make the product and may be expressed as g product per g substrate (g/g) or moles of product per mole of substrate (mol/mol).

The term “titer” refers to the strength of a solution or the concentration of a substance in solution. For example, the titer of a product of interest (e.g., small molecule, peptide, synthetic compound, fuel, alcohol, etc.) in a fermentation broth is described as g of product of interest in solution per liter of fermentation broth or cell-free broth (g/L) or as g of product of interest in solution per kg of fermentation broth or cell-free broth (g/Kg).

The term “total titer” refers to the sum of all product of interest produced in a process, including but not limited to the product of interest in solution, the product of interest in gas phase if applicable, and any product of interest removed from the process and recovered relative to the initial volume in the process or the operating volume in the process. For example, the total titer of a product of interest (e.g., small molecule, peptide, synthetic compound, fuel, alcohol, etc.) in a fermentation broth is described as g of product of interest in solution per liter of fermentation broth or cell-free broth (g/L) or as g of product of interest in solution per kg of fermentation broth or cell-free broth (g/Kg).

In some embodiments, host cells described in this application can produce titers of at least 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, or 1600 mg/L of VCE. In some embodiments, host cells described in this application exhibit production rates of at least 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5 mg/L/h for production of VCE. In some embodiments, the titer is approximately 550 mg/L. In some embodiments, the production rate is approximately 10 mg/L/h. In some embodiments, a host cell is capable of producing at least 1-fold, 1.5-fold, 2-fold, 2.5 fold, 3-fold, 3.5 fold, 4-fold, 4.5-fold, 5-fold, or 10-fold more VCE relative to a control host cell. In some embodiments, a control host cell is a cell that does not heterologously express one or more nucleic acids encoding VCE subunit D1 and/or D12. In some embodiments, a control host cell is a wildtype cell, such as a wildtype E. coli cell. In some embodiments, a control host cell comprises the same nucleic acids encoding VCE subunit D1 and/or D12 as a test cell, but comprises different regulatory sequences controlling expression of the one or more nucleic acids encoding VCE subunit D1 and/or D12.

### Additional Cellular Modifications

Production of VCE in a host cell may, in some embodiments, lead to an increase in viscosity and/or a slowing of fermentation. Without wishing to be bound by any theory, these effects may be caused by cell elongation. In some embodiments, expression of one or more genes is increased in a host cell to offset the impact of production of VCE.

In some embodiments, expression of a gene encoding a FtsZ protein is increased in a host cell to offset the impact of production of VCE. The E. coli FtsZ protein is an important regulator of cell size. The FtsZ protein is influenced by levels of S-adenosylmethionine (SAM) and guanosyltriphosphate (GTP) within the cell. Both SAM and GTP are known substrates of VCE. Without wishing to be bound by any theory, VCE overexpression may impede the homeostasis of native ftsZ, resulting in the elongation of cells and an increase in viscosity.

The amino acid sequence of the E. coli FtsZ protein corresponds to UniProt Accession Number P0A9A6 and is provided by SEQ ID NO: 39. In some embodiments, a FtsZ protein associated with the disclosure comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 39, or a conservatively substituted version thereof; or a FtsZ sequence otherwise described in this application or known in the art.

The E. coli FtsZ protein is encoded by a nucleic acid sequence available at GenBank Accession Number CP001509.3, which corresponds to the E. coli BL21(DE3) genome sequence. In some embodiments, a nucleic acid encoding a FtsZ protein comprises the sequence of SEQ ID NO: 42. In some embodiments, a nucleic acid encoding a FtsZ protein is recoded. In some embodiments, a nucleic acid encoding a FtsZ protein comprises a sequence that is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 42, or a FtsZ sequence otherwise described in this application or known in the art.

In some embodiments, a host cell expresses an endogenous copy of the ftsZ gene under the control of its native promoter. In some embodiments, a host cell that expresses an endogenous copy of the ftsZ gene under the control of its native promoter also expresses one or more copies of an additional nucleic acid encoding a FtsZ protein. In some embodiments, the one or more copies of the additional nucleic acid encoding the FtsZ protein are either expressed on a plasmid or integrated into the genome of the host cell. In some embodiments, the one or more copies of the additional nucleic acid encoding the FtsZ protein are expressed under the control of one or more synthetic promoters. Translation of a FtsZ protein, under the control of a native or synthetic promoter, can be enhanced, at least in part, by the presence of an RBS. Aspects of the disclosure relate to host cells that overexpress a gene encoding a FtsZ protein. It should be appreciated that any mechanism for increasing expression of a gene encoding a FtsZ protein is contemplated by the disclosure. For example, a host cell may have increased copy number of a gene encoding a FtsZ protein and/or one or more copies of the gene may be regulated by strong promoters that increase the expression of the gene relative to its native promoter. In some embodiments, increased copy number of a gene encoding a FtsZ protein is achieved by expressing one or more copies on one or more plasmids. In other embodiments, increased copy number of a gene encoding a FtsZ protein is achieved by integrating one or more copies of the gene into the chromosome.

In some embodiments, a host cell that overexpresses a gene encoding a FtsZ protein exhibits reduced cell elongation and/or reduced viscosity relative to a host cell that does not overexpress a gene encoding a FtsZ protein. In some embodiments, a VCE production strain that overexpresses a gene encoding a FtsZ protein exhibits reduced cell elongation and/or reduced viscosity relative to a host cell that does not overexpress a gene encoding a FtsZ protein.

In some embodiments, expression of the metK gene encoding a SAM synthetase is increased in a host cell to offset the impact of production of VCE. The amino acid sequence of the E. coli MetK protein corresponds to UniProt Accession Number P0A817 and is provided by SEQ ID NO: 40. In some embodiments, a MetK protein associated with the disclosure comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 40, or a conservatively substituted version thereof; or a MetK sequence otherwise described in this application or known in the art.

The E. coli MetK protein is encoded by a nucleic acid sequence available at GenBank Accession Number CP001509.3, which corresponds to the E. coli BL21(DE3) genome sequence. In some embodiments, a nucleic acid encoding a MetK protein comprises the sequence of SEQ ID NO: 43. In some embodiments, a nucleic acid encoding a MetK protein is recoded. In some embodiments, a nucleic acid encoding a MetK protein comprises a sequence that is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 43, or a MetK sequence otherwise described in this application or known in the art.

In some embodiments, a host cell expresses an endogenous copy of the metK gene under the control of its native promoter. In some embodiments, a host cell that expresses an endogenous copy of the metK gene under the control of its native promoter also expresses one or more copies of an additional nucleic acid encoding a MetK protein. In some embodiments, the one or more copies of the additional nucleic acid encoding the MetK protein are either expressed on a plasmid or integrated into the genome of the host cell. In some embodiments, the one or more copies of the additional nucleic acid encoding the MetK protein are expressed under the control of one or more synthetic promoters. Translation of a MetK protein, under the control of a native or synthetic promoter, can be enhanced, at least in part, by the presence of an RBS.

Aspects of the disclosure relate to host cells that overexpress a gene encoding a MetK protein. It should be appreciated that any mechanism for increasing expression of a gene encoding a MetK protein is contemplated by the disclosure. For example, a host cell may have increased copy number of a gene encoding a MetK protein and/or one or more copies of the gene may be regulated by strong promoters that increase the expression of the gene relative to its native promoter. In some embodiments, increased copy number of a gene encoding a MetK protein is achieved by expressing one or more copies on one or more plasmids. In other embodiments, increased copy number of a gene encoding a MetK protein is achieved by integrating one or more copies of the gene into the chromosome.

In some embodiments, a host cell that overexpresses a gene encoding a MetK protein exhibits reduced cell elongation and/or reduced viscosity relative to a host cell that does not overexpress a gene encoding a MetK protein. In some embodiments, a VCE production strain that overexpresses a gene encoding a MetK protein exhibits reduced cell elongation and/or reduced viscosity relative to a host cell that does not overexpress a gene encoding a MetK protein.

In some embodiments, expression of the mreB gene is increased in a host cell to offset the impact of production of VCE. The amino acid sequence of the E. coli MreB protein corresponds to UniProt Accession Number P0A9X4 and is provided by SEQ ID NO: 41. In some embodiments, a MreB protein associated with the disclosure comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 41, or a conservatively substituted version thereof; or a MreB sequence otherwise described in this application or known in the art.

The E. coli MreB protein is encoded by a nucleic acid sequence available at GenBank Accession Number CP001509.3, which corresponds to the E. coli BL21(DE3) genome sequence. In some embodiments, a nucleic acid encoding a MreB protein comprises the sequence of SEQ ID NO: 44. In some embodiments, a nucleic acid encoding a MreB protein is recoded. In some embodiments, a nucleic acid encoding a MreB protein comprises a sequence that is at least 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to SEQ ID NO: 44, or a MreB sequence otherwise described in this application or known in the art.

In some embodiments, a host cell expresses an endogenous copy of the mreB gene under the control of its native promoter. In some embodiments, a host cell that expresses an endogenous copy of the mreB gene under the control of its native promoter also expresses one or more copies of an additional nucleic acid encoding a MreB protein. In some embodiments, the one or more copies of the additional nucleic acid encoding the MreB protein are either expressed on a plasmid or integrated into the genome of the host cell. In some embodiments, the one or more copies of the additional nucleic acid encoding the MreB protein are expressed under the control of one or more synthetic promoters. Translation of a MreB protein, under the control of a native or synthetic promoter, can be enhanced, at least in part, by the presence of an RBS.

Aspects of the disclosure relate to host cells that overexpress a gene encoding a MreB protein. It should be appreciated that any mechanism for increasing expression of a gene encoding a MreB protein is contemplated by the disclosure. For example, a host cell may have increased copy number of a gene encoding a MreB protein and/or one or more copies of the gene may be regulated by strong promoters that increase the expression of the gene relative to its native promoter. In some embodiments, increased copy number of a gene encoding a MreB protein is achieved by expressing one or more copies on one or more plasmids. In other embodiments, increased copy number of a gene encoding a MreB protein is achieved by integrating one or more copies of the gene into the chromosome.

In some embodiments, a host cell that overexpresses a gene encoding a MreB protein exhibits reduced cell elongation and/or reduced viscosity relative to a host cell that does not overexpress a gene encoding a MreB protein. In some embodiments, a VCE production strain that overexpresses a gene encoding a MreB protein exhibits reduced cell elongation and/or reduced viscosity relative to a host cell that does not overexpress a gene encoding a MreB protein.

A host cell described in this application may be cultured in conditions supplemented with the addition of S-adenosylmethionine (SAM) and/or guanosyltriphosphate (GTP)- related metabolites to the fermentation broth. SAM- and GTP- related metabolites (e.g., SAM, cysteine, methionine, serine, adenine, guanine, adenosine, and guanosine) are known in the art and contemplated herein. In some embodiments, a host cell cultured in conditions supplemented with the addition of S-adenosylmethionine (SAM) and/or guanosyltriphosphate (GTP)- related metabolites to the fermentation broth exhibits reduced cell elongation and/or reduced viscosity relative to a host cell that is not cultured in conditions supplemented with the addition of S-adenosylmethionine (SAM) and/or guanosyltriphosphate (GTP)- related metabolites to the fermentation broth. In some embodiments, a VCE production strain that is cultured in conditions supplemented with the addition of S-adenosylmethionine (SAM) and/or guanosyltriphosphate (GTP)- related metabolites to the fermentation broth exhibits reduced cell elongation and/or reduced viscosity relative to a VCE production strain that is not cultured in conditions supplemented with the addition of S-adenosylmethionine (SAM) and/or guanosyltriphosphate (GTP)- related metabolites to the fermentation broth.

A host cell described in this application can comprise one or more of FtsZ, MetK, and/or MreB and/or a nucleic acid encoding such a protein. In some embodiments, a host cell comprises a nucleic acid encoding a FtsZ, MetK, and/or MreB protein that comprises the amino acid sequence of SEQ ID NO: 39, 40 and/or 41 and/or a nucleic acid encoding a FtsZ, MetK, and/or MreB. In some embodiments, a host cell overexpresses FtsZ, MetK, and/or MreB relative to a control. In some embodiments, a host cell that overexpresses FtsZ, MetK, and/or MreB has decreased cell elongation, decreased viscosity, and/or decreased toxicity, relative to a control host cell.

### Variants

Aspects of the disclosure relate to nucleic acids, including nucleic acids encoding polypeptides. Variants of nucleic acids and polypeptides described in this application are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” which is used interchangeably in this disclosure with the term “percent identity,” as known in the art, refers to a relationship between the sequences of two polypeptides or polynucleotides, as determined by sequence comparison (alignment). In some embodiments, sequence identity is determined across the entire length of a sequence. In some embodiments, sequence identity is determined over a region (e.g., a stretch of amino acids or nucleic acids, e.g., the sequence spanning an active site) of a sequence. For example, in some embodiments, sequence identity is determined over a region corresponding to at least 30%, at least 40%, at least 50%, at least 60%, at least 70%, at least 80%, at least 90%, at least 95%, or over 100% of the length of the reference sequence.

Identity measures the percent of identical matches between the smaller of two or more sequences with gap alignments (if any) addressed by a particular mathematical model, algorithms, or computer program. Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the percent identity of two polypeptides is determined by aligning the two amino acid sequences, calculating the number of identical amino acids, and dividing by the length of one of the amino acid sequences. In some embodiments, the percent identity of two nucleic acids is determined by aligning the two nucleotide sequences and calculating the number of identical nucleotide and dividing by the length of one of the nucleic acids.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147: 195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct 11;7:539) using default parameters.

As used in this application, a residue (such as a nucleic acid residue or an amino acid residue) in sequence “X” is referred to as corresponding to a position or residue (such as a nucleic acid residue or an amino acid residue) “n” in a different sequence “Y” when the residue in sequence “X” is at the counterpart position of “n” in sequence “Y” when sequences X and Y are aligned using amino acid sequence alignment tools known in the art.

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between) and include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant comprises a domain that shares a secondary structure (e.g., alpha helix, beta sheet) with a reference polypeptide. In some embodiments, a polypeptide variant shares a tertiary structure with a reference polypeptide. As a non-limiting example, a polypeptide variant may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Functional variants of enzymes are encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 Jul;28(3):405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol. Position-specific scoring matrix (PSSM) uses a position weight matrix to identify consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. Sec, e.g., Stormo et al., Nucleic Acids Res. 1982 May 11;10(9):2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and the single-point mutant. The Rosetta energy function calculates this difference as (ΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul 21;63(2):337-346. Doi: 10.1016/j.molcel.2016.06.012.

In some embodiments, a coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72. 73, 74, 75, 76, 77. 78, 79, 80, 81, 82, 83, 84, 85, 86, 87, 88, 89, 90, 91, 92, 93, 94, 95, 96, 97, 98, 99, 100 or more than 100 positions relative to a reference coding sequence. In some embodiments, the coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67. 68, 69, 70, 71, 72. 73. 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a coding sequence do alter the amino acid sequence of the corresponding polypeptide relative to the amino acid sequence of a reference polypeptide. In some embodiments, the one or more mutations alters the amino acid sequence of the polypeptide relative to the amino acid sequence of a reference polypeptide and alters (enhances or reduces) an activity of the polypeptide relative to the reference polypeptide.

The activity (e.g., specific activity) of any of the polypeptides described in this application (e.g., VCE) may be measured using routine methods. As a non-limiting example, a polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a polypeptide coding sequence may result in conservative amino acid substitutions to provide functionally equivalent variants of the foregoing polypeptides, e.g., variants that retain the activities of the polypeptides. Conservative substitutions may not alter the relative charge or size characteristics or functional activity of the protein in which the amino acid substitution is made.

In some instances, an amino acid is characterized by its R group (see, e.g., Table 1). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. As used in this application “conservative substitution” is used interchangeably with “conservative amino acid substitution” and refers to any one of the amino acid substitutions provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 1
Conservative Amino Acid Substitutions

| Original | | Conservative Amino |
|---|---|---|
| Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing approaches, or by insertions, such as insertion of a tag (e.g., a HIS tag or a GFP tag). As used in this disclosure, a “tag” refers to a sequence that is added to a nucleic acid or protein sequence of interest. A tag can be added for a variety of purposes, such as for detection, purification, and/or localization of a nucleic acid or protein of interest. In some embodiments, a linker sequence is inserted between the sequence of the nucleic acid or protein of interest and the sequence of the tag. In some embodiments, a cleavage site is inserted between the sequence of the nucleic acid or protein of interest and the sequence of the tag. In some embodiments the cleavage site is a TEV cleavage site.

Mutations can include, for example, substitutions, deletions, insertions, additions, selective editing, truncation, and translocations, generated by any method known in the art. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104). A gene may also be edited through of the use of gene editing technologies known in the art, such as CRISPR-based technologies. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 Jan;29(1): 18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). Sec, e.g., Yu and Lutz, Trends Biotechnol. 2011 Jan;29(1):18-25.

It should be appreciated that in a protein that has undergone circular permutation, the linear amino acid sequence of the protein would differ from a reference protein that has not undergone circular permutation. However, one of ordinary skill in the art would be able to readily determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr 1;21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

### Host Cells

The disclosed methods and host cells are exemplified with E. coli, but are also applicable to other host cells, as would be understood by one of ordinary skill in the art.

Suitable host cells include, but are not limited to: bacterial cells, yeast cells, algal cells, plant cells, fungal cells, insect cells, and animal cells, including mammalian cells.

In some embodiments, the host cell is a prokaryotic cell. Suitable prokaryotic cells include gram positive, gram negative, and gram-variable bacterial cells. In some nonlimiting embodiments, the host cell is a species of: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas. In some embodiments, the host cell is a Corynebacterium glutamicum cell. In some embodiments, the host cell is a Serratia marcescens cell. In some embodiments, the host cell is an Escherichia coli cell.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z. mobilis, Z. lipolytica).

Suitable yeast host cells include, but are not limited to: Candida, Hansenula, Saccharomyces, Schizosaccharomyces, Pichia, Kluyveromyces, and Yarrowia. In some embodiments, the yeast cell is Escherichia coli, Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia pastoris, Pichia pseudopastoris, Pichia membranifaciens, Komagataella pseudopastoris, Komagataella pastoris, Komagataella kurtzmanii, Komagataella mondaviorum, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, Candida boidinii or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In some embodiments, the host cell is an Ashbya gossypii cell.

In certain embodiments, the host cell is an algal cell such as Chlamydomonas (e.g., C. Reinhardtii) and Phormidium (P. sp. ATCC29409).

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), bovine (including KOP-R, BT and MDBK), equine (including EK), insect cells, for example fall armyworm (including Sf9 and Sf21), silkmoth (including BmN), cabbage looper (including BTI-Tn-5B1-4) and common fruit fly (including Schneider 2), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart.

### Culturing of Host Cells

Any of the cells disclosed in this application can be cultured in media of any type (rich or minimal) and any composition prior to, during, and/or after contact with and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place that involves a living organism, part of a living organism, and/or isolated or purified enzymes. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, rotary cell culture systems, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the host cell is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated.

In some embodiments, the cells of the present disclosure are adapted to produce VCE or VCE subunits in vivo.

### Purification and Further Processing

In some embodiments, any of the methods described in this application may include isolation and/or purification of VCE produced (e.g., produced in a bioreactor). For example, the isolation and/or purification can involve one or more of cell lysis, centrifugation, extraction, column chromatography, distillation, crystallization, and lyophilization.

VCE produced by any of the recombinant cells disclosed in this application, or any of the in vitro methods described in this application, may be identified and extracted using any method known in the art. Mass spectrometry (e.g., LC-MS, GC-MS) is a non-limiting example of a method for identification and may be used to extract a compound of interest.

The present invention is further illustrated by the following Examples, which should not be construed as limiting. The entire contents of all of the references (including literature references, issued patents, published patent applications, and co-pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. Mention of any reference, article, publication, patent, patent publication, and patent application cited in this application is not, and should not be taken as, an acknowledgment or suggestion that they constitute valid prior art or form part of the common general knowledge of a skilled artisan.

### EXAMPLES

In order that the invention described in this application may be more fully understood, the following examples are set forth. The examples described in this application are offered to illustrate the systems and methods provided in this application and are not to be construed as limiting their scope.

### Example 1: Screen to Identify E. coli VCE Production Strains

To investigate whether it was possible to increase production of VCE in host cells, an E. coli BL21(DE3) strain was transformed with VCE-encoding plasmids to generate ˜300 candidate VCE production library strains. Library strains were designed to express VCE from an extrachromosomal plasmid. 13 different promoters, 21 different RBSs, and 3 different terminators were tested in a variety of different combinations for their ability to drive expression of the genes encoding the VCE D1 and D12 subunits (corresponding to amino acid sequences SEQ ID NOs: 6 and 7, respectively).

A plate-based fermentation screen was developed to quantify VCE production from each of the candidate VCE production library strains. Strains were cultured in LB media at 37° C. followed by induction with 500 μM IPTG at an optical density of ˜1. Following induction, strains were fermented at 30° C. for 5 hours followed by quantification of VCE, measured as total VCE protein concentration (μg/L).

The plate-based screen identified multiple candidate VCE production library strains that produced VCE. Based on the plate-based screen, 23 candidate VCE production library strains were elevated to a secondary screen described in Example 2.

### Example 2: Confirmation of Candidate VCE Production Library Strains

23 candidate VCE production library strains identified in Example 1 were re-screened using Ambr 250s fermentations to determine total VCE concentration (mg/L).

Strains were grown in a rich, animal free media overnight at 37° C.while shaking at 250 rpm in a baffled flask. Stationary cultures were used to inoculate miniature bioreactors with a 250 mL volumetric capacity. The reactors were charged with animal free, semi-defined production medium composed of yeast extract, glycerol, salts and minerals, then the reactors were equilibrated with inlet air until desired oxygenation was achieved. Cultures were grown on batch carbon and a nitrogen feed to the desired biomass load, then lactose was added continuously to induce production of VCE. The cultures were continuously fed while maintaining carbon feed rate on an adaptive control loop to maintain an acceptable oxygen uptake rate. At 45-50 h, the culture fermentations were terminated. Biomass samples taken throughout the experiment and at the end of fermentation were lysed and assayed for intracellular VCE titer and activity.

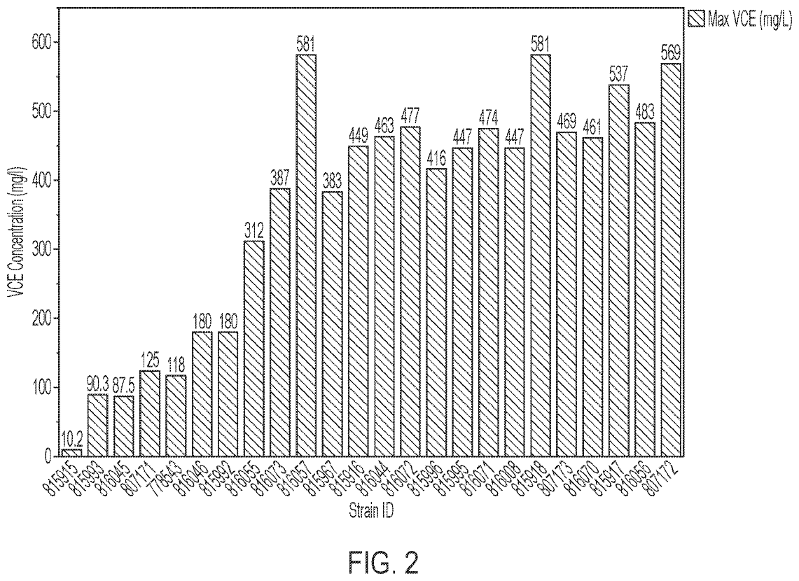

Mean VCE protein concentration (mg/L) produced by each strain is shown in Table 2 and FIG. 2. FIG. 2 depicts the maximum soluble enzyme titers from fed batch fermentation of the top 23 E. coli candidate VCE production library strains in comparison to a positive control strain t778543 derived from the expression system of Fuchs et al. (2016) RNA 22:1454-1466. In Table 2, for each strain, the upper row corresponds to VCE subunit D1 and the lower row corresponds to VCE subunit D12.

TABLE 2
VCE Production Data in Ambr 250s Fermentation System

| | | | | | | Tran- | | | Mean |
|---|---|---|---|---|---|---|---|---|---|
| | | | | | | script | SEQ ID | SEQ ID | VCE |
| | | | | | | shared | NO of | NO of | Protein |
| | | | | | | with | D1 | D12 | Concent |
| Strain | Strain | | | | | VCE- | nucleic | nucleic | ration |
| ID | Type | Promoter | RBS | Inducer | Terminator | D12 | acid | acid | [mg/L] |
| 778543 | Control | P(T7) | T7RBS | IPTG/Lac | | | 2 | | 118 |
| | | P(T7) | T7RBS | IPTG/Lac | pRSF-duet | Yes | | 4 | |
| | | | | | Pre-T7 | | | | |
| | | | | | Terminator | | | | |
| | | | | | Spacer- | | | | |
| | | | | | Terminator, | | | | |
| | | | | | T7 | | | | |
| 807171 | Library | P(T5) | BCD | IPTG/Lac | Bba_J61048 | | 2 | | 125 |
| | | 2xlacO | RBS_ | | | | | | |
| | | | alt1_ | | | | | | |
| | | | BD1 | | | | | | |
| | | P(T7) | T7_RBS | IPTG/Lac | BBa_B0015, | No | | 4 | |
| | | | | | T7 | | | | |
| 807172 | Library | P(T5) | BCD | IPTG/Lac | Bba_J61048 | | 2 | | 569 |
| | | 2xlacO | RBS_ | | | | | | |
| | | | alt1_ | | | | | | |
| | | | BD1 | | | | | | |
| | | Ptac | BCD | IPTG/Lac | Bba_J61048, | No | | 4 | |
| | | | RBS_ | | T7 | | | | |
| | | | alt1_ | | | | | | |
| | | | BD6 | | | | | | |
| 807173 | Library | Ptac | BCD | IPTG/Lac | | | 2 | | 469 |
| | | | RBS_ | | | | | | |
| | | | alt1_ | | | | | | |
| | | | BD10 | | | | | | |
| | | Ptac | BCD | IPTG/Lac | BBa_B0015, | Yes | | 4 | |
| | | | RBS_ | | T7 | | | | |
| | | | alt4_ | | | | | | |
| | | | BD11 | | | | | | |
| 815915 | Library | P(T5) | BCD | IPTG/Lac | Bba_J61048 | | 2 | | 10.2 |
| | | 2xlacO | RBS_ | | | | | | |
| | | | alt1_ | | | | | | |
| | | | BD18 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD15 | | | | | | |
| 815916 | Library | P(T5) | BCD | IPTG/Lac | Bba_J61048 | | 2 | | 449 |
| | | 2xlacO | RBS_ | | | | | | |
| | | | alt1_ | | | | | | |
| | | | BD18 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD11 | | | | | | |
| 815917 | Library | P(T5) | BCD | IPTG/Lac | Bba_J61048 | | 2 | | 537 |
| | | 2xlacO | RBS_ | | | | | | |
| | | | alt1_ | | | | | | |
| | | | BD18 | | | | | | |
| | | Ptac | BCD | IPTG/Lac | BBa_B0015, | No | | 4 | |
| | | | RBS_ | | T7 | | | | |
| | | | alt1_ | | | | | | |
| | | | BD6 | | | | | | |
| 815918 | Library | P(T5) | BCD | IPTG/Lac | Bba_J61048 | | 2 | | 581 |
| | | 2xlacO | RBS_ | | | | | | |
| | | | alt1_ | | | | | | |
| | | | BD1 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD15 | | | | | | |
| 815967 | Library | Ptac | BCD | IPTG/Lac | | | 2 | | 383 |
| | | | RBS_ | | | | | | |
| | | | alt1_ | | | | | | |
| | | | BD1 | | | | | | |
| | | Ptac | BCD | IPTG/Lac | BBa_B0015, | Yes | | 4 | |
| | | | RBS_ | | T7 | | | | |
| | | | alt4_ | | | | | | |
| | | | BD1 | | | | | | |
| 815992 | Library | P(T5) | BCD | IPTG/Lac | Bba_J61048 | | 3 | | 180 |
| | | 2xlacO | RBS_ | | | | | | |
| | | | alt1_ | | | | | | |
| | | | BD18 | | | | | | |
| | | P(T7) | T7_RBS | IPTG/Lac | pRSF-duet | No | | 4 | |
| | | | | | Pre-T7 | | | | |
| | | | | | Terminator | | | | |
| | | | | | Spacer- | | | | |
| | | | | | Terminator, | | | | |
| | | | | | T7 | | | | |
| 815993 | Library | P(T5) | BCD | IPTG/Lac | Bba_J61048 | | 3 | | 90.3 |
| | | 2xlacO | RBS_ | | | | | | |
| | | | alt1_ | | | | | | |
| | | | BD18 | | | | | | |
| | | Ptac | BCD | IPTG/Lac | BBa_B0015, | No | | 5 | |
| | | | RBS_ | | T7 | | | | |
| | | | alt4_ | | | | | | |
| | | | BD2 | | | | | | |
| 815995 | Library | P(T5) | BCD | IPTG/Lac | Bba_J61048 | | 3 | | 447 |
| | | 2xlacO | RBS_ | | | | | | |
| | | | alt1_ | | | | | | |
| | | | BD18 | | | | | | |
| | | P(T5) | BCD | IPTG/Lac | BBa_B0015, | Yes | | 5 | |
| | | 2xlacO | RBS_ | | T7 | | | | |
| | | | alt4_ | | | | | | |
| | | | BD15 | | | | | | |
| 815996 | Library | P(T5) | BCD | IPTG/Lac | Bba_J61048 | | 3 | | 416 |
| | | 2xlacO | RBS_ | | | | | | |
| | | | alt1_ | | | | | | |
| | | | BD18 | | | | | | |
| | | Ptac | BCD | IPTG/Lac | BBa_B0015, | No | | 5 | |
| | | | RBS_ | | T7 | | | | |
| | | | alt4_ | | | | | | |
| | | | BD11 | | | | | | |
| 816008 | Library | P(T5) | BCD | IPTG/Lac | Bba_J61048 | | 3 | | 447 |
| | | 2xlacO | RBS_ | | | | | | |
| | | | alt1_ | | | | | | |
| | | | BD1 | | | | | | |
| | | Ptac | BCD | IPTG/Lac | BBa_B0015, | No | | 5 | |
| | | | RBS_ | | T7 | | | | |
| | | | alt4_ | | | | | | |
| | | | BD2 | | | | | | |
| 816044 | Library | P(T5) | BCD | IPTG/Lac | Bba_J61048 | | 2 | | 463 |
| | | 2xlacO | RBS_ | | | | | | |
| | | | alt1_ | | | | | | |
| | | | BD14 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD2 | | | | | | |
| 816045 | Library | P(T5) | BCD | IPTG/Lac | Bba_J61048 | | 2 | | 87.5 |
| | | 2xlacO | RBS_ | | | | | | |
| | | | alt1_ | | | | | | |
| | | | BD14 | | | | | | |
| | | P(T7) | T7_RBS | IPTG/Lac | BBa_B0015, | No | | 4 | |
| | | | | | T7 | | | | |
| 816046 | Library | P(T5) | BCD | IPTG/Lac | Bba_J61048 | | 2 | | 180 |
| | | 2xlacO | RBS_ | | | | | | |
| | | | alt1_ | | | | | | |
| | | | BD18 | | | | | | |
| | | P(T7) | T7_RBS | IPTG/Lac | BBa_B0015, | No | | 4 | |
| | | | | | T7 | | | | |
| 816055 | Library | P(T5) | BCD | IPTG/Lac | Bba_J61048 | | 2 | | 312 |
| | | 2xlacO | RBS_ | | | | | | |
| | | | alt1_ | | | | | | |
| | | | BD14 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD11 | | | | | | |
| 816056 | Library | P(T5) | BCD | IPTG/Lac | Bba_J61048 | | 2 | | 483 |
| | | 2xlacO | RBS_ | | | | | | |
| | | | alt1_ | | | | | | |
| | | | BD14 | | | | | | |
| | | Ptac | BCD | IPTG/Lac | BBa_B0015, | No | | 4 | |
| | | | RBS_ | | T7 | | | | |
| | | | alt1_ | | | | | | |
| | | | BD6 | | | | | | |
| 816057 | Library | P(T5) | BCD | IPTG/Lac | Bba_J61048 | | 2 | | 581 |
| | | 2xlacO | RBS_ | | | | | | |
| | | | alt1_ | | | | | | |
| | | | BD10 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD2 | | | | | | |
| 816070 | Library | P(T5) | BCD | IPTG/Lac | Bba_J61048 | | 2 | | 461 |
| | | 2xlacO | RBS_ | | | | | | |
| | | | alt1_ | | | | | | |
| | | | BD10 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD15 | | | | | | |
| 816071 | Library | P(T5) | BCD | IPTG/Lac | Bba_J61048 | | 2 | | 474 |
| | | 2xlacO | RBS_ | | | | | | |
| | | | alt1_ | | | | | | |
| | | | BD18 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD2 | | | | | | |
| 816072 | Library | P(T5) | BCD | IPTG/Lac | Bba_J61048 | | 2 | | 477 |
| | | 2xlacO | RBS_ | | | | | | |
| | | | alt1_ | | | | | | |
| | | | BD10 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD11 | | | | | | |
| 816073 | Library | P(T5) | BCD | IPTG/Lac | Bba_J61048 | | 2 | | 387 |
| | | 2xlacO | RBS_ | | | | | | |
| | | | alt1_ | | | | | | |
| | | | BD1 | | | | | | |
| | | Ptac | BCD | IPTG/Lac | BBa_B0015, | No | | 4 | |
| | | | RBS_ | | T7 | | | | |
| | | | alt4_ | | | | | | |
| | | | BD2 | | | | | | |

In the Ambr 250s fermentations, a protein drop was observed in some bioreactors toward the end of the time course. This may have been due to one or more of: cell lysis and decrease in optical density, protein degradation, protein insolubility when high concentrations were reached, and/or plasmid maintenance due to poor selection over the fermentation period.

VCE protein production between the two fermentation models (plate-based fermentation and Ambr 250s fermentation) was not found to correlate, so an additional metric of enrichment scoring (a comparison between the % in the total library vs. the % in the top hits) was used to evaluate the candidate VCE production library strains based on the plate-based fermentation assay described in Example 1. The library strains were subject to enrichment scoring of genetic parts (promoter, RBS, recoded VCE sequences, and terminators) used for the construction of the VCE-expressing plasmids in order to determine which combinations of genetic parts were more effective for VCE production than other combinations. Table 3 shows total numbers of VCE-producing library strains that showed enrichment for certain promoters. Table 4 shows total numbers of VCE-producing library strains that showed enrichment for certain RBSs for transcription and translation of the VCE D1 subunit.

TABLE 3
Enrichment Analysis of VCE Promoters

| | | Counts | Percentage | Counts | Percentage | % |
|---|---|---|---|---|---|---|
| Promoter | Inducer | (Library) | (Library) | (Top 30) | (Top 30) | Enrichment |
| P(T7) | IPTG/Lactose | 79 | 25.3 | 8 | 26.66 | 5.3 |
| P(T5) | IPTG/Lactose | 49 | 15.7 | 20 | 66.66 | 324.5 |
| Ptac | IPTG/Lactose | 16 | 5.1 | 1 | 3.33 | −34.7 |
| P(Llac01) | IPTG/Lactose | 14 | 4.4 | 0 | 0 | −100 |
| Various | n/a | 18 | 5.7 | 0 | 0 | −100 |
| Various | Vanillic Acid | 39 | 12.5 | 0 | 0 | −100 |
| Various | Cuminic Acid | 46 | 14.7 | 1 | 3 | −79.5 |
| Various | Anhydrotetracycline | 51 | 16.3 | 0 | 0 | −100 |
| TOTAL | | 312 | 99.7 | 30 | 100 | |

TABLE 4
Enrichment Analysis of VCE Subunit D1 RBSs

| | Counts | Percentage | Counts | Percentage | % |
|---|---|---|---|---|---|
| D1 RBS | (Library) | (Library) | (Top 41) | (Top 41) | Enrichment |
| BCDRBS_alt1_BD1 | 22 | 12 | 13 | 31.7 | 164 |
| BCDRBS_alt4_BD2 | 13 | 7 | 0 | 0 | −100 |
| BCDRBS_alt1_BD5 | 11 | 5.8 | 0 | 0 | −100 |
| BCDRBS_alt1_BD8 | 7 | 3.7 | 0 | 0 | −100 |
| BCDRBS_alt1_BD10 | 16 | 8.5 | 8 | 19.5 | 129 |
| BCDRBS_alt1_BD14 | 24 | 13 | 9 | 22 | 69 |
| BCDRBS_alt1_BD18 | 18 | 9.5 | 10 | 24 | 152 |
| T7-RBS | 77 | 41 | 1 | 2.4 | −94 |
| TOTAL | 188 | 100 | 41 | 100 | |

Based on the enrichment of genetic parts among the ˜300 library strains tested in the plate-based fermentation model (Table 3 and Table 4) and the VCE protein production performance of the 23 strains tested in Ambr 250s fermentation model (FIG. 2), 8 candidate VCE production library strains, corresponding to strain IDs 816008, 816072, 816070, 816056, 807172, 807173, 815995, and 815917, were selected and re-screened for VCE production using the Ambr 250s fermentation method described above. Despite the Ptac promoter exhibiting negative enrichment in Table 3, strain 807173, which comprised the Ptac promoter, was one of the strains selected because it was found in the Ambr 250s fermentation assay to produce comparable VCE titers relative to other strains but with less accumulated biomass (i.e., higher specific VCE titer per gram of cell pellet).

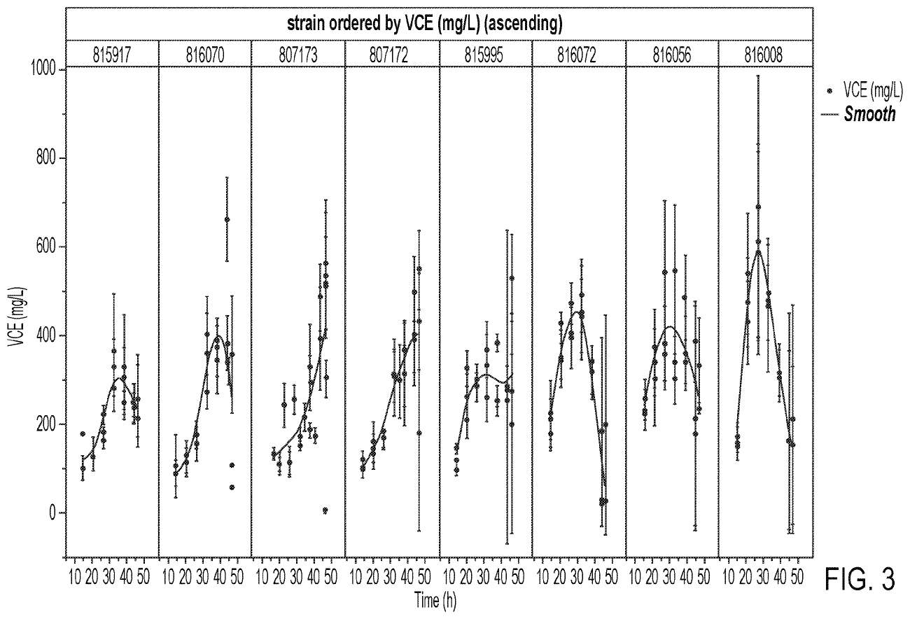

Soluble enzyme titers of VCE (mg/L) for each strain were measured from a 50 hour fed batch fermentation at the following time points: 15 hours, 20 hours, 26 hours, 32 hours, 38 hours, 44 hours, and 46 hours. The time course data was taken from 3 bioreactor replicates. Error bars show analytical variance across 4 lysis replicates (FIG. 3).

Thus, out of the ˜300 library strains tested, specific combinations of genetic components were identified that were effective for VCE production. Without wishing to be bound by any theory, the recoded nucleic acids encoding D1 and/or D12 provided in this disclosure, expressed under the control of specific combinations of synthetic promoters, RBSs, and/or terminators described in this disclosure, may provide an improved balance of D1:D12 co-expression, including sufficient expression of D12, which may lead to improved stabilization of the D1 subunit, resulting in increased yields of VCE.

### Example 3: Effect of Inducer on VCE Titer in E. coli VCE-Production Strains

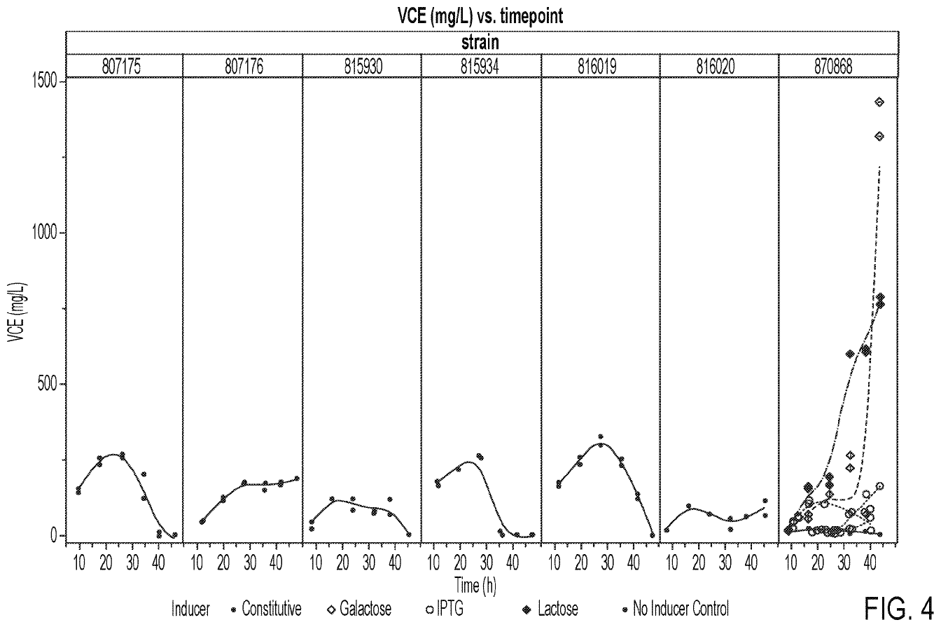

6 candidate VCE production library strains (strains 807175, 807176, 815930, 815934, 816019, and 816020), harboring constitutive VCE expression plasmids, were evaluated in comparison to a VCE production library strain (strain 870868) harboring an inducible VCE expression plasmid for VCE production using the Ambr 250s fermentation method. A variety of inducers were tested for strain 870868 (IPTG, lactose, galactose, and no inducer). For the constitutive VCE expression strains, no inducer was added. Soluble enzyme titers of VCE (mg/L) for each strain were measured from a 50 hour fed batch fermentation at the following time points: 10 hours, 18 hours, 26 hours, 35 hours, 41 hours, and 46 hours. The time course data were taken from 2 bioreactor replicates (FIG. 4). Lactose and galactose were observed to be more effective inducers of VCE production than IPTG.

TABLE 5
VCE Strain Data in Ambr 250s Fermentation System

| | | | | | | Tran- | | |
|---|---|---|---|---|---|---|---|---|
| | | | | | | script | SEQ ID | SEQ ID |
| | | | | | | shared | NO of | NO of |
| | | | | | | with | D1R | D12L |
| Strain | Strain | | | | | VCE- | nucleic | nucleic |
| ID | Type | Promoter | RBS | Inducer | Terminator | D12L | acid | acid |
| 870868 | Library | P(T5) | BCDRBS_ | IPTG/Lac/ | Bba_J61048 | | 2 | |
| | | 2xlacO | alt1_ | Gal | | | | |
| | | | BD1 | | | | | |
| | | P(Tac) | BCDRBS_ | IPTG/Lac/ | BBa_B0015, T7 | No | | 4 |
| | | | alt1_ | Gal | | | | |
| | | | BD6 | | | | | |
| 807175 | Library | apFAB124 | BCDRBS_ | None | None | | 3 | |
| | | | alt1_ | | | | | |
| | | | BD14 | | | | | |
| | | | BCDRB | None | BBa_B0015 | Yes | | 5 |
| | | | S_alt1_ | | | | | |
| | | | BD15 | | | | | |
| 807176 | Library | apFAB69 | BCDRBS_ | None | None | | 3 | |
| | | | alt1_ | | | | | |
| | | | BD14 | | | | | |
| | | | BCDRBS_ | None | BBa_B0015 | Yes | | 5 |
| | | | alt1_ | | | | | |
| | | | BD21 | | | | | |
| 815930 | Library | apFAB124 | BCDRBS_ | None | None | | 2 | |
| | | | alt1_ | | | | | |
| | | | BD14 | | | | | |
| | | | BCDRBS_ | None | BBa_B0015 | Yes | | 4 |
| | | | alt1_ | | | | | |
| | | | BD21 | | | | | |
| 815934 | Library | apFAB124 | BCDRBS_ | None | None | | 2 | |
| | | | alt1_ | | | | | |
| | | | BD14 | | | | | |
| | | | BCDRBS_ | None | BBa_B0015 | Yes | | 4 |
| | | | alt1_ | | | | | |
| | | | BD15 | | | | | |
| 816019 | Library | apFAB277 | BCDRBS_ | None | None | | 3 | |
| | | | alt1_ | | | | | |
| | | | BD14 | | | | | |
| | | | BCDRBS_ | None | BBa_B0015 | Yes | | 5 |
| | | | alt1_ | | | | | |
| | | | BD15 | | | | | |
| 816020 | Library | apFAB277 | BCDRBS_ | None | None | | 3 | |
| | | | alt1_ | | | | | |
| | | | BD14 | | | | | |
| | | | BCDRBS_ | None | BBa_B0015 | Yes | | 5 |
| | | | alt1_ | | | | | |
| | | | BD21 | | | | | |

### Example 4: Overexpression of ftsZ to Decrease Cell Elongation

Increased VCE production in cells may lead to an increase in viscosity and a slowing of fermentation. Without wishing to be bound by any theory, the increase in viscosity may be due to cell elongation caused by over-expression of VCE. To reduce the risk of increased viscosity due to cell elongation in VCE production host cells, expression of the ftsZ gene may be increased in the candidate VCE production library strains from Example 2. For example, one or more plasmids expressing one or more copies of the ftsZ gene may be expressed in the VCE production library strains and/or one or more copies of the ftsZ gene may be integrated into the genome of the VCE production library strains.

VCE production library strains that have increased expression of the ftsZ gene are screened using an Ambr 250s fermentation assay as described in Example 2, and total VCE concentration (mg/L) is determined. Cellular elongation and viscosity are also measured (e.g., by microscopic visualization and by a viscometer, respectively) and compared with the corresponding VCE production library strains that do not have increased expression of the ftsZ gene.

### Example 5: Supplementation with SAM- and GTP-Related Metabolites to Decrease Cell Elongation

To reduce the risk of increased viscosity due to cell elongation in VCE production host cells, candidate VCE production library strains from Example 2 are grown in fermentation broth that is supplemented with SAM- and GTP-related metabolites. VCE production library strains cultured in the presence of SAM- and GTP-related metabolites are screened using an Ambr 250s fermentation assay as described in Example 2, and total VCE concentration (mg/L) is determined. The cultures are either supplemented with a one-time injection or continuously supplemented with SAM- and GTP-related metabolites to increase the activity of native FtsZ. Cellular elongation and viscosity are also measured (e.g., by microscopic visualization and by a viscometer, respectively) and compared between the VCE production library strains cultured in the presence of SAM- and GTP-related metabolites and the corresponding VCE production library strains that are not cultured in the presence of SAM- and GTP-related metabolites.

### Example 6: Overexpression of metK and/or mreB to Regulate Cell Size and/or Morphology

VCE overexpression may influence the expression of genes such as metK, which encodes a SAM synthetase, and mreB, which may lead to an impact on cell growth and/or morphology. In order to alleviate any impact on cell growth and/or morphology, expression of the metK and/or mreB genes may be increased in the candidate VCE production library strains from Example 2. For example, one or more plasmids expressing one or more copies of the metK and/or mreB genes may be expressed in the VCE production library strains and/or one or more copies of the metK and/or mreB genes may be integrated into the genome of the VCE production library strains.

VCE production library strains that have increased expression of the metK and/or mreB genes are screened using an Ambr 250s fermentation assay as described in Example 2, and total VCE concentration (mg/L) is determined. Cellular elongation and viscosity are also measured (e.g., by microscopic visualization and by a viscometer, respectively) and compared with the corresponding VCE production library strains that do not have increased expression of the metK and/or mreB genes.

TABLE 6
Sequences Associated with the Disclosure

| SEQ | | |
|---|---|---|
| ID | Sequence | |
| NO: | Information | Sequence |
| 1 | P(T7) | taatacgactcactatag |
| | promoter | |
| 2 | D1 E. coli | atgaaacatcaccatcaccatcaccccatgagcgattacgacatccccactactgagaatctttattttcagggcgccgacgcta |
| | recode 1 | atgtcgtgtcttcttctaccatcgcaacctatattgacgctctggcaaaaaacgcctcggaactggaacaacgctcaaccgcgta |
| | (including His | tgaaatcaacaatgaactggaactggtgtttatcaaaccgccgctgattacgctgaccaacgtggttaatatcagcaccattcag |
| | tag) | gaatcttttattcgtttcacggttaccaacaaagaaggcgtcaaaatccgcacgaaaattccgctgagcaaagttcatggtctgg |
| | | atgtgaaaaacgttcaactggtcgacgcaatcgataatattgtgtgggaaaagaaaagcctggttaccgaaaatcgtctgcata |
| | | aagaatgcctgctgcgtctgagcacggaagaacgccacatctttctggactataaaaaatacggcagctctatccgcctggaa |
| | | ctggtgaacctgatccaggctaaaaccaaaaacttcacgatcgatttcaaactgaaatattttctgggcagtggtgctcaatcca |
| | | aaagttccctgctgcatgcgatcaaccacccgaaaagtcgtccgaatacctccctggaaattgaattcacccegcgcgacaac |
| | | gaaacggtgccgtacgatgaactgattaaagaactgaccacgctgtcacgtcatatctttatggcgtcgccggaaaacgttatt |
| | | ctgagcccgccgatcaatgccccgattaaaaccttcatgctgccgaaacaggacattgttggcctggatctggaaaacctgtat |
| | | gcggtcacgaaaaccgatggtattccgatcaccattcgcgtgacgtcgaatggcctgtattgctactttacccacctgggttatat |
| | | tatccgttacccggttaaacgcattatcgactccgaagtcgtggttttcggcgaagcggtcaaagataaaaattggaccgtgtat |
| | | ctgatcaaactgattgaaccggtgaacgccatcaacgatcgtctggaagaatcaaaatacgtggaatcgaaactggttgacat |
| | | ctgtgatcgcatcgttttcaaaagcaaaaaatacgaaggtccgttcaccacgacctctgaagtcgtggatatgctgagtacctat |
| | | ctgccgaaacagccggaaggcgtgatcctgttttacagcaaaggtccgaaatctaacatcgacttcaaaatcaaaaaagaaaa |
| | | caccatcgatcaaacggccaatgttgtctttcgttatatgtcatcggaaccgattatctttggcgaaagctctatcttcgtggaata |
| | | caaaaaattctcgaacgataaaggcttcccgaaagaatacggcagcggtaaaattgtcctgtataacggtgtgaattacctgaa |
| | | caatatctattgcctggaatacattaacacccataatgaagttggcattaaatctgtggttgtcccgatcaaatttattgcagaattc |
| | | ctggtcaacggtgaaatcctgaaaccgcgtattgacaaaaccatgaaatacatcaacagtgaagattactacggtaaccagca |
| | | taacatcatcgtggaacacctgcgcgaccaatctatcaaaatcggcgatatcttcaacgaagacaaactgagtgatgtcggtca |
| | | ccagtatgcgaacaatgataaatttcgtctgaacccggaagtgtcctacttcaccaataaacgtacgcgcggcccgctgggtat |
| | | cctgtcaaattatgtcaaaaccctgctgatttcaatgtactgttcgaaaacgtttctggatgacagcaacaaacgcaaagttctgg |
| | | ccattgactttggcaatggtgcagatctggaaaaatatttctacggcgaaatcgctctgctggttgcgaccgatccggacgcgg |
| | | atgccattgcacgtggcaacgaacgctataacaaactgaattctggtatcaaaaccaaatactacaaattcgactacatccagg |
| | | aaaccattcgtagtgatacgttcgtgagttccgttcgcgaagtcttttatttcggcaaattcaacatcatcgattggcaattcgccat |
| | | ccattattctttccatccgcgtcactacgcaaccgtgatgaacaatctgagtgaactgacggcttccggcggtaaagttctgatta |
| | | cgacgatggatggtgataaactgtccaaactgaccgataagaaaaccttcattatccacaaaaacctgccgtcatcggaaaact |
| | | acatgtcagtggaaaaaatcgccgatgaccgcattgtggtttataacccgagcacgatgtctaccccgatgacggaatacatc |
| | | attaagaaaaacgatatcgtccgtgtgtttaatgaatacggtttcgttctggtcgacaacgttgattttgcaaccattatcgaacgc |
| | | agcaaaaaattcatcaatggcgcttccacgatggaagatcgtccgtcaacgcgcaactttttcgaactgaatcgcggtgcaatt |
| | | aaatgtgaaggtctggatgtggaagatctgctgtcctattatgtcgtgtatgtgttctctaaacgctaa |
| 3 | D1 E. coli | atgaaacatcaccatcaccatcaccccatgagegattacgacatccccactactgagaatctttattttcagggcgccgacgcc |
| | recode 12 | aacgtagtgagctcgtccacgattgctacatacatcgacgcactggctaaaaacgcgagtgaattagagcaacgttcaaccgc |
| | (including His | ctatgaaatcaacaacgaacttgagctcgtctttattaagcctccgctaatcaccctgactaacgttgttaatatatctaccatcca |
| | tag) | ggaaagcttcattcgcttcactgttactaacaaagaaggcgtaaaaatcaggactaaaatcccattgtctaaggtgcacgggct |
| | | ggatgtgaaaaacgttcagctggttgacgctattgacaacatcgtatgggaaaagaaatccctcgtaaccgaaaaccgtctgc |
| | | ataaagaatgtctgctgcgtctgagcacggaggaacgacacatctttctggattacaaaaaatatggtagttctattcgtctgga |
| | | gctggtgaacctgatccaggcaaagaccaaaaatttcacaattgacttcaaactaaaatactttctgggctccggtgcgcagag |
| | | caaatcttccctgttgcatgctatcaaccacccgaaaagccgcccgaatacttctctggaaatcgagttcaccccccgcgataa |
| | | cgaaactgtcccatacgatgagcttattaaggaactgaccacgctgtcccgtcacatttttatggcgagcccggaaaacgttata |
| | | ttatogccgcctatcaacgctccgatcaagaccttcatgttgccgaaacaagacatcgtcggtctggatctggagaacctgtac |
| | | gcagttactaaaaccgacggcatccccatcactatcagagtaacgtcaaacggattgtattgctatttcacccatctgggttacat |
| | | tattcgttacccggtgaaacgcatcatagattctgaagttgttgttttcggcgaagccgtaaaggacaaaaactggaccgtctatc |
| | | tgatcaagctaatcgaaccggttaatgctatcaacgatcggctggaagaatcgaaatacgtagaatctaaactggtggatatttg |
| | | cgaccgtattgtctttaaatcgaaaaagtacgagggtcctttcactactactagcgaagtcgtggacatgctctctacgtacctgc |
| | | cgaaacagcctgagggcgttatcctgttctatagcaaaggtccgaaatccaacatcgattttaagattaaaaaggaaaacacca |
| | | ttgatcagacggctaatgtagttttccggtacatgtctagcgagccgatcatctttggcgaatcttctatctttgtagaatataaaaa |
| | | gttcagcaacgacaaaggattcccaaaagaatacgggtccgggaaaatcgtcttatacaacggtgttaactacttgaacaacat |
| | | ctattgcctggaatatatcaatactcacaatgaagttggtattaaatcagtggttgttccgataaaattcatcgcggaatttctggtc |
| | | aatggcgaaatcctgaaaccccgcattgataagaccatgaaatacataaactccgaagactactacggtaaccagcataacat |
| | | catcgtggaacacctgagagatcagagtatcaaaatcggcgacattttcaatgaggacaagttaagcgacgtgggccatcaat |
| | | acgcaaacaacgacaaattccgtctgaacccggaggtttcctatttcaccaacaaacgtacccgaggtccgcttggcatcctct |
| | | ccaattacgtaaaaaccctgctgatttctatgtattgttcaaaaacgttcctggatgacagcaacaaaaggaaggtactggctatc |
| | | gatttcggtaacggcgcggatctggaaaagtacttttacggtgaaatcgctctgttagtcgcaactgatccggacgccgacgca |
| | | attgctcgcggaaatgaacgttacaacaaactgaactccggtattaaaacaaagtattataaattcgactatatccaggagactat |
| | | ccgctctgatactttcgtgagcagcgtgcgtgaggttttttactttggtaaattcaacattattgactggcagtttgcgatccactac |
| | | agctttcacccgcgtcactatgcgaccgttatgaataacctatcggaactcacggctagcggcggcaaagtgctgattactact |
| | | atggacggtgacaaactgtctaagctgaccgataagaaaaccttcatcatccacaaaaacttgccaagttctgagaactatatgt |
| | | ctgttgaaaaaattgcggacgaccgcatcgtcgtttacaacccatctaccatgtccacccctatgacagagtacatcatcaaaaa |
| | | gaacgacatagttcgtgttttcaacgaatacggcttcgtactggtagataacgtcgattttgctaccattatcgagcgttcgaaaa |
| | | aattcattaacggtgcttccactatggaagatcgtccgtccactcgtaacttttttgaattaaaccgtggcgcaatcaaatgcgaa |
| | | gggctggatgtggaagacctcctgtcttactacgttgtatacgtcttctctaaacgctaa |
| 4 | D12 E. coli | atggatgaaatcgtcaaaaatatccgcgaaggcacgcacgtcctgctgccgttctatgaaaccctgccggaactgaatctgtc |
| | recode 1 | actgggcaaatctccgctgccgagtctggaatatggtgcaaactactttctgcagatttctcgtgtgaacgatctgaatcgcatg |
| | (including | ccgaccgacatgctgaaactgttcacgcatgatatcatgctgccggaaagcgatctggacaaagtctacgaaatcctgaaaat |
| | Twin Strep- | caactccgttaaatactacggccgttcaaccaaagcggatgccgtggttgcagacctgtccgctcgcaataaactgtttaaacg |
| | tag | tgaacgcgatgctattaaatcgaacaatcacctgaccgaaaacaacctgtacatcagcgattacaaaatgctgacgtttgacgt |
| | | gttccgtccgctgttcgatttcgttaacgaaaaatactgcatcatcaaactgccgaccctgtttggccgtggtgtgattgatacgat |
| | | gcgcatctactgcagcctgttcaaaaatgtccgcctgctgaaatgtgtgtcggatagctggctgaaagactctgcgattatggtg |
| | | gccagtgacgtttgtaagaaaaacctggacctgtttatgtcccatgtcaaatcagtgaccaaaagctctagttggaaagacgtta |
| | | attcggtccaatttagcattctgaacaatccggttgatacggaattcatcaacaaattcctggaattctctaaccgtgtttacgaag |
| | | cactgtattacgtccacagtctgctgtactcctcaatgaccteggactccaaatccatcgaaaataaacatcaacgccgcctggt |
| | | gaaactgctgctggggagcgcttggagccacccgcagttcgaaaaaggtggaggttctggcggtggatcgggaggttcagc |
| | | gtggagccacccgcagttcgagaaataa |
| 5 | D12 E. coli | atggatgagatcgttaagaacattcgtgaaggtacgcatgtgcttttgccattttacgaaactctcccggaactgaatctgtcctta |
| | recode 2 | ggcaaaagccctctaccctctctggagtatggggccaactacttcctgcaaatctcacgcgtcaacgacctgaatcgaatgcc |
| | (including | gaccgacatgctgaaactgttcactcacgatataatgctgccggaaagtgatctggacaaagtatatgaaatcctgaaaatcaa |
| | Twin Strep- | cagcgttaagtactacggacggtcgaccaaagcggacgctgttgtagcagatctgtctgctcgcaacaaactctttaaacgtga |
| | tag) | acgtgacgctattaagtccaacaaccacctgacagagaacaatctctatatctctgactacaaaatgttgactttcgatgtgttcc |
| | | gtccgctgtttgatttcgtgaacgaaaaatattgcattatcaaactgccgaccctgttcggccgtggtgttattgacaccatgcgc |
| | | atctactgtagcctcttcaagaatgtcagactactgaaatgcgtgtccgatagctggctgaaagacagcgcaatcatggtagcc |
| | | tcagacgtttgcaaaaagaacctggatctgtttatgtcccatgttaaatccgttactaagtctagctcgtggaaagatgttaacagc |
| | | gtacagttttctattttgaacaaccctgttgacacggaatttatcaacaaattcctggagttctctaaccgtgtatacgaagcgctgt |
| | | attacgtgcactccttactgtactcttctatgaccagcgatagtaagtctatcgaaaataaacaccagcgccgtctggtaaaactg |
| | | ctccttgggagcgcttggagccacccgcagttcgaaaaaggtggaggttctggcggtggatcgggaggttcagcgtggagc |
| | | cacccgcagttcgagaaataa |
| 6 | D1 amino acid | MKHHHHHHPMSDYDIPTTENLYFQGADANVVSSSTIATYIDALAKNASELEQ |
| | sequence | RSTAYEINNELELVFIKPPLITLTNVVNISTIQESFIRFTVTNKEGVKIRTKIPLSKV |
| | (including His- | HGLDVKNVQLVDAIDNIVWEKKSLVTENRLHKECLLRLSTEERHIFLDYKKYG |
| | tag in bold) | SSIRLELVNLIQAKTKNFTIDFKLKYFLGSGAQSKSSLLHAINHPKSRPNTSLEIEF |
| | | TPRDNETVPYDELIKELTTLSRHIFMASPENVILSPPINAPIKTFMLPKQDIVGLDL |
| | | ENLYAVTKTDGIPITIRVTSNGLYCYFTHLGYIIRYPVKRIIDSEVVVFGEAVKDK |
| | | NWTVYLIKLIEPVNAINDRLEESKYVESKLVDICDRIVFKSKKYEGPFTTTSEVV |
| | | DMLSTYLPKQPEGVILFYSKGPKSNIDFKIKKENTIDQTANVVFRYMSSEPIIFGE |
| | | SSIFVEYKKFSNDKGFPKEYGSGKIVLYNGVNYLNNIYCLEYINTHNEVGIKSVV |
| | | VPIKFIAEFLVNGEILKPRIDKTMKYINSEDYYGNQHNIIVEHLRDQSIKIGDIFNE |
| | | DKLSDVGHQYANNDKFRLNPEVSYFTNKRTRGPLGILSNYVKTLLISMYCSKTF |
| | | LDDSNKRKVLAIDFGNGADLEKYFYGEIALLVATDPDADAIARGNERYNKLNS |
| | | GIKTKYYKFDYIQETIRSDTFVSSVREVFYFGKFNIIDWQFAIHYSFHPRHYATV |
| | | MNNLSELTASGGKVLITTMDGDKLSKLTDKKTFIIHKNLPSSENYMSVEKIADD |
| | | RIVVYNPSTMSTPMTEYIIKKNDIVRVFNEYGFVLVDNVDFATIIERSKKFINGAS |
| | | TMEDRPSTRNFFELNRGAIKCEGLDVEDLLSYYVVYVFSKR |
| 7 | D12 amino | MDEIVKNIREGTHVLLPFYETLPELNLSLGKSPLPSLEYGANYFLQISRVNDLNR |
| | acid sequence | MPTDMLKLFTHDIMLPESDLDKVYEILKINSVKYYGRSTKADAVVADLSARNK |
| | (including | LFKRERDAIKSNNHLTENNLYISDYKMLTFDVFRPLFDFVNEKYCIIKLPTLFGR |
| | Twin Strep- | GVIDTMRIYCSLFKNVRLLKCVSDSWLKDSAIMVASDVCKKNLDLFMSHVKSV |
| | tag in bold) | TKSSSWKDVNSVQFSILNNPVDTEFINKFLEFSNRVYEALYYVHSLLYSSMTSD |
| | | SKSIENKHQRRLVKLLLGSAWSHPQFEKGGGSGGGSGGSAWSHPQFEK |
| 8 | Ptac promoter | tgttgacaattaatcatcggctcgtataatgtgtggaattgtgagcgctcacaatt |
| 9 | P(T5) 2xlacO | aattgtgagcggataacaattacgagcttcatgcacagtgaaatcatgaaaaatttatttgctttgtgagcggataacaattataat |
| | promoter | atgtggaattgtgagcgctcacaattccaca |
| 10 | BCDRBS_alt1_ | gcgaaaaatcaataaggaggcaacaagatgtgcgaaaaacatcttaatcatgcacaggagactttcta |
| | BD1 | |
| 11 | BCDRBS_alt4_ | gtcaataaaggcatataaaaggaggttaataacatgaaagttaaagtaaaacatcttaatcatgctaaggaggttttcta |
| | BD2 RBS | |
| 12 | BCDRBS_alt1_ | gcgaaaaatcaataaggaggcaacaagatgtgcgaaaaacatcttaatcatgcgccggaggttttcta |
| | BD6 | |
| 13 | BCDRBS_alt1_ | gcgaaaaatcaataaggaggcaacaagatgtgcgaaaaacatcttaatcatgcggaggatcgtttcta |
| | BD10 | |
| 14 | BCDRBS_alt4_ | gtcaataaaggcatataaaaggaggttaataacatgaaagttaaagtaaaacatcttaatcatgcgggggagtgtttcta |
| | BD11 | |
| 15 | BCDRBS_alt1_ | gcgaaaaatcaataaggaggcaacaagatgtgcgaaaaacatcttaatcatgcggtggagggtttcta |
| | BD14 | |
| 16 | BCDRBS_alt4_ | gtcaataaaggcatataaaaggaggttaataacatgaaagttaaagtaaaacatcttaatcatgcgggggagtctttcta |
| | BD15 | |
| 17 | BCDRBS_alt1_ | gcgaaaaatcaataaggaggcaacaagatgtgcgaaaaacatcttaatcatgcgacggagcgtttcta |
| | BD18 | |
| 18 | Bba_J61048 | ccggcttatcggtcagtttcacctgatttacgtaaaaacccgcttcggcgggtttttgcttttggaggggcagaaagatgaatga |
| | Terminator | ctgtccacgacgctatacccaaaagaaa |
| 19 | BBa_B0015 | ccaggcatcaaataaaacgaaaggctcagtcgaaagactgggcctttcgttttatctgttgtttgtcggtgaacgctctctactag |
| | Terminator | agtcacactggctcaccttcgggtgggcctttctgcgtttata |
| 20 | T7 Terminator | ataaccccttggggcctctaaacgggtcttgaggggttttttgc |
| 21 | Combination | aattgtgagcggataacaattacgagcttcatgcacagtgaaatcatgaaaaatttatttgctttgtgagcggataacaattataat |
| | of genetic | atgtggaattgtgagcgctcacaattccacagcgaaaaatcaataaggaggcaacaagatgtgcgaaaaacatcttaatcatg |
| | elements | cacaggagactttctaatgaaacatcaccatcaccatcaccccatgagegattacgacatccccactactgagaatctttattttc |
| | expressed in | agggcgccgacgctaatgtcgtgtcttcttctaccatcgcaacctatattgacgctctggcaaaaaacgcctcggaactggaac |
| | strain | aacgctcaaccgcgtatgaaatcaacaatgaactggaactggtgtttatcaaaccgccgctgattacgctgaccaacgtggtta |
| | 807172(Promo | atatcagcaccattcaggaatcttttattcgtttcacggttaccaacaaagaaggcgtcaaaatccgcacgaaaattccgctgag |
| | ter (P(T5) | caaagttcatggtctggatgtgaaaaacgttcaactggtcgacgcaatcgataatattgtgtgggaaaagaaaagcctggttac |
| | 2xlacO); RBS | cgaaaatcgtctgcataaagaatgcctgctgcgtctgagcacggaagaacgccacatctttctggactataaaaaatacggca |
| | (BCDRBS_alt1_ | gctctatccgcctggaactggtgaacctgatccaggctaaaaccaaaaacttcacgatcgatttcaaactgaaatattttctggg |
| | BD1); His- | cagtggtgctcaatccaaaagttccctgctgcatgcgatcaaccacccgaaaagtegtccgaatacctccctggaaattgaatt |
| | Tag; D1 (E. | caccccgcgcgacaacgaaacggtgccgtacgatgaactgattaaagaactgaccacgctgtcacgtcatatctttatggcgt |
| | coli recode 1); | cgccggaaaacgttattctgagcccgccgatcaatgccccgattaaaaccttcatgctgccgaaacaggacattgttggcctg |
| | Terminator | gatctggaaaacctgtatgcggtcacgaaaaccgatggtattccgatcaccattcgcgtgacgtcgaatggcctgtattgctact |
| | (Bba_J61048); | ttacccacctgggttatattatccgttacccggttaaacgcattatcgactccgaagtcgtggttttcggcgaagcggtcaaagat |
| | Promoter | aaaaattggaccgtgtatctgatcaaactgattgaaccggtgaacgccatcaacgatcgtctggaagaatcaaaatacgtgga |
| | (Ptac); RBS | atcgaaactggttgacatctgtgatcgcatcgttttcaaaagcaaaaaatacgaaggtccgttcaccacgacctctgaagtcgtg |
| | (BCDRBS_alt1_ | gatatgctgagtacctatctgccgaaacagccggaaggcgtgatcctgttttacagcaaaggtccgaaatctaacatcgacttc |
| | BD6); D12 | aaaatcaaaaaagaaaacaccatcgatcaaacggccaatgttgtctttcgttatatgtcatcggaaccgattatctttggcgaaa |
| | (E. coli recode | gctctatcttcgtggaatacaaaaaattctcgaacgataaaggcttcccgaaagaatacggcagcggtaaaattgtcctgtataa |
| | 1); Twin | cggtgtgaattacctgaacaatatctattgcctggaatacattaacacccataatgaagttggcattaaatctgtggttgtcccgat |
| | Strep Tag; | caaatttattgcagaattcctggtcaacggtgaaatcctgaaaccgcgtattgacaaaaccatgaaatacatcaacagtgaagat |
| | Terminator | tactacggtaaccagcataacatcatcgtggaacacctgcgcgaccaatctatcaaaatcggcgatatcttcaacgaagacaa |
| | ((BBa_B0015 | actgagtgatgtcggtcaccagtatgcgaacaatgataaatttcgtctgaacccggaagtgtcctacttcaccaataaacgtacg |
| | (Double | cgcggcccgctgggtatcctgtcaaattatgtcaaaaccctgctgatttcaatgtactgttcgaaaacgtttctggatgacagcaa |
| | Terminator | caaacgcaaagttctggccattgactttggcaatggtgcagatctggaaaaatatttctacggcgaaatcgctctgctggttgcg |
| | B0010, | accgatccggacgcggatgccattgcacgtggcaacgaacgctataacaaactgaattctggtatcaaaaccaaatactacaa |
| | B0012)); | attcgactacatccaggaaaccattcgtagtgatacgttcgtgagttccgttcgcgaagtcttttatttcggcaaattcaacatcatc |
| | Terminator | gattggcaattcgccatccattattctttccatccgcgtcactacgcaaccgtgatgaacaatctgagtgaactgacggcttccg |
| | (T7 | gcggtaaagttctgattacgacgatggatggtgataaactgtccaaactgaccgataagaaaaccttcattatccacaaaaacct |
| | terminator) | gccgtcatcggaaaactacatgtcagtggaaaaaatcgccgatgaccgcattgtggtttataacccgagcacgatgtctaccc |
| | | cgatgacggaatacatcattaagaaaaacgatatcgtccgtgtgtttaatgaatacggtttcgttctggtcgacaacgttgattttg |
| | | caaccattatcgaacgcagcaaaaaattcatcaatggcgcttccacgatggaagatcgtccgtcaacgcgcaactttttcgaac |
| | | tgaatcgcggtgcaattaaatgtgaaggtctggatgtggaagatctgctgtcctattatgtcgtgtatgtgttctctaaacgctaac |
| | | cggcttatcggtcagtttcacctgatttacgtaaaaacccgcttcggcgggtttttgcttttggaggggcagaaagatgaatgact |
| | | gtccacgacgctatacccaaaagaaatgttgacaattaatcatcggctcgtataatgtgtggaattgtgagcgctcacaattgcg |
| | | aaaaatcaataaggaggcaacaagatgtgcgaaaaacatcttaatcatgcgccggaggttttctaatggatgaaatcgtcaaaa |
| | | atatccgcgaaggcacgcacgtcctgctgccgttctatgaaaccctgccggaactgaatctgtcactgggcaaatctccgctg |
| | | ccgagtctggaatatggtgcaaactactttctgcagatttctcgtgtgaacgatctgaatcgcatgccgaccgacatgctgaaac |
| | | tgttcacgcatgatatcatgctgccggaaagcgatctggacaaagtctacgaaatcctgaaaatcaactccgttaaatactacg |
| | | gccgttcaaccaaagcggatgccgtggttgcagacctgtccgctcgcaataaactgtttaaacgtgaacgcgatgctattaaat |
| | | cgaacaatcacctgaccgaaaacaacctgtacatcagegattacaaaatgctgacgtttgacgtgttccgtccgctgttcgattt |
| | | cgttaacgaaaaatactgcatcatcaaactgccgaccctgtttggccgtggtgtgattgatacgatgcgcatctactgcagcctg |
| | | ttcaaaaatgtccgcctgctgaaatgtgtgtcggatagctggctgaaagactctgcgattatggtggccagtgacgtttgtaaga |
| | | aaaacctggacctgtttatgtcccatgtcaaatcagtgaccaaaagctctagttggaaagacgttaattcggtccaatttagcatt |
| | | ctgaacaatccggttgatacggaattcatcaacaaattcctggaattctctaaccgtgtttacgaagcactgtattacgtccacagt |
| | | ctgctgtactcctcaatgacctcggactccaaatccatcgaaaataaacatcaacgccgcctggtgaaactgctgctggggag |
| | | cgcttggagccacccgcagttcgaaaaaggtggaggttctggcggtggatcgggaggttcagcgtggagccacccgcagtt |
| | | cgagaaataaccaggcatcaaataaaacgaaaggctcagtcgaaagactgggcctttcgttttatctgttgtttgtcggtgaacg |
| | | ctctctactagagtcacactggctcaccttcggggggcctttctgcgtttataataaccccttggggcctctaaacgggtcttga |
| | | ggggttttttgc |
| 22 | Combination | tgttgacaattaatcatcggctcgtataatgtgtggaattgtgagcgctcacaattgcgaaaaatcaataaggaggcaacaagat |
| | of genetic | gtgcgaaaaacatcttaatcatgcggaggatcgtttctaatgaaacatcaccatcaccatcaccccatgagcgattacgacatc |
| | elements | cccactactgagaatctttattttcagggcgccgacgctaatgtcgtgtcttcttctaccatcgcaacctatattgacgctctggca |
| | expressed in | aaaaacgcctcggaactggaacaacgctcaaccgcgtatgaaatcaacaatgaactggaactggtgtttatcaaaccgccgct |
| | strain 807173 | gattacgctgaccaacgtggttaatatcagcaccattcaggaatcttttattcgtttcacggttaccaacaaagaaggcgtcaaaa |
| | (Promoter | tccgcacgaaaattccgctgagcaaagttcatggtctggatgtgaaaaacgttcaactggtcgacgcaatcgataatattgtgtg |
| | (Ptac); RBS | ggaaaagaaaagcctggttaccgaaaatcgtctgcataaagaatgcctgctgcgtctgagcacggaagaacgccacatctttc |
| | (BCDRBS_alt1_ | tggactataaaaaatacggcagctctatccgcctggaactggtgaacctgatccaggctaaaaccaaaaacttcacgatcgatt |
| | BD10); His- | tcaaactgaaatattttctgggcagtggtgctcaatccaaaagttccctgctgcatgcgatcaaccacccgaaaagtcgtccga |
| | Tag; D1 (E. | atacctccctggaaattgaattcaccccgcgcgacaacgaaacggtgccgtacgatgaactgattaaagaactgaccacgct |
| | coli recode 1); | gtcacgtcatatctttatggcgtcgccggaaaacgttattctgagcccgccgatcaatgccccgattaaaaccttcatgctgccg |
| | RBS | aaacaggacattgttggcctggatctggaaaacctgtatgcggtcacgaaaaccgatggtattccgatcaccattcgcgtgac |
| | (BCDRBS_alt4_ | gtcgaatggcctgtattgctactttacccacctgggttatattatccgttacccggttaaacgcattatcgactccgaagtcgtggtt |
| | BD11); D12 | ttcggcgaagcggtcaaagataaaaattggaccgtgtatctgatcaaactgattgaaccggtgaacgccatcaacgatcgtctg |
| | (E. coli recode | gaagaatcaaaatacgtggaatcgaaactggttgacatctgtgatcgcatcgttttcaaaagcaaaaaatacgaaggtccgttc |
| | 1); Twin Strep | accacgacctctgaagtcgtggatatgctgagtacctatctgccgaaacagccggaaggcgtgatcctgttttacagcaaagg |
| | Tag; | tccgaaatctaacatcgacttcaaaatcaaaaaagaaaacaccatcgatcaaacggccaatgttgtctttcgttatatgtcatcgg |
| | Terminator | aaccgattatctttggcgaaagctctatcttcgtggaatacaaaaaattctcgaacgataaaggcttcccgaaagaatacggca |
| | ((BBa_B0015 | gcggtaaaattgtcctgtataacggtgtgaattacctgaacaatatctattgcctggaatacattaacacccataatgaagttggc |
| | (Double | attaaatctgtggttgtcccgatcaaatttattgcagaattcctggtcaacggtgaaatcctgaaaccgcgtattgacaaaaccat |
| | Terminator | gaaatacatcaacagtgaagattactacggtaaccagcataacatcatcgtggaacacctgegcgaccaatctatcaaaatcg |
| | B0010, | gcgatatcttcaacgaagacaaactgagtgatgtcggtcaccagtatgcgaacaatgataaatttcgtctgaacccggaagtgt |
| | B0012)); | cctacttcaccaataaacgtacgcgcggcccgctgggtatcctgtcaaattatgtcaaaaccctgctgatttcaatgtactgttcg |
| | Terminator (T7 | aaaacgtttctggatgacagcaacaaacgcaaagttctggccattgactttggcaatggtgcagatctggaaaaatatttctacg |
| | terminator) | gcgaaatcgctctgctggttgcgaccgatccggacgcggatgccattgcacgtggcaacgaacgctataacaaactgaattct |
| | | ggtatcaaaaccaaatactacaaattcgactacatccaggaaaccattcgtagtgatacgttcgtgagttccgttcgcgaagtctt |
| | | ttatttcggcaaattcaacatcatcgattggcaattcgccatccattattctttccatccgcgtcactacgcaaccgtgatgaacaat |
| | | ctgagtgaactgacggcttccggcggtaaagttctgattacgacgatggatggtgataaactgtccaaactgaccgataagaa |
| | | aaccttcattatccacaaaaacctgccgtcatcggaaaactacatgtcagtggaaaaaatcgccgatgaccgcattgtggtttat |
| | | aacccgagcacgatgtctaccccgatgacggaatacatcattaagaaaaacgatatcgtccgtgtgtttaatgaatacggtttcg |
| | | ttctggtcgacaacgttgattttgcaaccattatcgaacgcagcaaaaaattcatcaatggcgcttccacgatggaagategtcc |
| | | gtcaacgcgcaactttttcgaactgaatcgcggtgcaattaaatgtgaaggtctggatgtggaagatctgctgtcctattatgtcg |
| | | tgtatgtgttctctaaacgctaagtcaataaaggcatataaaaggaggttaataacatgaaagttaaagtaaaacatcttaatcatg |
| | | cgggggagtgtttctaatggatgaaatcgtcaaaaatatccgcgaaggcacgcacgtcctgctgccgttctatgaaaccctgc |
| | | cggaactgaatctgtcactgggcaaatctccgctgccgagtctggaatatggtgcaaactactttctgcagatttctcgtgtgaa |
| | | cgatctgaatcgcatgccgaccgacatgctgaaactgttcacgcatgatatcatgctgccggaaagcgatctggacaaagtct |
| | | acgaaatcctgaaaatcaactccgttaaatactacggccgttcaaccaaagcggatgccgtggttgcagacctgtccgctcgc |
| | | aataaactgtttaaacgtgaacgcgatgctattaaatcgaacaatcacctgaccgaaaacaacctgtacatcagcgattacaaa |
| | | atgctgacgtttgacgtgttccgtccgctgttcgatttcgttaacgaaaaatactgcatcatcaaactgccgaccctgtttggccgt |
| | | ggtgtgattgatacgatgcgcatctactgcagcctgttcaaaaatgtccgcctgctgaaatgtgtgtcggatagctggctgaaag |
| | | actctgcgattatggtggccagtgacgtttgtaagaaaaacctggacctgtttatgtcccatgtcaaatcagtgaccaaaagctct |
| | | agttggaaagacgttaattcggtccaatttagcattctgaacaatccggttgatacggaattcatcaacaaattcctggaattctct |
| | | aaccgtgtttacgaagcactgtattacgtccacagtctgctgtactcctcaatgaccteggactccaaatccatcgaaaataaac |
| | | atcaacgccgcctggtgaaactgctgctggggagcgcttggagccacccgcagttcgaaaaaggtggaggttctggcggtg |
| | | gatcgggaggttcagcgtggagccacccgcagttcgagaaataaccaggcatcaaataaaacgaaaggctcagtcgaaag |
| | | actgggcctttcgttttatctgttgtttgtcggtgaacgctctctactagagtcacactggctcaccttcgggtgggcctttctgcgtt |
| | | tataataaccccttggggcctctaaacgggtcttgaggggttttttgc |
| 23 | Combination | aattgtgagcggataacaattacgagcttcatgcacagtgaaatcatgaaaaatttatttgctttgtgagcggataacaattataat |
| | of genetic | atgtggaattgtgagcgctcacaattccacagcgaaaaatcaataaggaggcaacaagatgtgcgaaaaacatcttaatcatg |
| | elements | cgacggagcgtttctaatgaaacatcaccatcaccatcaccccatgagegattacgacatccccactactgagaatctttattttc |
| | expressed in | agggcgccgacgctaatgtcgtgtcttcttctaccatcgcaacctatattgacgctctggcaaaaaacgcctcggaactggaac |
| | strain 815917 | aacgctcaaccgcgtatgaaatcaacaatgaactggaactggtgtttatcaaaccgccgctgattacgctgaccaacgtggtta |
| | (Promoter | atatcagcaccattcaggaatcttttattcgtttcacggttaccaacaaagaaggcgtcaaaatccgcacgaaaattccgctgag |
| | (P(T5) | caaagttcatggtctggatgtgaaaaacgttcaactggtcgacgcaatcgataatattgtgtgggaaaagaaaagcctggttac |
| | 2xlacO); RBS | cgaaaatcgtctgcataaagaatgcctgctgcgtctgagcacggaagaacgccacatctttctggactataaaaaatacggca |
| | (BCDRBS_alt1_ | gctctatccgcctggaactggtgaacctgatccaggctaaaaccaaaaacttcacgatcgatttcaaactgaaatattttctggg |
| | BD18); His- | cagtggtgctcaatccaaaagttccctgctgcatgcgatcaaccacccgaaaagtcgtccgaatacctccctggaaattgaatt |
| | Tag; D1 (E. | caccccgcgcgacaacgaaacggtgccgtacgatgaactgattaaagaactgaccacgctgtcacgtcatatctttatggcgt |
| | coli recode 1); | cgccggaaaacgttattctgagcccgccgatcaatgccccgattaaaaccttcatgctgccgaaacaggacattgttggcctg |
| | Terminator | gatctggaaaacctgtatgcggtcacgaaaaccgatggtattccgatcaccattcgcgtgacgtcgaatggcctgtattgctact |
| | (Bba_J61048); | ttacccacctgggttatattatccgttacccggttaaacgcattatcgactccgaagtcgtggttttcggcgaagcggtcaaagat |
| | Promoter | aaaaattggaccgtgtatctgatcaaactgattgaaccggtgaacgccatcaacgatcgtctggaagaatcaaaatacgtgga |
| | (Ptac); RBS | atcgaaactggttgacatctgtgatcgcatcgttttcaaaagcaaaaaatacgaaggtccgttcaccacgacctctgaagtcgtg |
| | (BCDRBS_alt1_ | gatatgctgagtacctatctgccgaaacagccggaaggcgtgatcctgttttacagcaaaggtccgaaatctaacatcgacttc |
| | BD6); D12 | aaaatcaaaaaagaaaacaccatcgatcaaacggccaatgttgtctttcgttatatgtcatcggaaccgattatctttggcgaaa |
| | (E. coli recode | gctctatcttcgtggaatacaaaaaattctcgaacgataaaggcttcccgaaagaatacggcagcggtaaaattgtcctgtataa |
| | 1); Twin Strep | cggtgtgaattacctgaacaatatctattgcctggaatacattaacacccataatgaagttggcattaaatctgtggttgtcccgat |
| | Tag; | caaatttattgcagaattcctggtcaacggtgaaatcctgaaaccgcgtattgacaaaaccatgaaatacatcaacagtgaagat |
| | Terminator | tactacggtaaccagcataacatcatcgtggaacacctgegcgaccaatctatcaaaatcggcgatatcttcaacgaagacaa |
| | (BBa_B0015 | actgagtgatgtcggtcaccagtatgcgaacaatgataaatttcgtctgaacccggaagtgtcctacttcaccaataaacgtacg |
| | (Double | cgcggcccgctgggtatcctgtcaaattatgtcaaaaccctgctgatttcaatgtactgttcgaaaacgtttctggatgacagcaa |
| | Terminator | caaacgcaaagttctggccattgactttggcaatggtgcagatctggaaaaatatttctacggcgaaatcgctctgctggttgcg |
| | B0010, | accgatccggacgcggatgccattgcacgtggcaacgaacgctataacaaactgaattctggtatcaaaaccaaatactacaa |
| | B0012)); | attcgactacatccaggaaaccattcgtagtgatacgttcgtgagttccgttcgcgaagtcttttatttcggcaaattcaacatcatc |
| | Terminator | gattggcaattcgccatccattattctttccatccgcgtcactacgcaaccgtgatgaacaatctgagtgaactgacggcttccg |
| | (T7 | gcggtaaagttctgattacgacgatggatggtgataaactgtccaaactgaccgataagaaaaccttcattatccacaaaaacct |
| | terminator) | gccgtcatcggaaaactacatgtcagtggaaaaaatcgccgatgaccgcattgtggtttataacccgagcacgatgtctaccc |
| | | cgatgacggaatacatcattaagaaaaacgatatcgtccgtgtgtttaatgaatacggtttcgttctggtcgacaacgttgattttg |
| | | caaccattatcgaacgcagcaaaaaattcatcaatggcgcttccacgatggaagatcgtccgtcaacgcgcaactttttcgaac |
| | | tgaatcgcggtgcaattaaatgtgaaggtctggatgtggaagatctgctgtcctattatgtcgtgtatgtgttctctaaacgctaac |
| | | cggcttatcggtcagtttcacctgatttacgtaaaaacccgcttcggcgggtttttgcttttggaggggcagaaagatgaatgact |
| | | gtccacgacgctatacccaaaagaaatgttgacaattaatcatcggctcgtataatgtgtggaattgtgagcgctcacaattgcg |
| | | aaaaatcaataaggaggcaacaagatgtgcgaaaaacatcttaatcatgcgccggaggttttctaatggatgaaatcgtcaaaa |
| | | atatccgcgaaggcacgcacgtcctgctgccgttctatgaaaccctgccggaactgaatctgtcactgggcaaatctccgctg |
| | | ccgagtctggaatatggtgcaaactactttctgcagatttctcgtgtgaacgatctgaatcgcatgccgaccgacatgctgaaac |
| | | tgttcacgcatgatatcatgctgccggaaagcgatctggacaaagtctacgaaatcctgaaaatcaactccgttaaatactacg |
| | | gccgttcaaccaaagcggatgccgtggttgcagacctgtccgctcgcaataaactgtttaaacgtgaacgcgatgctattaaat |
| | | cgaacaatcacctgaccgaaaacaacctgtacatcagcgattacaaaatgctgacgtttgacgtgttccgtccgctgttcgattt |
| | | cgttaacgaaaaatactgcatcatcaaactgccgaccctgtttggccgtggtgtgattgatacgatgcgcatctactgcagcctg |
| | | ttcaaaaatgtccgcctgctgaaatgtgtgtcggatagctggctgaaagactctgcgattatggtggccagtgacgtttgtaaga |
| | | aaaacctggacctgtttatgtcccatgtcaaatcagtgaccaaaagctctagttggaaagacgttaattcggtccaatttagcatt |
| | | ctgaacaatccggttgatacggaattcatcaacaaattcctggaattctctaaccgtgtttacgaagcactgtattacgtccacagt |
| | | ctgctgtactcctcaatgacctcggactccaaatccatcgaaaataaacatcaacgccgcctggtgaaactgctgctggggag |
| | | cgcttggagccacccgcagttcgaaaaaggtggaggttctggcggtggatcgggaggttcagcgtggagccacccgcagtt |
| | | cgagaaataaccaggcatcaaataaaacgaaaggctcagtcgaaagactgggcctttcgttttatctgttgtttgtcggtgaacg |
| | | ctctctactagagtcacactggctcaccttcggggggcctttctgcgtttataataaccccttggggcctctaaacgggtcttga |
| | | ggggttttttgc |
| 24 | Combination | aattgtgagcggataacaattacgagcttcatgcacagtgaaatcatgaaaaatttatttgctttgtgagcggataacaattataat |
| | of genetic | atgtggaattgtgagcgctcacaattccacagcgaaaaatcaataaggaggcaacaagatgtgcgaaaaacatcttaatcatg |
| | elements | cgacggagcgtttctaatgaaacatcaccatcaccatcaccccatgagegattacgacatccccactactgagaatctttattttc |
| | expressed in | agggcgccgacgccaacgtagtgagctcgtccacgattgctacatacatcgacgcactggctaaaaacgcgagtgaattaga |
| | strain 815995 | gcaacgttcaaccgcctatgaaatcaacaacgaacttgagctcgtctttattaagcctccgctaatcaccctgactaacgttgtta |
| | (Promoter | atatatctaccatccaggaaagcttcattcgcttcactgttactaacaaagaaggcgtaaaaatcaggactaaaatcccattgtct |
| | (P(T5) | aaggtgcacgggctggatgtgaaaaacgttcagctggttgacgctattgacaacatcgtatgggaaaagaaatccctcgtaac |
| | 2xlacO); RBS | cgaaaaccgtctgcataaagaatgtctgctgcgtctgagcacggaggaacgacacatctttctggattacaaaaaatatggtag |
| | (BCDRBS_alt1_ | ttctattcgtctggagctggtgaacctgatccaggcaaagaccaaaaatttcacaattgacttcaaactaaaatactttctgggctc |
| | BD18); His- | cggtgcgcagagcaaatcttccctgttgcatgctatcaaccacccgaaaagccgcccgaatacttctctggaaatcgagttcac |
| | Tag; D1 (E. | cccccgcgataacgaaactgtcccatacgatgagcttattaaggaactgaccacgctgtcccgtcacatttttatggegagccc |
| | coli recode | ggaaaacgttatattategccgcctatcaacgctccgatcaagaccttcatgttgccgaaacaagacatcgtcggtctggatctg |
| | 12); | gagaacctgtacgcagttactaaaaccgacggcatccccatcactatcagagtaacgtcaaacggattgtattgctatttcaccc |
| | Terminator | atctgggttacattattcgttacccggtgaaacgcatcatagattctgaagttgttgttttcggcgaagccgtaaaggacaaaaac |
| | (Bba_J61048); | tggaccgtctatctgatcaagctaatcgaaccggttaatgctatcaacgatcggctggaagaatcgaaatacgtagaatctaaa |
| | Promoter | ctggtggatatttgcgaccgtattgtctttaaatcgaaaaagtacgagggtcctttcactactactagcgaagtcgtggacatgct |
| | (Ptac); RBS | ctctacgtacctgccgaaacagcctgagggcgttatcctgttctatagcaaaggtccgaaatccaacatcgattttaagattaaa |
| | (BCDRBS_alt4_B | aaggaaaacaccattgatcagacggctaatgtagttttccggtacatgtctagcgagccgatcatctttggcgaatcttctatcttt |
| | D15); D12 | gtagaatataaaaagttcagcaacgacaaaggattcccaaaagaatacgggtccgggaaaatcgtcttatacaacggtgttaa |
| | (E. coli recode | ctacttgaacaacatctattgcctggaatatatcaatactcacaatgaagttggtattaaatcagtggttgttccgataaaattcatc |
| | 2); Twin Strep | gcggaatttctggtcaatggcgaaatcctgaaaccccgcattgataagaccatgaaatacataaactccgaagactactacggt |
| | Tag; | aaccagcataacatcatcgtggaacacctgagagatcagagtatcaaaatcggcgacattttcaatgaggacaagttaagcga |
| | Terminator | cgtgggccatcaatacgcaaacaacgacaaattccgtctgaacccggaggtttcctatttcaccaacaaacgtacccgaggtc |
| | (BBa_B0015 | cgcttggcatcctctccaattacgtaaaaaccctgctgatttctatgtattgttcaaaaacgttcctggatgacagcaacaaaagg |
| | (Double | aaggtactggctatcgatttcggtaacggcgcggatctggaaaagtacttttacggtgaaatcgctctgttagtcgcaactgatc |
| | Terminator | cggacgccgacgcaattgctcgcggaaatgaacgttacaacaaactgaactccggtattaaaacaaagtattataaattcgact |
| | B0010, | atatccaggagactatccgctctgatactttcgtgagcagcgtgcgtgaggttttttactttggtaaattcaacattattgactggca |
| | B0012)); | gtttgcgatccactacagctttcacccgcgtcactatgcgaccgttatgaataacctateggaactcacggctagcggcggcaa |
| | Terminator | agtgctgattactactatggacggtgacaaactgtctaagctgaccgataagaaaaccttcatcatccacaaaaacttgccaagt |
| | (T7 | tctgagaactatatgtctgttgaaaaaattgcggacgaccgcatcgtcgtttacaacccatctaccatgtccacccctatgacag |
| | terminator) | agtacatcatcaaaaagaacgacatagttcgtgttttcaacgaatacggcttcgtactggtagataacgtcgattttgctaccatta |
| | | tcgagcgttcgaaaaaattcattaacggtgcttccactatggaagatcgtccgtccactcgtaacttttttgaattaaaccgtggcg |
| | | caatcaaatgcgaagggctggatgtggaagacctcctgtcttactacgttgtatacgtcttctctaaacgctaaccggcttatcgg |
| | | tcagtttcacctgatttacgtaaaaacccgcttcgggggtttttgcttttggaggggcagaaagatgaatgactgtccacgacg |
| | | ctatacccaaaagaaatgttgacaattaatcatcggctcgtataatgtgtggaattgtgagcgctcacaattgtcaataaaggcat |
| | | ataaaaggaggttaataacatgaaagttaaagtaaaacatcttaatcatgcgggggagtctttctaatggatgagatcgttaaga |
| | | acattcgtgaaggtacgcatgtgcttttgccattttacgaaactctcccggaactgaatctgtccttaggcaaaagccctctaccc |
| | | tctctggagtatggggccaactacttcctgcaaatctcacgcgtcaacgacctgaatcgaatgccgaccgacatgctgaaact |
| | | gttcactcacgatataatgctgccggaaagtgatctggacaaagtatatgaaatcctgaaaatcaacagcgttaagtactacgg |
| | | acggtcgaccaaagcggacgctgttgtagcagatctgtctgctcgcaacaaactctttaaacgtgaacgtgacgctattaagtc |
| | | caacaaccacctgacagagaacaatctctatatctctgactacaaaatgttgactttcgatgtgttccgtccgctgtttgatttcgtg |
| | | aacgaaaaatattgcattatcaaactgccgaccctgttcggccgtggtgttattgacaccatgcgcatctactgtagcctcttcaa |
| | | gaatgtcagactactgaaatgcgtgtccgatagctggctgaaagacagcgcaatcatggtagcctcagacgtttgcaaaaaga |
| | | acctggatctgtttatgtcccatgttaaatccgttactaagtctagctcgtggaaagatgttaacagegtacagttttctattttgaac |
| | | aaccctgttgacacggaatttatcaacaaattcctggagttctctaaccgtgtatacgaagcgctgtattacgtgcactccttactg |
| | | tactcttctatgaccagcgatagtaagtctatcgaaaataaacaccagcgccgtctggtaaaactgctccttgggagcgcttgga |
| | | gccacccgcagttcgaaaaaggtggaggttctggcggtggatcgggaggttcagcgtggagccacccgcagttcgagaaat |
| | | aaccaggcatcaaataaaacgaaaggctcagtcgaaagactgggcctttcgttttatctgttgtttgtcggtgaacgctctctact |
| | | agagtcacactggctcaccttcgggtgggcctttctgcgtttataataaccccttggggcctctaaacgggtcttgaggggtttttt |
| | | gc |
| 25 | Combination | aattgtgagcggataacaattacgagcttcatgcacagtgaaatcatgaaaaatttatttgctttgtgagcggataacaattataat |
| | of genetic | atgtggaattgtgagcgctcacaattccacagcgaaaaatcaataaggaggcaacaagatgtgcgaaaaacatcttaatcatg |
| | elements | cacaggagactttctaatgaaacatcaccatcaccatcaccccatgagegattacgacatccccactactgagaatctttattttc |
| | expressed in | agggcgccgacgccaacgtagtgagctcgtccacgattgctacatacatcgacgcactggctaaaaacgcgagtgaattaga |
| | strain 816008 | gcaacgttcaaccgcctatgaaatcaacaacgaacttgagctcgtctttattaagcctccgctaatcaccctgactaacgttgtta |
| | (Promoter | atatatctaccatccaggaaagcttcattcgcttcactgttactaacaaagaaggcgtaaaaatcaggactaaaatcccattgtct |
| | (P(T5) | aaggtgcacgggctggatgtgaaaaacgttcagctggttgacgctattgacaacatcgtatgggaaaagaaatccctcgtaac |
| | 2xlacO); RBS | cgaaaaccgtctgcataaagaatgtctgctgcgtctgagcacggaggaacgacacatctttctggattacaaaaaatatggtag |
| | (BCDRBS_alt1_ | ttctattcgtctggagctggtgaacctgatccaggcaaagaccaaaaatttcacaattgacttcaaactaaaatactttctgggctc |
| | BD1); His- | cggtgcgcagagcaaatcttccctgttgcatgctatcaaccacccgaaaagccgcccgaatacttctctggaaatcgagttcac |
| | Tag; D1 (E. | cccccgcgataacgaaactgtcccatacgatgagcttattaaggaactgaccacgctgtcccgtcacatttttatggegagecc |
| | coli recode 12) | ggaaaacgttatattatcgccgcctatcaacgctccgatcaagaccttcatgttgccgaaacaagacatcgtcggtctggatctg |
| | Terminator | gagaacctgtacgcagttactaaaaccgacggcatccccatcactatcagagtaacgtcaaacggattgtattgctatttcaccc |
| | (Bba_J61048); | atctgggttacattattcgttacccggtgaaacgcatcatagattctgaagttgttgttttcggcgaagccgtaaaggacaaaaac |
| | Promoter | tggaccgtctatctgatcaagctaatcgaaccggttaatgctatcaacgatcggctggaagaatcgaaatacgtagaatctaaa |
| | (Ptac); RBS | ctggtggatatttgcgaccgtattgtctttaaatcgaaaaagtacgagggtcctttcactactactagcgaagtcgtggacatgct |
| | (BCDRBS_alt4_ | ctctacgtacctgccgaaacagcctgagggcgttatcctgttctatagcaaaggtccgaaatccaacatcgattttaagattaaa |
| | BD2); D12 | aaggaaaacaccattgatcagacggctaatgtagttttccggtacatgtctagcgagccgatcatctttggcgaatcttctatcttt |
| | (E. coli recode | gtagaatataaaaagttcagcaacgacaaaggattcccaaaagaatacgggtccgggaaaatcgtcttatacaacggtgttaa |
| | 2); Twin Strep | ctacttgaacaacatctattgcctggaatatatcaatactcacaatgaagttggtattaaatcagtggttgttccgataaaattcatc |
| | Tag; | gcggaatttctggtcaatggcgaaatcctgaaaccccgcattgataagaccatgaaatacataaactccgaagactactacggt |
| | Terminator | aaccagcataacatcatcgtggaacacctgagagatcagagtatcaaaatcggcgacattttcaatgaggacaagttaagcga |
| | (BBa_B0015 | cgtgggccatcaatacgcaaacaacgacaaattccgtctgaacccggaggtttcctatttcaccaacaaacgtacccgaggtc |
| | (Double | cgcttggcatcctctccaattacgtaaaaaccctgctgatttctatgtattgttcaaaaacgttcctggatgacagcaacaaaagg |
| | Terminator | aaggtactggctatcgatttcggtaacggcgcggatctggaaaagtacttttacggtgaaatcgctctgttagtcgcaactgatc |
| | B0010, | cggacgccgacgcaattgctcgcggaaatgaacgttacaacaaactgaactccggtattaaaacaaagtattataaattcgact |
| | B0012)); | atatccaggagactatccgctctgatactttcgtgagcagcgtgcgtgaggttttttactttggtaaattcaacattattgactggca |
| | Terminator | gtttgcgatccactacagctttcacccgcgtcactatgcgaccgttatgaataacctatcggaactcacggctagcggcggcaa |
| | (T7 | agtgctgattactactatggacggtgacaaactgtctaagctgaccgataagaaaaccttcatcatccacaaaaacttgccaagt |
| | terminator) | tctgagaactatatgtctgttgaaaaaattgcggacgaccgcatcgtcgtttacaacccatctaccatgtccacccctatgacag |
| | | agtacatcatcaaaaagaacgacatagttcgtgttttcaacgaatacggcttcgtactggtagataacgtcgattttgctaccatta |
| | | tcgagcgttcgaaaaaattcattaacggtgcttccactatggaagatcgtccgtccactcgtaacttttttgaattaaaccgtggcg |
| | | caatcaaatgcgaagggctggatgtggaagacctcctgtcttactacgttgtatacgtcttctctaaacgctaaccggcttatcgg |
| | | tcagtttcacctgatttacgtaaaaacccgcttcggcgggtttttgcttttggaggggcagaaagatgaatgactgtccacgacg |
| | | ctatacccaaaagaaatgttgacaattaatcatcggctcgtataatgtgtggaattgtgagcgctcacaattgtcaataaaggcat |
| | | ataaaaggaggttaataacatgaaagttaaagtaaaacatcttaatcatgctaaggaggttttctaatggatgagatcgttaagaa |
| | | cattcgtgaaggtacgcatgtgcttttgccattttacgaaactctcccggaactgaatctgtccttaggcaaaagccctctaccct |
| | | ctctggagtatggggccaactacttcctgcaaatctcacgcgtcaacgacctgaatcgaatgccgaccgacatgctgaaactg |
| | | ttcactcacgatataatgctgccggaaagtgatctggacaaagtatatgaaatcctgaaaatcaacagcgttaagtactacgga |
| | | cggtcgaccaaagcggacgctgttgtagcagatctgtctgctcgcaacaaactctttaaacgtgaacgtgacgctattaagtcc |
| | | aacaaccacctgacagagaacaatctctatatctctgactacaaaatgttgactttcgatgtgttccgtccgctgtttgatttcgtga |
| | | acgaaaaatattgcattatcaaactgccgaccctgttcggccgtggtgttattgacaccatgcgcatctactgtagcctcttcaag |
| | | aatgtcagactactgaaatgcgtgtccgatagctggctgaaagacagcgcaatcatggtagcctcagacgtttgcaaaaagaa |
| | | cctggatctgtttatgtcccatgttaaatccgttactaagtctagctcgtggaaagatgttaacagcgtacagttttctattttgaaca |
| | | accctgttgacacggaatttatcaacaaattcctggagttctctaaccgtgtatacgaagcgctgtattacgtgcactecttactgt |
| | | actcttctatgaccagcgatagtaagtctatcgaaaaaaacaccagcgccgtctggtaaaactgctccttgggagcgcttgga |
| | | gccacccgcagttcgaaaaaggtggaggttctggcggtggatcgggaggttcagcgtggagccacccgcagttcgagaaat |
| | | aaccaggcatcaaataaaacgaaaggctcagtcgaaagactgggcctttcgttttatctgttgtttgtcggtgaacgctctctact |
| | | agagtcacactggctcaccttcggggggcctttctgcgtttataataaccccttggggcctctaaacgggtcttgaggggtttttt |
| | | gc |
| 26 | Combination | aattgtgagcggataacaattacgagcttcatgcacagtgaaatcatgaaaaatttatttgctttgtgagcggataacaattataat |
| | of genetic | atgtggaattgtgagcgctcacaattccacagcgaaaaatcaataaggaggcaacaagatgtgcgaaaaacatcttaatcatg |
| | elements | cggtggagggtttctaatgaaacatcaccatcaccatcaccccatgagegattacgacatccccactactgagaatctttattttc |
| | expressed in | agggcgccgacgctaatgtcgtgtcttcttctaccatcgcaacctatattgacgctctggcaaaaaacgcctcggaactggaac |
| | strain 816056 | aacgctcaaccgcgtatgaaatcaacaatgaactggaactggtgtttatcaaaccgccgctgattacgctgaccaacgtggtta |
| | (Promoter | atatcagcaccattcaggaatcttttattcgtttcacggttaccaacaaagaaggcgtcaaaatccgcacgaaaattccgctgag |
| | (P(T5) | caaagttcatggtctggatgtgaaaaacgttcaactggtcgacgcaatcgataatattgtgtgggaaaagaaaagcctggttac |
| | 2xlacO); RBS | cgaaaatcgtctgcataaagaatgcctgctgcgtctgagcacggaagaacgccacatctttctggactataaaaaatacggca |
| | (BCDRBS_alt1_ | gctctatccgcctggaactggtgaacctgatccaggctaaaaccaaaaacttcacgatcgatttcaaactgaaatattttctggg |
| | BD14); His- | cagtggtgctcaatccaaaagttccctgctgcatgcgatcaaccacccgaaaagtcgtccgaatacctccctggaaattgaatt |
| | Tag; D1 (E. | caccccgcgcgacaacgaaacggtgccgtacgatgaactgattaaagaactgaccacgctgtcacgtcatatctttatggcgt |
| | coli recode 1); | cgccggaaaacgttattctgagcccgccgatcaatgccccgattaaaaccttcatgctgccgaaacaggacattgttggcctg |
| | Terminator | gatctggaaaacctgtatgcggtcacgaaaaccgatggtattccgatcaccattcgcgtgacgtcgaatggcctgtattgctact |
| | (Bba_J61048); | ttacccacctgggttatattatccgttacccggttaaacgcattatcgactccgaagtcgtggttttcggcgaagcggtcaaagat |
| | Promoter | aaaaattggaccgtgtatctgatcaaactgattgaaccggtgaacgccatcaacgatcgtctggaagaatcaaaatacgtgga |
| | (Ptac); RBS | atcgaaactggttgacatctgtgatcgcatcgttttcaaaagcaaaaaatacgaaggtccgttcaccacgacctctgaagtcgtg |
| | (BCDRBS_alt1_ | gatatgctgagtacctatctgccgaaacagccggaaggcgtgatcctgttttacagcaaaggtccgaaatctaacatcgacttc |
| | BD6); D12 | aaaatcaaaaaagaaaacaccatcgatcaaacggccaatgttgtctttcgttatatgtcatcggaaccgattatctttggcgaaa |
| | (E. coli recode | gctctatcttcgtggaatacaaaaaattctcgaacgataaaggcttcccgaaagaatacggcagcggtaaaattgtcctgtataa |
| | 1); Twin Strep | cggtgtgaattacctgaacaatatctattgcctggaatacattaacacccataatgaagttggcattaaatctgtggttgtcccgat |
| | Tag; | caaatttattgcagaattcctggtcaacggtgaaatcctgaaaccgcgtattgacaaaaccatgaaatacatcaacagtgaagat |
| | Terminator | tactacggtaaccagcataacatcatcgtggaacacctgcgcgaccaatctatcaaaatcggcgatatcttcaacgaagacaa |
| | (BBa_B0015 | actgagtgatgtcggtcaccagtatgcgaacaatgataaatttcgtctgaacccggaagtgtcctacttcaccaataaacgtacg |
| | (Double | cgcggcccgctgggtatcctgtcaaattatgtcaaaaccctgctgatttcaatgtactgttcgaaaacgtttctggatgacagcaa |
| | Terminator | caaacgcaaagttctggccattgactttggcaatggtgcagatctggaaaaatatttctacggcgaaatcgctctgctggttgcg |
| | B0010, | accgatccggacgcggatgccattgcacgtggcaacgaacgctataacaaactgaattctggtatcaaaaccaaatactacaa |
| | B0012)); | attcgactacatccaggaaaccattcgtagtgatacgttcgtgagttccgttcgcgaagtcttttatttcggcaaattcaacatcatc |
| | Terminator | gattggcaattcgccatccattattctttccatccgcgtcactacgcaaccgtgatgaacaatctgagtgaactgacggcttccg |
| | (T7 | gcggtaaagttctgattacgacgatggatggtgataaactgtccaaactgaccgataagaaaaccttcattatccacaaaaacct |
| | terminator) | gccgtcatcggaaaactacatgtcagtggaaaaaatcgccgatgaccgcattgtggtttataacccgagcacgatgtctaccc |
| | Combination | cgatgacggaatacatcattaagaaaaacgatatcgtccgtgtgtttaatgaatacggtttcgttctggtcgacaacgttgattttg |
| | | caaccattatcgaacgcagcaaaaaattcatcaatggcgcttccacgatggaagatcgtccgtcaacgcgcaactttttcgaac |
| | | tgaatcgcggtgcaattaaatgtgaaggtctggatgtggaagatctgctgtcctattatgtcgtgtatgtgttctctaaacgctaac |
| | | cggcttatcggtcagtttcacctgatttacgtaaaaacccgcttcggcgggtttttgcttttggaggggcagaaagatgaatgact |
| | | gtccacgacgctatacccaaaagaaatgttgacaattaatcatcggctcgtataatgtgtggaattgtgagcgctcacaattgcg |
| | | aaaaatcaataaggaggcaacaagatgtgcgaaaaacatcttaatcatgcgccggaggttttctaatggatgaaatcgtcaaaa |
| | | atatccgcgaaggcacgcacgtcctgctgccgttctatgaaaccctgccggaactgaatctgtcactgggcaaatctccgctg |
| | | ccgagtctggaatatggtgcaaactactttctgcagatttctcgtgtgaacgatctgaatcgcatgccgaccgacatgctgaaac |
| | | tgttcacgcatgatatcatgctgccggaaagcgatctggacaaagtctacgaaatcctgaaaatcaactccgttaaatactacg |
| | | gccgttcaaccaaagcggatgccgtggttgcagacctgtccgctcgcaataaactgtttaaacgtgaacgcgatgctattaaat |
| | | cgaacaatcacctgaccgaaaacaacctgtacatcagegattacaaaatgctgacgtttgacgtgttccgtccgctgttcgattt |
| | | cgttaacgaaaaatactgcatcatcaaactgccgaccctgtttggccgtggtgtgattgatacgatgcgcatctactgcagcctg |
| | | ttcaaaaatgtccgcctgctgaaatgtgtgtcggatagctggctgaaagactctgcgattatggtggccagtgacgtttgtaaga |
| | | aaaacctggacctgtttatgtcccatgtcaaatcagtgaccaaaagctctagttggaaagacgttaattcggtccaatttagcatt |
| | | ctgaacaatccggttgatacggaattcatcaacaaattcctggaattctctaaccgtgtttacgaagcactgtattacgtccacagt |
| | | ctgctgtactcctcaatgacctcggactccaaatccatcgaaaataaacatcaacgccgcctggtgaaactgctgctggggag |
| | | cgcttggagccacccgcagttcgaaaaaggtggaggttctggcggtggatcgggaggttcagcgtggagccacccgcagtt |
| | | cgagaaataaccaggcatcaaataaaacgaaaggctcagtcgaaagactgggcctttcgttttatctgttgtttgtcggtgaacg |
| | | ctctctactagagtcacactggctcaccttcggggggcctttctgcgtttataataaccccttggggcctctaaacgggtcttga |
| | | ggggttttttgc |
| 27 | Combination | aattgtgagcggataacaattacgagcttcatgcacagtgaaatcatgaaaaatttatttgctttgtgagcggataacaattataat |
| | of genetic | atgtggaattgtgagcgctcacaattccacagcgaaaaatcaataaggaggcaacaagatgtgcgaaaaacatcttaatcatg |
| | elements | cggaggatcgtttctaatgaaacatcaccatcaccatcaccccatgagegattacgacatccccactactgagaatctttattttc |
| | expressed in | agggcgccgacgctaatgtcgtgtcttcttctaccatcgcaacctatattgacgctctggcaaaaaacgcctcggaactggaac |
| | strain 816070 | aacgctcaaccgcgtatgaaatcaacaatgaactggaactggtgtttatcaaaccgccgctgattacgctgaccaacgtggtta |
| | (Promoter | atatcagcaccattcaggaatcttttattcgtttcacggttaccaacaaagaaggcgtcaaaatccgcacgaaaattccgctgag |
| | (P(T5) | caaagttcatggtctggatgtgaaaaacgttcaactggtcgacgcaatcgataatattgtgtgggaaaagaaaagcctggttac |
| | 2xlacO); RBS | cgaaaatcgtctgcataaagaatgcctgctgcgtctgagcacggaagaacgccacatctttctggactataaaaaatacggca |
| | (BCDRBS_alt1_ | gctctatccgcctggaactggtgaacctgatccaggctaaaaccaaaaacttcacgatcgatttcaaactgaaatattttctggg |
| | BD10); His- | cagtggtgctcaatccaaaagttccctgctgcatgcgatcaaccacccgaaaagtcgtccgaatacctccctggaaattgaatt |
| | Tag; D1 (E. | caccccgcgcgacaacgaaacggtgccgtacgatgaactgattaaagaactgaccacgctgtcacgtcatatctttatggcgt |
| | coli recode 1); | cgccggaaaacgttattctgagcccgccgatcaatgccccgattaaaaccttcatgctgccgaaacaggacattgttggcctg |
| | Terminator | gatctggaaaacctgtatgcggtcacgaaaaccgatggtattccgatcaccattcgcgtgacgtcgaatggcctgtattgctact |
| | (Bba_J61048); | ttacccacctgggttatattatccgttacccggttaaacgcattatcgactccgaagtcgtggttttcggcgaagcggtcaaagat |
| | (Promoter | aaaaattggaccgtgtatctgatcaaactgattgaaccggtgaacgccatcaacgatcgtctggaagaatcaaaatacgtgga |
| | (Ptac); RBS | atcgaaactggttgacatctgtgatcgcatcgttttcaaaagcaaaaaatacgaaggtccgttcaccacgacctctgaagtcgtg |
| | (BCDRBS_alt4_ | gatatgctgagtacctatctgccgaaacagccggaaggcgtgatcctgttttacagcaaaggtccgaaatctaacatcgacttc |
| | BD15); D12 | aaaatcaaaaaagaaaacaccatcgatcaaacggccaatgttgtctttcgttatatgtcatcggaaccgattatctttggcgaaa |
| | (E. coli recode | gctctatcttcgtggaatacaaaaaattctcgaacgataaaggcttcccgaaagaatacggcagcggtaaaattgtcctgtataa |
| | 1); Twin Strep | cggtgtgaattacctgaacaatatctattgcctggaatacattaacacccataatgaagttggcattaaatctgtggttgtcccgat |
| | Tag; | caaatttattgcagaattcctggtcaacggtgaaatcctgaaaccgcgtattgacaaaaccatgaaatacatcaacagtgaagat |
| | Terminator | tactacggtaaccagcataacatcatcgtggaacacctgcgcgaccaatctatcaaaatcggcgatatcttcaacgaagacaa |
| | (BBa_B0015 | actgagtgatgtcggtcaccagtatgcgaacaatgataaatttcgtctgaacccggaagtgtcctacttcaccaataaacgtacg |
| | (Double | cgcggcccgctgggtatcctgtcaaattatgtcaaaaccctgctgatttcaatgtactgttcgaaaacgtttctggatgacagcaa |
| | Terminator | caaacgcaaagttctggccattgactttggcaatggtgcagatctggaaaaatatttctacggcgaaatcgctctgctggttgcg |
| | B0010, | accgatccggacgcggatgccattgcacgtggcaacgaacgctataacaaactgaattctggtatcaaaaccaaatactacaa |
| | B0012)); | attcgactacatccaggaaaccattcgtagtgatacgttcgtgagttccgttcgcgaagtcttttatttcggcaaattcaacatcatc |
| | Terminator | gattggcaattcgccatccattattctttccatccgcgtcactacgcaaccgtgatgaacaatctgagtgaactgacggcttccg |
| | (T7 | gcggtaaagttctgattacgacgatggatggtgataaactgtccaaactgaccgataagaaaaccttcattatccacaaaaacct |
| | terminator) | gccgtcatcggaaaactacatgtcagtggaaaaaatcgccgatgaccgcattgtggtttataacccgagcacgatgtctaccc |
| | | cgatgacggaatacatcattaagaaaaacgatatcgtccgtgtgtttaatgaatacggtttcgttctggtcgacaacgttgattttg |
| | | caaccattatcgaacgcagcaaaaaattcatcaatggcgcttccacgatggaagatcgtccgtcaacgcgcaactttttcgaac |
| | | tgaatcgcggtgcaattaaatgtgaaggtctggatgtggaagatctgctgtcctattatgtcgtgtatgtgttctctaaacgctaac |
| | | cggcttatcggtcagtttcacctgatttacgtaaaaacccgcttcggcgggtttttgcttttggaggggcagaaagatgaatgact |
| | | gtccacgacgctatacccaaaagaaatgttgacaattaatcatcggctcgtataatgtgtggaattgtgagcgctcacaattgtc |
| | | aataaaggcatataaaaggaggttaataacatgaaagttaaagtaaaacatcttaatcatgcgggggagtctttctaatggatga |
| | | aatcgtcaaaaatatccgcgaaggcacgcacgtcctgctgccgttctatgaaaccctgccggaactgaatctgtcactgggca |
| | | aatctccgctgccgagtctggaatatggtgcaaactactttctgcagatttctcgtgtgaacgatctgaatcgcatgccgaccga |
| | | catgctgaaactgttcacgcatgatatcatgctgccggaaagcgatctggacaaagtctacgaaatcctgaaaatcaactccgt |
| | | taaatactacggccgttcaaccaaagcggatgccgtggttgcagacctgtccgctcgcaataaactgtttaaacgtgaacgcg |
| | | atgctattaaatcgaacaatcacctgaccgaaaacaacctgtacatcagcgattacaaaatgctgacgtttgacgtgttccgtcc |
| | | gctgttcgatttcgttaacgaaaaatactgcatcatcaaactgccgaccctgtttggccgtggtgtgattgatacgatgcgcatct |
| | | actgcagcctgttcaaaaatgtccgcctgctgaaatgtgtgtcggatagctggctgaaagactctgcgattatggtggccagtg |
| | | acgtttgtaagaaaaacctggacctgtttatgtcccatgtcaaatcagtgaccaaaagctctagttggaaagacgttaattcggtc |
| | | caatttagcattctgaacaatccggttgatacggaattcatcaacaaattcctggaattctctaaccgtgtttacgaagcactgtatt |
| | | acgtccacagtctgctgtactcctcaatgaccteggactccaaatccatcgaaaataaacatcaacgccgcctggtgaaactgc |
| | | tgctggggagcgcttggagccacccgcagttcgaaaaaggtggaggttctggcggtggatcgggaggttcagcgtggagc |
| | | cacccgcagttcgagaaataaccaggcatcaaataaaacgaaaggctcagtcgaaagactgggcctttcgttttatctgttgttt |
| | | gtcggtgaacgctctctactagagtcacactggctcaccttcggggggcctttctgcgtttataataaccccttggggcctctaa |
| | | acgggtcttgaggggttttttgc |
| 28 | Combination | aattgtgagcggataacaattacgagcttcatgcacagtgaaatcatgaaaaatttatttgctttgtgagcggataacaattataat |
| | of genetic | atgtggaattgtgagcgctcacaattccacagcgaaaaatcaataaggaggcaacaagatgtgcgaaaaacatcttaatcatg |
| | elements | cggaggatcgtttctaatgaaacatcaccatcaccatcaccccatgagegattacgacatccccactactgagaatctttattttc |
| | expressed in | agggcgccgacgctaatgtcgtgtcttcttctaccatcgcaacctatattgacgctctggcaaaaaacgcctcggaactggaac |
| | strain 816072 | aacgctcaaccgcgtatgaaatcaacaatgaactggaactggtgtttatcaaaccgccgctgattacgctgaccaacgtggtta |
| | (Promoter | atatcagcaccattcaggaatcttttattcgtttcacggttaccaacaaagaaggcgtcaaaatccgcacgaaaattccgctgag |
| | (P(T5) | caaagttcatggtctggatgtgaaaaacgttcaactggtcgacgcaatcgataatattgtgtgggaaaagaaaagcctggttac |
| | 2xlacO); RBS | cgaaaatcgtctgcataaagaatgcctgctgcgtctgagcacggaagaacgccacatctttctggactataaaaaatacggca |
| | (BCDRBS_alt1_ | gctctatccgcctggaactggtgaacctgatccaggctaaaaccaaaaacttcacgatcgatttcaaactgaaatattttctggg |
| | BD10); His- | cagtggtgctcaatccaaaagttccctgctgcatgcgatcaaccacccgaaaagtcgtccgaatacctccctggaaattgaatt |
| | Tag; D1 (E. | caccccgcgcgacaacgaaacggtgccgtacgatgaactgattaaagaactgaccacgctgtcacgtcatatctttatggcgt |
| | coli recode 1); | cgccggaaaacgttattctgagcccgccgatcaatgccccgattaaaaccttcatgctgccgaaacaggacattgttggcctg |
| | Terminator | gatctggaaaacctgtatgcggtcacgaaaaccgatggtattccgatcaccattcgcgtgacgtcgaatggcctgtattgctact |
| | (Bba_J61048); | ttacccacctgggttatattatccgttacccggttaaacgcattatcgactccgaagtcgtggttttcggcgaagcggtcaaagat |
| | Promoter | aaaaattggaccgtgtatctgatcaaactgattgaaccggtgaacgccatcaacgatcgtctggaagaatcaaaatacgtgga |
| | (Ptac); RBS | atcgaaactggttgacatctgtgatcgcatcgttttcaaaagcaaaaaatacgaaggtccgttcaccacgacctctgaagtcgtg |
| | (BCDRBS_alt4_ | gatatgctgagtacctatctgccgaaacagccggaaggcgtgatcctgttttacagcaaaggtccgaaatctaacatcgacttc |
| | BD11); D12 | aaaatcaaaaaagaaaacaccatcgatcaaacggccaatgttgtctttcgttatatgtcatcggaaccgattatctttggcgaaa |
| | (E. coli recode | gctctatcttcgtggaatacaaaaaattctcgaacgataaaggcttcccgaaagaatacggcagcggtaaaattgtcctgtataa |
| | 1); Twin Strep | cggtgtgaattacctgaacaatatctattgcctggaatacattaacacccataatgaagttggcattaaatctgtggttgtcccgat |
| | Tag; | caaatttattgcagaattcctggtcaacggtgaaatcctgaaaccgcgtattgacaaaaccatgaaatacatcaacagtgaagat |
| | Terminator | tactacggtaaccagcataacatcatcgtggaacacctgcgcgaccaatctatcaaaatcggcgatatcttcaacgaagacaa |
| | (BBa_B0015 | actgagtgatgtcggtcaccagtatgcgaacaatgataaatttcgtctgaacccggaagtgtcctacttcaccaataaacgtacg |
| | (Double | cgcggcccgctgggtatcctgtcaaattatgtcaaaaccctgctgatttcaatgtactgttcgaaaacgtttctggatgacagcaa |
| | Terminator | caaacgcaaagttctggccattgactttggcaatggtgcagatctggaaaaatatttctacggcgaaatcgctctgctggttgcg |
| | B0010, | accgatccggacgcggatgccattgcacgtggcaacgaacgctataacaaactgaattctggtatcaaaaccaaatactacaa |
| | B0012)); | attcgactacatccaggaaaccattcgtagtgatacgttcgtgagttccgttcgcgaagtcttttatttcggcaaattcaacatcatc |
| | Terminator | gattggcaattcgccatccattattctttccatccgcgtcactacgcaaccgtgatgaacaatctgagtgaactgacggcttccg |
| | (T7 | gcggtaaagttctgattacgacgatggatggtgataaactgtccaaactgaccgataagaaaaccttcattatccacaaaaacct |
| | terminator) | gccgtcatcggaaaactacatgtcagtggaaaaaatcgccgatgaccgcattgtggtttataacccgagcacgatgtctaccc |
| | | cgatgacggaatacatcattaagaaaaacgatatcgtccgtgtgtttaatgaatacggtttcgttctggtcgacaacgttgattttg |
| | | caaccattatcgaacgcagcaaaaaattcatcaatggcgcttccacgatggaagatcgtccgtcaacgcgcaactttttcgaac |
| | | tgaatcgcggtgcaattaaatgtgaaggtctggatgtggaagatctgctgtcctattatgtcgtgtatgtgttctctaaacgctaac |
| | | cggcttatcggtcagtttcacctgatttacgtaaaaacccgcttcgggggtttttgcttttggaggggcagaaagatgaatgact |
| | | gtccacgacgctatacccaaaagaaatgttgacaattaatcatcggctcgtataatgtgtggaattgtgagcgctcacaattgtc |
| | | aataaaggcatataaaaggaggttaataacatgaaagttaaagtaaaacatcttaatcatgcgggggagtgtttctaatggatga |
| | | aatcgtcaaaaatatccgcgaaggcacgcacgtcctgctgccgttctatgaaaccctgccggaactgaatctgtcactgggca |
| | | aatctccgctgccgagtctggaatatggtgcaaactactttctgcagatttctcgtgtgaacgatctgaatcgcatgccgaccga |
| | | catgctgaaactgttcacgcatgatatcatgctgccggaaagcgatctggacaaagtctacgaaatcctgaaaatcaactccgt |
| | | taaatactacggccgttcaaccaaagcggatgccgtggttgcagacctgtccgctcgcaataaactgtttaaacgtgaacgcg |
| | | atgctattaaatcgaacaatcacctgaccgaaaacaacctgtacatcagegattacaaaatgctgacgtttgacgtgttccgtcc |
| | | gctgttcgatttcgttaacgaaaaatactgcatcatcaaactgccgaccctgtttggccgtggtgtgattgatacgatgcgcatct |
| | | actgcagcctgttcaaaaatgtccgcctgctgaaatgtgtgtcggatagctggctgaaagactctgcgattatggtggccagtg |
| | | acgtttgtaagaaaaacctggacctgtttatgtcccatgtcaaatcagtgaccaaaagctctagttggaaagacgttaatteggtc |
| | | caatttagcattctgaacaatccggttgatacggaattcatcaacaaattcctggaattctctaaccgtgtttacgaagcactgtatt |
| | | acgtccacagtctgctgtactcctcaatgacctcggactccaaatccatcgaaaaaaacatcaacgccgcctggtgaaactgc |
| | | tgctggggagcgcttggagccacccgcagttcgaaaaaggtggaggttctggcggtggatcgggaggttcagcgtggagc |
| | | cacccgcagttcgagaaataaccaggcatcaaataaaacgaaaggctcagtcgaaagactgggcctttcgttttatctgttgttt |
| | | gtcggtgaacgctctctactagagtcacactggctcaccttcggggggcctttctgcgtttataataaccccttggggcctctaa |
| | | acgggtcttgaggggttttttgc |
| 29 | D1 amino acid | MDANVVSSSTIATYIDALAKNASELEQRSTAYEINNELELVFIKPPLITLTNVVNI |
| | sequence | STIQESFIRFTVTNKEGVKIRTKIPLSKVHGLDVKNVQLVDAIDNIVWEKKSLVT |
| | (Uniprot | ENRLHKECLLRLSTEERHIFLDYKKYGSSIRLELVNLIQAKTKNFTIDFKLKYFL |
| | Accession No. | GSGAQSKSSLLHAINHPKSRPNTSLEIEFTPRDNETVPYDELIKELTTLSRHIFMA |
| | P04298) | SPENVILSPPINAPIKTFMLPKQDIVGLDLENLYAVTKTDGIPITIRVTSNGLYCYF |
| | | THLGYIIRYPVKRIIDSEVVVFGEAVKDKNWTVYLIKLIEPVNAINDRLEESKYV |
| | | ESKLVDICDRIVFKSKKYEGPFTTTSEVVDMLSTYLPKQPEGVILFYSKGPKSNI |
| | | DFKIKKENTIDQTANVVFRYMSSEPIIFGESSIFVEYKKFSNDKGFPKEYGSGKIV |
| | | LYNGVNYLNNIYCLEYINTHNEVGIKSVVVPIKFIAEFLVNGEILKPRIDKTMKYI |
| | | NSEDYYGNQHNIIVEHLRDQSIKIGDIFNEDKLSDVGHQYANNDKFRLNPEVSY |
| | | FTNKRTRGPLGILSNYVKTLLISMYCSKTFLDDSNKRKVLAIDFGNGADLEKYF |
| | | YGEIALLVATDPDADAIARGNERYNKLNSGIKTKYYKFDYIQETIRSDTFVSSVR |
| | | EVFYFGKFNIIDWQFAIHYSFHPRHYATVMNNLSELTASGGKVLITTMDGDKLS |
| | | KLTDKKTFIIHKNLPSSENYMSVEKIADDRIVVYNPSTMSTPMTEYIIKKNDIVR |
| | | VFNEYGFVLVDNVDFATIIERSKKFINGASTMEDRPSTRNFFELNRGAIKCEGLD |
| | | VEDLLSYYVVYVFSKR |
| 30 | D1 nucleotide | atggatgccaacgtagtatcatcttctactattgcgacgtatatagacgctttagcgaagaatgcttcggaattagaacagaggtc |
| | sequence | taccgcatacgaaataaataatgaattggaactagtatttattaagccgccattgattactttgacaaatgtagtgaatatctctacg |
| | (NCBI | attcaggaatcgtttattcgatttaccgttactaataaggaaggtgttaaaattagaactaagattccattatctaaggtacatggtct |
| | Reference | agatgtaaaaaatgtacagttagtagatgctatagataacatagtttgggaaaagaaatcattagtgacggaaaatcgtcttcac |
| | Sequence: | aaagaatgcttgttgagactatcgacagaggaacgtcatatatttttggattacaagaaatatggatcctctatccgactagaatta |
| | NC_006998.1) | gtcaatcttattcaagcaaaaacaaaaaactttacgatagactttaagctaaaatattttctaggatccggtgcccagtctaaaagt |
| | | tctttattacacgctattaatcatccaaagtcaaggcctaatacatctctggaaatagaatttacacctagagacaatgaaacagtt |
| | | ccatatgatgaactaataaaggaattgacgactctctcgcgtcatatatttatggcttctccagagaatgtaattctttctccgcctat |
| | | taacgcgcctataaaaacctttatgttgcctaaacaagatatagtaggtttggatctggaaaatctatatgccgtaactaagactg |
| | | acggcattcctataactatcagagttacatcaaacgggttgtattgttattttacacatcttggttatattattagatatcctgttaaga |
| | | gaataatagattccgaagtagtagtctttggtgaggcagttaaggataagaactggaccgtatatctcattaagctaatagagcc |
| | | tgtgaatgcaatcaatgatagactagaagaaagtaagtatgttgaatctaaactagtggatatttgtgatcggatagtattcaagtc |
| | | aaagaaatacgaaggtccgtttactacaactagtgaagtcgtcgatatgttatctacatatttaccaaagcaaccagaaggtgtta |
| | | ttctgttctattcaaagggacctaaatctaacattgattttaaaattaaaaaggaaaatactatagaccaaactgcaaatgtagtattt |
| | | aggtacatgtccagtgaaccaattatctttggagagtcgtctatctttgtagagtataagaaatttagcaacgataaaggctttcct |
| | | aaagaatatggttctggtaagattgtgttatataacggcgttaattatctaaataatatctattgtttggaatatattaatacacataat |
| | | gaagtgggtattaagtccgtggttgtacctattaagtttatagcagaattcttagttaatggagaaatacttaaacctagaattgata |
| | | aaaccatgaaatatattaactcagaagattattatggaaatcaacataatatcatagtcgaacatttaagagatcaaagcatcaaa |
| | | ataggagatatctttaacgaggataaactatcggatgtgggacatcaatacgccaataatgataaatttagattaaatccagaagt |
| | | tagttattttacgaataaacgaactagaggaccgttgggaattttatcaaactacgtcaagactcttcttatttctatgtattgttccaa |
| | | aacatttttagacgattccaacaaacgaaaggtattggcgattgattttggaaacggtgctgacctggaaaaatacttttatggag |
| | | agattgcgttattggtagcgacggatccggatgctgatgctatagctagaggaaatgaaagatacaacaaattaaactctggaa |
| | | ttaaaaccaagtactacaaatttgactacattcaggaaactattcgatccgatacatttgtctctagtgtcagagaagtattctatttt |
| | | ggaaagtttaatatcatcgactggcagtttgctatccattattcttttcatccgagacattatgctaccgtcatgaataacttatccga |
| | | actaactgcttctggaggcaaggtattaatcactaccatggacggagacaaattatcaaaattaacagataaaaagacttttataa |
| | | ttcataagaatttacctagtagcgaaaactatatgtctgtagaaaaaatagctgatgatagaatagtggtatataatccatcaacaa |
| | | tgtctactccaatgactgaatacattatcaaaaagaacgatatagtcagagtgtttaacgaatacggatttgttcttgtagataacgt |
| | | tgatttcgctacaattatagaacgaagtaaaaagtttattaatggcgcatctacaatggaagatagaccatctacaagaaacttttt |
| | | cgaactaaatagaggagccattaaatgtgaaggtttagatgtcgaagacttacttagttactatgttgtttatgtcttttctaagcggt |
| | | aa |
| 31 | D12 amino | MDEIVKNIREGTHVLLPFYETLPELNLSLGKSPLPSLEYGANYFLQISRVNDLNR |
| | acid sequence | MPTDMLKLFTHDIMLPESDLDKVYEILKINSVKYYGRSTKADAVVADLSARNK |
| | (Uniprot | LFKRERDAIKSNNHLTENNLYISDYKMLTFDVFRPLFDFVNEKYCIIKLPTLFGR |
| | Accession No. | GVIDTMRIYCSLFKNVRLLKCVSDSWLKDSAIMVASDVCKKNLDLFMSHVKSV |
| | P04318) | TKSSSWKDVNSVQFSILNNPVDTEFINKFLEFSNRVYEALYYVHSLLYSSMTSD |
| | | SKSIENKHQRRLVKLLL |
| 32 | D12 | atggatgaaattgtaaaaaatatccgggagggaacgcatgtccttcttccattttatgaaacattgccagaacttaatctgtctcta |
| | nucleotide | ggtaaaagcccattacctagtctggaatacggagctaattactttcttcagatttctagagttaatgatctaaatagaatgccgacc |
| | sequence | gacatgttaaaactttttacacatgatatcatgttaccagaaagcgatctagataaagtctatgaaattttaaagattaatagcgtaa |
| | (NCBI | agtattatgggaggagtactaaagcggacgccgtagttgccgacctcagcgcacgcaataaactgttcaaacgtgaacgaga |
| | Reference | tgctattaaatctaataatcatctcactgaaaacaatctatacattagcgattataagatgttaaccttcgacgtgtttcgaccattatt |
| | Sequence: | tgattttgtaaacgaaaaatattgtattattaaacttccaactttattcggtagaggtgtaatcgatactatgagaatatattgtagtct |
| | NC_006998.1) | ctttaaaaatgttagactgctaaaatgcgtaagcgatagctggttaaaagatagcgccattatggtggctagtgatgtttgtaaaa |
| | | aaaatttggatttatttatgtctcatgttaagtccgtcactaagtcttcttcttggaaggatgtgaacagtgttcaatttagtattttaaa |
| | | caatccagtggatacggaattcattaataagttcttagagttttcgaatagagtatacgaagctctctattacgttcactcgttgcttt |
| | | attctagtatgacttctgattcaaaaagtatcgaaaacaaacatcagagaagactagttaaactactgctgtga |
| 33 | D1 E. coli | gacgctaatgtcgtgtcttcttctaccatcgcaacctatattgacgctctggcaaaaaacgcctcggaactggaacaacgctcaa |
| | recode 1 | ccgcgtatgaaatcaacaatgaactggaactggtgtttatcaaaccgccgctgattacgctgaccaacgtggttaatatcagca |
| | (without tag) | ccattcaggaatcttttattcgtttcacggttaccaacaaagaaggcgtcaaaatccgcacgaaaattccgctgagcaaagttca |
| | | tggtctggatgtgaaaaacgttcaactggtcgacgcaatcgataatattgtgtgggaaaagaaaagcctggttaccgaaaatcg |
| | | tctgcataaagaatgcctgctgcgtctgagcacggaagaacgccacatctttctggactataaaaaatacggcagctctatccg |
| | | cctggaactggtgaacctgatccaggctaaaaccaaaaacttcacgatcgatttcaaactgaaatattttctgggcagtggtgct |
| | | caatccaaaagttccctgctgcatgcgatcaaccacccgaaaagtgtccgaatacctccctggaaattgaattcaccccgcg |
| | | cgacaacgaaacggtgccgtacgatgaactgattaaagaactgaccacgctgtcacgtcatatctttatggcgtcgccggaaa |
| | | acgttattctgagcccgccgatcaatgccccgattaaaaccttcatgctgccgaaacaggacattgttggcctggatctggaaa |
| | | acctgtatgcggtcacgaaaaccgatggtattccgatcaccattcgcgtgacgtcgaatggcctgtattgctactttacccacct |
| | | gggttatattatccgttacccggttaaacgcattatcgactccgaagtcgtggttttcggcgaagcggtcaaagataaaaattgg |
| | | accgtgtatctgatcaaactgattgaaccggtgaacgccatcaacgatcgtctggaagaatcaaaatacgtggaatcgaaact |
| | | ggttgacatctgtgatcgcatcgttttcaaaagcaaaaaatacgaaggtccgttcaccacgacctctgaagtcgtggatatgctg |
| | | agtacctatctgccgaaacagccggaaggcgtgatcctgttttacagcaaaggtccgaaatctaacatcgacttcaaaatcaaa |
| | | aaagaaaacaccatcgatcaaacggccaatgttgtctttcgttatatgtcateggaaccgattatctttggcgaaagctctatcttc |
| | | gtggaatacaaaaaattctcgaacgataaaggcttcccgaaagaatacggcagcggtaaaattgtcctgtataacggtgtgaat |
| | | tacctgaacaatatctattgcctggaatacattaacacccataatgaagttggcattaaatctgtggttgtcccgatcaaatttattg |
| | | cagaattcctggtcaacggtgaaatcctgaaaccgcgtattgacaaaaccatgaaatacatcaacagtgaagattactacggta |
| | | accagcataacatcatcgtggaacacctgcgcgaccaatctatcaaaatcggcgatatcttcaacgaagacaaactgagtgat |
| | | gtcggtcaccagtatgcgaacaatgataaatttcgtctgaacceggaagtgtcctacttcaccaataaacgtacgegeggccc |
| | | gctgggtatcctgtcaaattatgtcaaaaccctgctgatttcaatgtactgttcgaaaacgtttctggatgacagcaacaaacgca |
| | | aagttctggccattgactttggcaatggtgcagatctggaaaaatatttctacggcgaaatcgctctgctggttgegacegatcc |
| | | ggacgcggatgccattgcacgtggcaacgaacgctataacaaactgaattctggtatcaaaaccaaatactacaaattcgact |
| | | acatccaggaaaccattcgtagtgatacgttcgtgagttccgttcgcgaagtcttttatttcggcaaattcaacatcatcgattggc |
| | | aattcgccatccattattctttccatccgcgtcactacgcaaccgtgatgaacaatctgagtgaactgacggcttccggcggtaa |
| | | agttctgattacgacgatggatggtgataaactgtccaaactgaccgataagaaaaccttcattatccacaaaaacctgccgtca |
| | | tcggaaaactacatgtcagtggaaaaaatcgccgatgaccgcattgtggtttataacccgagcacgatgtctaccccgatgac |
| | | ggaatacatcattaagaaaaacgatatcgtccgtgtgtttaatgaatacggtttcgttctggtcgacaacgttgattttgcaaccatt |
| | | atcgaacgcagcaaaaaattcatcaatggcgcttccacgatggaagatcgtccgtcaacgcgcaactttttcgaactgaatcgc |
| | | ggtgcaattaaatgtgaaggtctggatgtggaagatctgctgtcctattatgtcgtgtatgtgttctctaaacgc |
| 34 | D1 E. coli | gacgccaacgtagtgagctcgtccacgattgctacatacatcgacgcactggctaaaaacgcgagtgaattagagcaacgttc |
| | recode 12 | aaccgcctatgaaatcaacaacgaacttgagctcgtctttattaagcctccgctaatcaccctgactaacgttgttaatatatctac |
| | (without tag) | catccaggaaagcttcattcgcttcactgttactaacaaagaaggcgtaaaaatcaggactaaaatcccattgtctaaggtgcac |
| | | gggctggatgtgaaaaacgttcagctggttgacgctattgacaacatcgtatgggaaaagaaatccctegtaaccgaaaaccg |
| | | tctgcataaagaatgtctgctgcgtctgagcacggaggaacgacacatctttctggattacaaaaaatatggtagttctattcgtc |
| | | tggagctggtgaacctgatccaggcaaagaccaaaaatttcacaattgacttcaaactaaaatactttctgggctccggtgcgc |
| | | agagcaaatcttccctgttgcatgctatcaaccacccgaaaagccgcccgaatacttctctggaaategagttcaccccccgcg |
| | | ataacgaaactgtcccatacgatgagcttattaaggaactgaccacgctgtcccgtcacatttttatggcgagcccggaaaacg |
| | | ttatattategccgcctatcaacgctccgatcaagaccttcatgttgccgaaacaagacatcgtcggtctggatctggagaacct |
| | | gtacgcagttactaaaaccgacggcatccccatcactatcagagtaacgtcaaacggattgtattgctatttcacccatctgggtt |
| | | acattattcgttacccggtgaaacgcatcatagattctgaagttgttgttttcggcgaagccgtaaaggacaaaaactggaccgt |
| | | ctatctgatcaagctaatcgaaccggttaatgctatcaacgatcggctggaagaatcgaaatacgtagaatctaaactggtggat |
| | | atttgcgaccgtattgtctttaaatcgaaaaagtacgagggtcctttcactactactagcgaagtcgtggacatgctctctacgtac |
| | | ctgccgaaacagcctgagggcgttatcctgttctatagcaaaggtccgaaatccaacatcgattttaagattaaaaaggaaaac |
| | | accattgatcagacggctaatgtagttttccggtacatgtctagcgagccgatcatctttggcgaatcttctatctttgtagaatata |
| | | aaaagttcagcaacgacaaaggattcccaaaagaatacgggtccgggaaaatcgtcttatacaacggtgttaactacttgaac |
| | | aacatctattgcctggaatatatcaatactcacaatgaagttggtattaaatcagtggttgttccgataaaattcatcgcggaatttc |
| | | tggtcaatggcgaaatcctgaaaccccgcattgataagaccatgaaatacataaactccgaagactactacggtaaccagcat |
| | | aacatcatcgtggaacacctgagagatcagagtatcaaaatcggcgacattttcaatgaggacaagttaagcgacgtgggcc |
| | | atcaatacgcaaacaacgacaaattccgtctgaacccggaggtttcctatttcaccaacaaacgtacccgaggtccgcttggca |
| | | tcctctccaattacgtaaaaaccctgctgatttctatgtattgttcaaaaacgttcctggatgacagcaacaaaaggaaggtactg |
| | | gctatcgatttcggtaacggcgcggatctggaaaagtacttttacggtgaaatcgctctgttagtcgcaactgatccggacgcc |
| | | gacgcaattgctcgcggaaatgaacgttacaacaaactgaactccggtattaaaacaaagtattataaattcgactatatccagg |
| | | agactatccgctctgatactttcgtgagcagcgtgcgtgaggttttttactttggtaaattcaacattattgactggcagtttgcgat |
| | | ccactacagctttcacccgcgtcactatgcgaccgttatgaataacctatcggaactcacggctagcggcggcaaagtgctga |
| | | ttactactatggacggtgacaaactgtctaagctgaccgataagaaaaccttcatcatccacaaaaacttgccaagttctgagaa |
| | | ctatatgtctgttgaaaaaattgeggacgaccgcatcgtcgtttacaacccatctaccatgtccacccctatgacagagtacatca |
| | | tcaaaaagaacgacatagttcgtgttttcaacgaatacggcttcgtactggtagataacgtcgattttgctaccattatcgagegtt |
| | | cgaaaaaattcattaacggtgcttccactatggaagatcgtccgtccactcgtaacttttttgaattaaaccgtggcgcaatcaaat |
| | | gcgaagggctggatgtggaagacctcctgtcttactacgttgtatacgtcttctctaaacgc |
| 35 | D12 E. coli | gatgaaatcgtcaaaaatatccgcgaaggcacgcacgtcctgctgccgttctatgaaaccctgccggaactgaatctgtcact |
| | recode 1 | gggcaaatctccgctgccgagtctggaatatggtgcaaactactttctgcagatttctcgtgtgaacgatctgaatcgcatgccg |
| | (without tag) | accgacatgctgaaactgttcacgcatgatatcatgctgccggaaagcgatctggacaaagtctacgaaatcctgaaaatcaa |
| | | ctccgttaaatactacggccgttcaaccaaagcggatgccgtggttgcagacctgtccgctcgcaataaactgtttaaacgtga |
| | | acgcgatgctattaaatcgaacaatcacctgaccgaaaacaacctgtacatcagcgattacaaaatgctgacgtttgacgtgttc |
| | | cgtccgctgttcgatttcgttaacgaaaaatactgcatcatcaaactgccgaccctgtttggccgtggtgtgattgatacgatgcg |
| | | catctactgcagcctgttcaaaaatgtccgcctgctgaaatgtgtgtcggatagctggctgaaagactctgcgattatggtggcc |
| | | agtgacgtttgtaagaaaaacctggacctgtttatgtcccatgtcaaatcagtgaccaaaagctctagttggaaagacgttaattc |
| | | ggtccaatttagcattctgaacaatccggttgatacggaattcatcaacaaattcctggaattctctaaccgtgtttacgaagcact |
| | | gtattacgtccacagtctgctgtactcctcaatgaccteggactccaaatccatcgaaaataaacatcaacgccgcctggtgaa |
| | | actgctgctg |
| 36 | D12 E. coli | Gatgagatcgttaagaacattcgtgaaggtacgcatgtgcttttgccattttacgaaactctcccggaactgaatctgtccttagg |
| | recode 2 | caaaagccctctaccctctctggagtatggggccaactacttcctgcaaatctcacgcgtcaacgacctgaatcgaatgccga |
| | (without tag) | ccgacatgctgaaactgttcactcacgatataatgctgccggaaagtgatctggacaaagtatatgaaatcctgaaaatcaaca |
| | | gcgttaagtactacggacggtcgaccaaagcggacgctgttgtagcagatctgtctgctcgcaacaaactctttaaacgtgaac |
| | | gtgacgctattaagtccaacaaccacctgacagagaacaatctctatatctctgactacaaaatgttgactttcgatgtgttccgtc |
| | | cgctgtttgatttcgtgaacgaaaaatattgcattatcaaactgccgaccctgttcggccgtggtgttattgacaccatgcgcatct |
| | | actgtagcctcttcaagaatgtcagactactgaaatgcgtgtccgatagctggctgaaagacagcgcaatcatggtagcctcag |
| | | acgtttgcaaaaagaacctggatctgtttatgtcccatgttaaatccgttactaagtctagctcgtggaaagatgttaacagcgtac |
| | | agttttctattttgaacaaccctgttgacacggaatttatcaacaaattcctggagttctctaaccgtgtatacgaagcgctgtatta |
| | | cgtgcactccttactgtactcttctatgaccagcgatagtaagtctatcgaaaataaacaccagegccgtctggtaaaactgctc |
| | | ctt |
| 37 | BCDRBS_alt1_ | gcgaaaaatcaataaggaggcaacaagatgtgcgaaaaacatcttaatcatgcaggggagggtttcta |
| | BD5 | |
| 38 | BCDRBS_alt1_ | gcgaaaaatcaataaggaggcaacaagatgtgcgaaaaacatcttaatcatgcatcggaccgtttcta |
| | BD8 | |
| 39 | FtsZ amino | MFEPMELTNDAVIKVIGVGGGGGNAVEHMVRERIEGVEFFAVNTDAQALRKT |
| | acid (E. coli) | AVGQTIQIGSGITKGLGAGANPEVGRNAADEDRDALRAALEGADMVFIAAGM |
| | | GGGTGTGAAPVVAEVAKDLGILTVAVVTKPFNFEGKKRMAFAEQGITELSKHV |
| | | DSLITIPNDKLLKVLGRGISLLDAFGAANDVLKGAVQGIAELITRPGLMNVDFA |
| | | DVRTVMSEMGYAMMGSGVASGEDRAEEAAEMAISSPLLEDIDLSGARGVLVN |
| | | ITAGFDLRLDEFETVGNTIRAFASDNATVVIGTSLDPDMNDELRVTVVATGIGM |
| | | DKRPEITLVTNKQVQQPVMDRYQQHGMAPLTQEQKPVAKVVNDNAPQTAKE |
| | | PDYLDIPAFLRKQAD |
| 40 | metK amino | MAKHLFTSESVSEGHPDKIADQISDAVLDAILEQDPKARVACETYVKTGMVLV |
| | acid (E. coli) | GGEITTSAWVDIEEITRNTVREIGYVHSDMGFDANSCAVLSAIGKQSPDINQGV |
| | | DRADPLEQGAGDQGLMFGYATNETDVLMPAPITYAHRLVQRQAEVRKNGTLP |
| | | WLRPDAKSQVTFQYDDGKIVGIDAVVLSTQHSEEIDQKSLQEAVMEEIIKPILPA |
| | | EWLTSATKFFINPTGRFVIGGPMGDCGLTGRKIIVDTYGGMARHGGGAFSGKD |
| | | PSKVDRSAAYAARYVAKNIVAAGLADRCEIQVSYAIGVAEPTSIMVETFGTEKV |
| | | PSEQLTLLVREFFDLRPYGLIQMLDLLHPIYKETAAYGHFGREHFPWEKTDKAQ |
| | | LLRDAAGLK |
| 41 | mreB amino | MLKKFRGMFSNDLSIDLGTANTLIYVKGQGIVLNEPSVVAIRQDRAGSPKSVAA |
| | acid (E. coli) | VGHDAKQMLGRTPGNIAAIRPMKDGVIADFFVTEKMLQHFIKQVHSNSFMRPS |
| | | PRVLVCVPVGATQVERRAIRESAQGAGAREVFLIEEPMAAAIGAGLPVSEATGS |
| | | MVVDIGGGTTEVAVISLNGVVYSSSVRIGGDRFDEAIINYVRRNYGSLIGEATAE |
| | | RIKHEIGSAYPGDEVREIEVRGRNLAEGVPRGFTLNSNEILEALQEPLTGIVSAV |
| | | MVALEQCPPELASDISERGMVLTGGGALLRNLDRLLMEETGIPVVVAEDPLTC |
| | | VARGGGKALEMIDMHGGDLFSEE |
| 42 | FtsZ nucleic | atgtttgaaccaatggaacttaccaatgacgcggtgattaaagtcatcggcgtcggcggcggcggcggtaatgctgttgaaca |
| | acid (E. coli) | catggtgcgcgagcgcattgaaggtgttgaattcttcgcggtaaataccgatgcacaagcgctgcgtaaaacagcggttggac |
| | | agacgattcaaatcggtagcggtatcaccaaaggactgggcgctggcgctaatccagaagttggccgcaatgcggctgatg |
| | | aggatcgcgatgcattgcgtgcggcgctggaaggtgcagacatggtctttattgctgcgggtatgggtggtggtaccggtaca |
| | | ggtgcagcaccagtcgtcgctgaagtggcaaaagatttgggtatcctgaccgttgctgtcgtcactaagcctttcaactttgaag |
| | | gcaagaagcgtatggcattcgcggagcaggggatcactgaactgtccaagcatgtggactctctgatcactatcccgaacga |
| | | caaactgctgaaagttctgggccgcggtatctccctgctggatgcgtttggcgcagcgaacgatgtactgaaaggcgctgtgc |
| | | aaggtatcgctgaactgattactcgtccgggtttgatgaacgtggactttgcagacgtacgcaccgtaatgtctgagatgggcta |
| | | cgcaatgatgggttctggcgtggcgagcggtgaagaccgtgcggaagaagctgctgaaatggctatctcttctccgctgctg |
| | | gaagatatcgacctgtctggcgcgcgcggcgtgctggttaacatcacggcgggcttcgacctgcgtctggatgagttcgaaa |
| | | cggtaggtaacaccatccgtgcatttgcttccgacaacgcgactgtggttatcggtacttctcttgacccggatatgaatgacga |
| | | gctgcgcgtaaccgttgttgcgacaggtatcggcatggacaaacgtcctgaaatcactctggtgaccaataagcaggttcagc |
| | | agccagtgatggatcgctaccagcagcatgggatggctccgctgacccaggagcagaagccggttgctaaagtcgtgaatg |
| | | acaatgcgccgcaaactgcgaaagagccggattatctggatatcccagcattcctgcgtaagcaagctgattaa |
| 43 | metK nucleic | atggcaaaacacctttttacgtccgagtccgtctctgaagggcatcctgacaaaattgctgaccaaatttctgatgccgttttaga |
| | acid (E. coli) | cgcgatcctcgaacaggatccgaaagcacgcgttgcttgcgaaacctacgtaaaaaccggcatggttttagttggcggcgaa |
| | | atcaccaccagcgcctgggtagacatcgaagagatcacccgtaacaccgttcgcgaaattggctatgtgcattccgacatgg |
| | | gctttgacgctaactcctgtgcggttctgagcgctatcggcaaacagtctcctgacatcaaccagggcgttgaccgtgccgatc |
| | | cgctggaacagggcgcgggtgaccagggtctgatgtttggctacgcaactaatgaaaccgacgtgctgatgccagcacctat |
| | | cacctatgcacaccgtctggtacagcgtcaggctgaagtgcgtaaaaacggcactctgccgtggctgcgcccggacgcgaa |
| | | aagccaggtgacttttcagtatgacgacggcaaaatcgttggtatcgatgctgtcgtgctttccactcagcactctgaagagatc |
| | | gaccagaaatcgctgcaagaagcggtaatggaagagatcatcaagccaattctgcccgctgaatggctgacttctgccacca |
| | | aattcttcatcaacccgaccggtcgtttcgttatcggtggcccaatgggtgactgcggtctgactggtcgtaaaattatcgttgat |
| | | acctacggcggcatggcgcgtcacggtggcggtgcattctctggtaaagatccatcaaaagtggaccgttccgcagcctacg |
| | | cagcacgttatgtcgcgaaaaacatcgttgctgctggcctggccgatcgttgtgaaattcaggtttcctacgcaatcggcgtgg |
| | | ctgaaccgacctccatcatggtagaaactttcggtactgagaaagtgccttctgaacaactgaccctgctggtacgtgagttctt |
| | | cgacctgcgcccatacggtctgattcagatgctggatctgctgcacccgatctacaaagaaaccgcagcatacggtcactttg |
| | | gtcgtgaacatttcccgtgggaaaaaaccgacaaagcgcagctgctgcgcgatgctgccggtctgaagtaa |
| 44 | mreB nucleic | ttactcttcgctgaacaggtcgccgccgtgcatgtcgatcatttccagcgctttgccgccaccgcgcgccacacaggtcagcg |
| | acid (E. coli) | ggtcttcagcaacaacgactggaatgccggtttcttccattaacaaacggtcaaggttacgcagcagtgcgccaccaccggtg |
| | | agcaccatgccgcgctcggagatgtcggaagccagttccggcgggcactgttccagtgcaaccattacegcgctcacaatac |
| | | cggtcagcggttcctgcagtgcttcgaggatttcattggagttcagggtaaaaccgcgtggaacaccttctgccaggttacggc |
| | | cacgaacttcgatttcacggacttcatcgcccggataagccgaaccgatttcgtgcttgatacgttctgcggtggcttcaccgat |
| | | cagagaaccgtaattacgacgcacatagttgatgatagcttcgtcgaaacggtcaccaccaatgcgcacagaagaggagtaa |
| | | accacaccgttcaaggagataacagcaacttcagtggtaccaccaccgatatcaaccaccatagaaccggtcgcttcagaaa |
| | | ccggcaggccagcaccaattgcggcagccateggttcttcaatcaggaagacttcacgggcaccagcgccctgcgcggatt |
| | | cacgaattgcgcggcgttcaacctgggtcgcgccaaccggcacacaaaccagaacgcgcgggcttggacgcataaagctg |
| | | ttgctgtgcacttgtttgatgaagtgctggagcattttttcagtcacgaagaagtcggcgataacgccgtctttcattgggcgaat |
| | | ggcagcaatattgcccggcgtacggcccagcatctgcttcgcgtcatgacctactgcagctacgcttttcggtgaaccggcac |
| | | gatcctgacgaatggccaccacggaaggctcattcaatacgatgccttgtccttttacataaatgagggtattcgcagtacccag |
| | | gtcaatggacaagtcattggaaaacatgccacgaaattttttcaacat |
| 45 | BCDRBS_alt1_ | gcgaaaaatcaataaggaggcaacaagatgtgcgaaaaacatcttaatcatgcgagggatggtttctaatg |
| | BD21 | |
| 46 | apFAB69 | ttgacatcgcatctttttgtaccatacttacagccattgtac |
| 47 | apFAB124 | tcgacatttatcccttgcggcgaatacttacagcca |
| 48 | apFAB277 | ttccctattaatcatccggctcgtataatgtgtgga |
| 21 | Combination | aattgtgagcggataacaattacgagcttcatgcacagtgaaatcatgaaaaatttatttgctttgtgagcggataacaattataat |
| | of genetic | atgtggaattgtgagcgctcacaattccacagcgaaaaatcaataaggaggcaacaagatgtgcgaaaaacatcttaatcatg |
| | elements | cacaggagactttctaatgaaacatcaccatcaccatcaccccatgagegattacgacatccccactactgagaatctttattttc |
| | expressed in | agggcgccgacgctaatgtcgtgtcttcttctaccatcgcaacctatattgacgctctggcaaaaaacgcctcggaactggaac |
| | strain 870868 | aacgctcaaccgcgtatgaaatcaacaatgaactggaactggtgtttatcaaaccgccgctgattacgctgaccaacgtggtta |
| | (Promoter | atatcagcaccattcaggaatcttttattcgtttcacggttaccaacaaagaaggcgtcaaaatccgcacgaaaattccgctgag |
| | (P(T5) | caaagttcatggtctggatgtgaaaaacgttcaactggtcgacgcaatcgataatattgtgtgggaaaagaaaagcctggttac |
| | 2xlacO); RBS | cgaaaatcgtctgcataaagaatgcctgctgcgtctgagcacggaagaacgccacatctttctggactataaaaaatacggca |
| | (BCDRBS_alt1_ | gctctatccgcctggaactggtgaacctgatccaggctaaaaccaaaaacttcacgatcgatttcaaactgaaatattttctggg |
| | BD1); His- | cagtggtgctcaatccaaaagttccctgctgcatgcgatcaaccacccgaaaagtcgtccgaatacctccctggaaattgaatt |
| | Tag; D1 (E. | caccccgcgcgacaacgaaacggtgccgtacgatgaactgattaaagaactgaccacgctgtcacgtcatatctttatggcgt |
| | coli recode 1); | cgccggaaaacgttattctgagcccgccgatcaatgccccgattaaaaccttcatgctgccgaaacaggacattgttggcctg |
| | Terminator | gatctggaaaacctgtatgcggtcacgaaaaccgatggtattccgatcaccattcgcgtgacgtcgaatggcctgtattgctact |
| | (Bba_J61048); | ttacccacctgggttatattatccgttacccggttaaacgcattatcgactccgaagtcgtggttttcggcgaagcggtcaaagat |
| | Promoter | aaaaattggaccgtgtatctgatcaaactgattgaaccggtgaacgccatcaacgatcgtctggaagaatcaaaatacgtgga |
| | (Ptac); RBS | atcgaaactggttgacatctgtgatcgcatcgttttcaaaagcaaaaaatacgaaggtccgttcaccacgacctctgaagtcgtg |
| | (BCDRBS_alt1_ | gatatgctgagtacctatctgccgaaacagccggaaggcgtgatcctgttttacagcaaaggtccgaaatctaacatcgacttc |
| | BD6); D12 | aaaatcaaaaaagaaaacaccatcgatcaaacggccaatgttgtctttcgttatatgtcatcggaaccgattatctttggcgaaa |
| | (E. coli recode | gctctatcttcgtggaatacaaaaaattctcgaacgataaaggcttcccgaaagaatacggcagcggtaaaattgtcctgtataa |
| | 1); Twin Strep | cggtgtgaattacctgaacaatatctattgcctggaatacattaacacccataatgaagttggcattaaatctgtggttgtcccgat |
| | Tag | caaatttattgcagaattcctggtcaacggtgaaatcctgaaaccgcgtattgacaaaaccatgaaatacatcaacagtgaagat |
| | Terminator | tactacggtaaccagcataacatcatcgtggaacacctgegcgaccaatctatcaaaatcggcgatatcttcaacgaagacaa |
| | ((BBa_B0015 | actgagtgatgtcggtcaccagtatgcgaacaatgataaatttcgtctgaacccggaagtgtcctacttcaccaataaacgtacg |
| | (Double | cgcggcccgctgggtatcctgtcaaattatgtcaaaaccctgctgatttcaatgtactgttcgaaaacgtttctggatgacagcaa |
| | Terminator | caaacgcaaagttctggccattgactttggcaatggtgcagatctggaaaaatatttctacggcgaaatcgctctgctggttgcg |
| | B0010, | accgatccggacgcggatgccattgcacgtggcaacgaacgctataacaaactgaattctggtatcaaaaccaaatactacaa |
| | B0012)); | attcgactacatccaggaaaccattcgtagtgatacgttcgtgagttccgttcgcgaagtcttttatttcggcaaattcaacatcatc |
| | Terminator | gattggcaattcgccatccattattctttccatccgcgtcactacgcaaccgtgatgaacaatctgagtgaactgacggcttccg |
| | (T7 | gcggtaaagttctgattacgacgatggatggtgataaactgtccaaactgaccgataagaaaaccttcattatccacaaaaacct |
| | terminator) | gccgtcatcggaaaactacatgtcagtggaaaaaatcgccgatgaccgcattgtggtttataacccgagcacgatgtctaccc |
| | | cgatgacggaatacatcattaagaaaaacgatatcgtccgtgtgtttaatgaatacggtttcgttctggtcgacaacgttgattttg |
| | | caaccattatcgaacgcagcaaaaaattcatcaatggcgcttccacgatggaagatcgtccgtcaacgcgcaactttttcgaac |
| | | tgaatcgcggtgcaattaaatgtgaaggtctggatgtggaagatctgctgtcctattatgtcgtgtatgtgttctctaaacgctaac |
| | | cggcttatcggtcagtttcacctgatttacgtaaaaacccgcttcggcgggtttttgcttttggaggggcagaaagatgaatgact |
| | | gtccacgacgctatacccaaaagaaatgttgacaattaatcatcggctcgtataatgtgtggaattgtgagcgctcacaattgcg |
| | | aaaaatcaataaggaggcaacaagatgtgcgaaaaacatcttaatcatgcgccggaggttttctaatggatgaaatcgtcaaaa |
| | | atatccgcgaaggcacgcacgtcctgctgccgttctatgaaaccctgccggaactgaatctgtcactgggcaaatctccgctg |
| | | ccgagtctggaatatggtgcaaactactttctgcagatttctcgtgtgaacgatctgaatcgcatgccgaccgacatgctgaaac |
| | | tgttcacgcatgatatcatgctgccggaaagcgatctggacaaagtctacgaaatcctgaaaatcaactccgttaaatactacg |
| | | gccgttcaaccaaagcggatgccgtggttgcagacctgtccgctcgcaataaactgtttaaacgtgaacgcgatgctattaaat |
| | | cgaacaatcacctgaccgaaaacaacctgtacatcagcgattacaaaatgctgacgtttgacgtgttccgtccgctgttcgattt |
| | | cgttaacgaaaaatactgcatcatcaaactgccgaccctgtttggccgtggtgtgattgatacgatgcgcatctactgcagcctg |
| | | ttcaaaaatgtccgcctgctgaaatgtgtgtcggatagctggctgaaagactctgcgattatggtggccagtgacgtttgtaaga |
| | | aaaacctggacctgtttatgtcccatgtcaaatcagtgaccaaaagctctagttggaaagacgttaattcggtccaatttagcatt |
| | | ctgaacaatccggttgatacggaattcatcaacaaattcctggaattctctaaccgtgtttacgaagcactgtattacgtccacagt |
| | | ctgctgtactcctcaatgaccteggactccaaatccatcgaaaataaacatcaacgccgcctggtgaaactgctgctggggag |
| | | cgcttggagccacccgcagttcgaaaaaggtggaggttctggcggtggatcgggaggttcagcgtggagccacccgcagtt |
| | | cgagaaataaccaggcatcaaataaaacgaaaggctcagtcgaaagactgggcctttcgttttatctgttgtttgtcggtgaacg |
| | | ctctctactagagtcacactggctcaccttcggggggcctttctgcgtttataataaccccttggggcctctaaacgggtcttga |
| | | ggggttttttgc |
| 49 | Combination | tcgacatttatcccttgcggcgaatacttacagccagcgaaaaatcaataaggaggcaacaagatgtgcgaaaaacatcttaat |
| | of genetic | catgcggtggagggtttctaatgaaacatcaccatcaccatcaccccatgagegattacgacatccccactactgagaatcttta |
| | elements | ttttcagggcgccgacgccaacgtagtgagctcgtccacgattgctacatacatcgacgcactggctaaaaacgcgagtgaat |
| | expressed in | tagagcaacgttcaaccgcctatgaaatcaacaacgaacttgagctcgtctttattaagcctccgctaatcaccctgactaacgtt |
| | strain 807175 | gttaatatatctaccatccaggaaagcttcattcgcttcactgttactaacaaagaaggcgtaaaaatcaggactaaaatcccatt |
| | (Promoter | gtctaaggtgcacgggctggatgtgaaaaacgttcagctggttgacgctattgacaacatcgtatgggaaaagaaatccctcgt |
| | (apFAB124); | aaccgaaaaccgtctgcataaagaatgtctgctgcgtctgagcacggaggaacgacacatctttctggattacaaaaaatatg |
| | RBS | gtagttctattcgtctggagctggtgaacctgatccaggcaaagaccaaaaatttcacaattgacttcaaactaaaatactttctg |
| | (BCDRBS_alt1_ | ggctccggtgcgcagagcaaatcttccctgttgcatgctatcaaccacccgaaaagccgcccgaatacttctctggaaatoga |
| | BD14); His- | gttcaccccccgcgataacgaaactgtcccatacgatgagcttattaaggaactgaccacgctgtcccgtcacatttttatggcg |
| | Tag; D1 (E. | agcccggaaaacgttatattategccgcctatcaacgctccgatcaagaccttcatgttgccgaaacaagacatcgtcggtctg |
| | coli recode | gatctggagaacctgtacgcagttactaaaaccgacggcatccccatcactatcagagtaacgtcaaacggattgtattgctatt |
| | 12); RBS | tcacccatctgggttacattattcgttacccggtgaaacgcatcatagattctgaagttgttgttttcggcgaagccgtaaaggac |
| | (BCDRBS_alt1_ | aaaaactggaccgtctatctgatcaagctaatcgaaccggttaatgctatcaacgatcggctggaagaatcgaaatacgtagaa |
| | BD15); D12 | tctaaactggtggatatttgcgaccgtattgtctttaaatcgaaaaagtacgagggtcctttcactactactagcgaagtcgtgga |
| | (E. coli recode | catgctctctacgtacctgccgaaacagcctgagggcgttatcctgttctatagcaaaggtccgaaatccaacatcgattttaag |
| | 2); Twin Strep | attaaaaaggaaaacaccattgatcagacggctaatgtagttttccggtacatgtctagcgagccgatcatctttggcgaatcttc |
| | Tag; | tatctttgtagaatataaaaagttcagcaacgacaaaggattcccaaaagaatacgggtccgggaaaatcgtcttatacaacgg |
| | Terminator | tgttaactacttgaacaacatctattgcctggaatatatcaatactcacaatgaagttggtattaaatcagtggttgttccgataaaat |
| | ((BBa_B0015 | tcatcgcggaatttctggtcaatggcgaaatcctgaaaccccgcattgataagaccatgaaatacataaactccgaagactact |
| | (Double | acggtaaccagcataacatcatcgtggaacacctgagagatcagagtatcaaaatcggcgacattttcaatgaggacaagtta |
| | Terminator | agcgacgtgggccatcaatacgcaaacaacgacaaattccgtctgaacccggaggtttcctatttcaccaacaaacgtacccg |
| | B0010, | aggtccgcttggcatcctctccaattacgtaaaaaccctgctgatttctatgtattgttcaaaaacgttcctggatgacagcaacaa |
| | B0012)) | aaggaaggtactggctatcgatttcggtaacggcgcggatctggaaaagtacttttacggtgaaatcgctctgttagtcgcaact |
| | | gatccggacgccgacgcaattgctcgcggaaatgaacgttacaacaaactgaactccggtattaaaacaaagtattataaattc |
| | | gactatatccaggagactatccgctctgatactttcgtgagcagcgtgcgtgaggttttttactttggtaaattcaacattattgact |
| | | ggcagtttgcgatccactacagctttcacccgcgtcactatgcgaccgttatgaataacctatcggaactcacggctagcggcg |
| | | gcaaagtgctgattactactatggacggtgacaaactgtctaagctgaccgataagaaaaccttcatcatccacaaaaacttgc |
| | | caagttctgagaactatatgtctgttgaaaaaattgcggacgaccgcatcgtcgtttacaacccatctaccatgtccacccctatg |
| | | acagagtacatcatcaaaaagaacgacatagttcgtgttttcaacgaatacggcttcgtactggtagataacgtcgattttgctac |
| | | cattatcgagcgttcgaaaaaattcattaacggtgcttccactatggaagatcgtccgtccactcgtaacttttttgaattaaaccgt |
| | | ggcgcaatcaaatgcgaagggctggatgtggaagacctcctgtcttactacgttgtatacgtcttctctaaacgctaaaataattt |
| | | tgtttaactttaagaaggaggtatatccatggctagcatgactaaacatcttaatcatgcgggggagtctttctaatggatgagatc |
| | | gttaagaacattcgtgaaggtacgcatgtgcttttgccattttacgaaactctcccggaactgaatctgtccttaggcaaaagccc |
| | | tctaccctctctggagtatggggccaactacttcctgcaaatctcacgcgtcaacgacctgaatcgaatgccgaccgacatgct |
| | | gaaactgttcactcacgatataatgctgccggaaagtgatctggacaaagtatatgaaatcctgaaaatcaacagcgttaagta |
| | | ctacggacggtcgaccaaagcggacgctgttgtagcagatctgtctgctcgcaacaaactctttaaacgtgaacgtgacgctat |
| | | taagtccaacaaccacctgacagagaacaatctctatatctctgactacaaaatgttgactttcgatgtgttccgtccgctgtttga |
| | | tttcgtgaacgaaaaatattgcattatcaaactgccgaccctgttcggccgtggtgttattgacaccatgcgcatctactgtagcct |
| | | cttcaagaatgtcagactactgaaatgcgtgtccgatagctggctgaaagacagcgcaatcatggtagcctcagacgtttgcaa |
| | | aaagaacctggatctgtttatgtcccatgttaaatccgttactaagtctagctcgtggaaagatgttaacagcgtacagttttctattt |
| | | tgaacaaccctgttgacacggaatttatcaacaaattcctggagttctctaaccgtgtatacgaagcgctgtattacgtgcactcc |
| | | ttactgtactcttctatgaccagcgatagtaagtctatcgaaaataaacaccagcgccgtctggtaaaactgctccttgggagcg |
| | | cttggagccacccgcagttcgaaaaaggtggaggttctggcggtggatcgggaggttcagcgtggagccacccgcagttcg |
| | | agaaataaccaggcatcaaataaaacgaaaggctcagtcgaaagactgggcctttcgttttatctgttgtttgtcggtgaacgct |
| | | ctctactagagtcacactggctcaccttcgggtgggcctttctgcgtttata |
| 50 | Combination | ttgacatcgcatctttttgtaccatacttacagccattgtacgcgaaaaatcaataaggaggcaacaagatgtgcgaaaaacatc |
| | of genetic | ttaatcatgcggtggagggtttctaatgaaacatcaccatcaccatcaccccatgagegattacgacatccccactactgagaat |
| | elements | ctttattttcagggcgccgacgccaacgtagtgagctcgtccacgattgctacatacatcgacgcactggctaaaaacgcgagt |
| | expressed in | gaattagagcaacgttcaaccgcctatgaaatcaacaacgaacttgagctcgtctttattaagcctccgctaatcaccctgacta |
| | strain 807176 | acgttgttaatatatctaccatccaggaaagcttcattcgcttcactgttactaacaaagaaggcgtaaaaatcaggactaaaatc |
| | (Promoter | ccattgtctaaggtgcacgggctggatgtgaaaaacgttcagctggttgacgctattgacaacatcgtatgggaaaagaaatcc |
| | (apFAB69); | ctcgtaaccgaaaaccgtctgcataaagaatgtctgctgcgtctgagcacggaggaacgacacatctttctggattacaaaaaa |
| | RBS | tatggtagttctattcgtctggagctggtgaacctgatccaggcaaagaccaaaaatttcacaattgacttcaaactaaaatacttt |
| | (BCDRBS_alt1_ | ctgggctccggtgcgcagagcaaatcttccctgttgcatgctatcaaccacccgaaaagccgcccgaatacttctctggaaatc |
| | BD14); His- | gagttcaccccccgcgataacgaaactgtcccatacgatgagcttattaaggaactgaccacgctgtcccgtcacatttttatgg |
| | Tag; D1 (E. | cgagcccggaaaacgttatattatogccgcctatcaacgctccgatcaagaccttcatgttgccgaaacaagacategtcggtc |
| | coli recode | tggatctggagaacctgtacgcagttactaaaaccgacggcatccccatcactatcagagtaacgtcaaacggattgtattgct |
| | 12); RBS | atttcacccatctgggttacattattcgttacccggtgaaacgcatcatagattctgaagttgttgttttcggcgaagccgtaaagg |
| | (BCDRBS_alt1_ | acaaaaactggaccgtctatctgatcaagctaatcgaaccggttaatgctatcaacgatcggctggaagaatcgaaatacgtag |
| | BD21); D12 | aatctaaactggtggatatttgcgaccgtattgtctttaaatcgaaaaagtacgagggtcctttcactactactagcgaagtcgtg |
| | (E. coli recode | gacatgctctctacgtacctgccgaaacagcctgagggcgttatcctgttctatagcaaaggtccgaaatccaacatcgatttta |
| | 2); Twin Strep | agattaaaaaggaaaacaccattgatcagacggctaatgtagttttccggtacatgtctagcgagccgatcatctttggcgaatc |
| | Tag; | ttctatctttgtagaatataaaaagttcagcaacgacaaaggattcccaaaagaatacgggtccgggaaaategtcttatacaac |
| | Terminator | ggtgttaactacttgaacaacatctattgcctggaatatatcaatactcacaatgaagttggtattaaatcagtggttgttccgataa |
| | ((BBa_B0015 | aattcatcgcggaatttctggtcaatggcgaaatcctgaaaccccgcattgataagaccatgaaatacataaactccgaagact |
| | (Double | actacggtaaccagcataacatcatcgtggaacacctgagagatcagagtatcaaaatcggcgacattttcaatgaggacaag |
| | Terminator | ttaagcgacgtgggccatcaatacgcaaacaacgacaaattccgtctgaacccggaggtttcctatttcaccaacaaacgtacc |
| | B0010, | cgaggtccgcttggcatcctctccaattacgtaaaaaccctgctgatttctatgtattgttcaaaaacgttcctggatgacagcaa |
| | B0012)) | caaaaggaaggtactggctatcgatttcggtaacggcgcggatctggaaaagtacttttacggtgaaatcgctctgttagtcgc |
| | | aactgatccggacgccgacgcaattgctcgcggaaatgaacgttacaacaaactgaactccggtattaaaacaaagtattata |
| | | aattcgactatatccaggagactatccgctctgatactttcgtgagcagcgtgcgtgaggttttttactttggtaaattcaacattatt |
| | | gactggcagtttgcgatccactacagctttcacccgcgtcactatgcgaccgttatgaataacctatcggaactcacggctagc |
| | | ggcggcaaagtgctgattactactatggacggtgacaaactgtctaagctgaccgataagaaaaccttcatcatccacaaaaa |
| | | cttgccaagttctgagaactatatgtctgttgaaaaaattgeggacgaccgcategtcgtttacaacccatctaccatgtccaccc |
| | | ctatgacagagtacatcatcaaaaagaacgacatagttcgtgttttcaacgaatacggcttcgtactggtagataacgtcgatttt |
| | | gctaccattatcgagcgttcgaaaaaattcattaacggtgcttccactatggaagatcgtccgtccactcgtaacttttttgaattaa |
| | | accgtggcgcaatcaaatgcgaagggctggatgtggaagacctcctgtcttactacgttgtatacgtcttctctaaacgctaagc |
| | | gaaaaatcaataaggaggcaacaagatgtgcgaaaaacatcttaatcatgcgagggatggtttctaatggatgagatcgttaa |
| | | gaacattcgtgaaggtacgcatgtgcttttgccattttacgaaactctcccggaactgaatctgtccttaggcaaaagccctctac |
| | | cctctctggagtatggggccaactacttcctgcaaatctcacgcgtcaacgacctgaatcgaatgccgaccgacatgctgaaa |
| | | ctgttcactcacgatataatgctgccggaaagtgatctggacaaagtatatgaaatcctgaaaatcaacagcgttaagtactacg |
| | | gacggtcgaccaaagcggacgctgttgtagcagatctgtctgctcgcaacaaactctttaaacgtgaacgtgacgctattaagt |
| | | ccaacaaccacctgacagagaacaatctctatatctctgactacaaaatgttgactttcgatgtgttccgtccgctgtttgatttcgt |
| | | gaacgaaaaatattgcattatcaaactgccgaccctgttcggccgtggtgttattgacaccatgcgcatctactgtagcctcttca |
| | | agaatgtcagactactgaaatgcgtgtccgatagctggctgaaagacagcgcaatcatggtagcctcagacgtttgcaaaaag |
| | | aacctggatctgtttatgtcccatgttaaatccgttactaagtctagctcgtggaaagatgttaacagcgtacagttttctattttgaa |
| | | caaccctgttgacacggaatttatcaacaaattcctggagttctctaaccgtgtatacgaagcgctgtattacgtgcactccttact |
| | | gtactcttctatgaccagcgatagtaagtctatcgaaaataaacaccagcgccgtctggtaaaactgctccttgggagcgcttg |
| | | gagccacccgcagttcgaaaaaggtggaggttctggcggtggatcgggaggttcagcgtggagccacccgcagttcgaga |
| | | aataaccaggcatcaaataaaacgaaaggctcagtcgaaagactgggcctttcgttttatctgttgtttgtcggtgaacgctctct |
| | | actagagtcacactggctcaccttcggggggcctttctgcgtttata |
| 51 | Combination | tcgacatttatcccttgcggcgaatacttacagccagcgaaaaatcaataaggaggcaacaagatgtgcgaaaaacatcttaat |
| | of genetic | catgcggtggagggtttctaatgaaacatcaccatcaccatcaccccatgagegattacgacatccccactactgagaatcttta |
| | elements | ttttcagggcgccgacgctaatgtcgtgtcttcttctaccatcgcaacctatattgacgctctggcaaaaaacgcctcggaactg |
| | expressed in | gaacaacgctcaaccgcgtatgaaatcaacaatgaactggaactggtgtttatcaaaccgccgctgattacgctgaccaacgt |
| | strain 815930 | ggttaatatcagcaccattcaggaatcttttattcgtttcacggttaccaacaaagaaggcgtcaaaatccgcacgaaaattccg |
| | (Promoter | ctgagcaaagttcatggtctggatgtgaaaaacgttcaactggtcgacgcaatcgataatattgtgtgggaaaagaaaagcctg |
| | (apFAB124); | gttaccgaaaatcgtctgcataaagaatgcctgctgcgtctgagcacggaagaacgccacatctttctggactataaaaaatac |
| | RBS | ggcagctctatccgcctggaactggtgaacctgatccaggctaaaaccaaaaacttcacgatcgatttcaaactgaaatattttc |
| | (BCDRBS_alt1_ | tgggcagtggtgctcaatccaaaagttccctgctgcatgcgatcaaccacccgaaaagtegtccgaatacctccctggaaatt |
| | BD14); His- | gaattcaccccgcgcgacaacgaaacggtgccgtacgatgaactgattaaagaactgaccacgctgtcacgtcatatctttatg |
| | Tag; D1 (E. | gcgtcgccggaaaacgttattctgagcccgccgatcaatgccccgattaaaaccttcatgctgccgaaacaggacattgttgg |
| | coli recode 1); | cctggatctggaaaacctgtatgcggtcacgaaaaccgatggtattccgatcaccattcgcgtgacgtcgaatggcctgtattg |
| | RBS | ctactttacccacctgggttatattatccgttacccggttaaacgcattatcgactccgaagtcgtggttttcggcgaagcggtca |
| | (BCDRBS_alt1_ | aagataaaaattggaccgtgtatctgatcaaactgattgaaccggtgaacgccatcaacgatcgtctggaagaatcaaaatacg |
| | BD21); D12 | tggaatcgaaactggttgacatctgtgatcgcatcgttttcaaaagcaaaaaatacgaaggtccgttcaccacgacctctgaagt |
| | (E. coli recode | cgtggatatgctgagtacctatctgccgaaacagccggaaggcgtgatcctgttttacagcaaaggtccgaaatctaacatcga |
| | 1); Twin Strep | cttcaaaatcaaaaaagaaaacaccatcgatcaaacggccaatgttgtctttcgttatatgtcatcggaaccgattatctttggcg |
| | Tag; | aaagctctatcttcgtggaatacaaaaaattctcgaacgataaaggcttcccgaaagaatacggcageggtaaaattgtcctgt |
| | Terminator | ataacggtgtgaattacctgaacaatatctattgcctggaatacattaacacccataatgaagttggcattaaatctgtggttgtcc |
| | ((BBa_B0015 | cgatcaaatttattgcagaattcctggtcaacggtgaaatcctgaaaccgcgtattgacaaaaccatgaaatacatcaacagtga |
| | (Double | agattactacggtaaccagcataacatcatcgtggaacacctgcgcgaccaatctatcaaaatcggcgatatcttcaacgaaga |
| | Terminator | caaactgagtgatgtcggtcaccagtatgcgaacaatgataaatttcgtctgaacccggaagtgtcctacttcaccaataaacgt |
| | B0010, | acgcgcggcccgctgggtatcctgtcaaattatgtcaaaaccctgctgatttcaatgtactgttcgaaaacgtttctggatgacag |
| | B0012)) | caacaaacgcaaagttctggccattgactttggcaatggtgcagatctggaaaaatatttctacggcgaaatcgctctgctggtt |
| | | gcgaccgatccggacgcggatgccattgcacgtggcaacgaacgctataacaaactgaattctggtatcaaaaccaaatact |
| | | acaaattcgactacatccaggaaaccattcgtagtgatacgttcgtgagttccgttcgcgaagtcttttatttcggcaaattcaaca |
| | | tcatcgattggcaattcgccatccattattctttccatccgcgtcactacgcaaccgtgatgaacaatctgagtgaactgacggct |
| | | tccggcggtaaagttctgattacgacgatggatggtgataaactgtccaaactgaccgataagaaaaccttcattatccacaaa |
| | | aacctgccgtcatcggaaaactacatgtcagtggaaaaaatcgccgatgaccgcattgtggtttataacccgagcacgatgtct |
| | | accccgatgacggaatacatcattaagaaaaacgatatcgtccgtgtgtttaatgaatacggtttcgttctggtcgacaacgttga |
| | | ttttgcaaccattatcgaacgcagcaaaaaattcatcaatggcgcttccacgatggaagatcgtccgtcaacgcgcaactttttc |
| | | gaactgaatcgcggtgcaattaaatgtgaaggtctggatgtggaagatctgctgtcctattatgtcgtgtatgtgttctctaaacgc |
| | | taagcgaaaaatcaataaggaggcaacaagatgtgcgaaaaacatcttaatcatgcgagggatggtttctaatggatgaaatc |
| | | gtcaaaaatatccgcgaaggcacgcacgtcctgctgccgttctatgaaaccctgccggaactgaatctgtcactgggcaaatc |
| | | tccgctgccgagtctggaatatggtgcaaactactttctgcagatttctcgtgtgaacgatctgaatcgcatgccgaccgacatg |
| | | ctgaaactgttcacgcatgatatcatgctgccggaaagcgatctggacaaagtctacgaaatcctgaaaatcaactccgttaaat |
| | | actacggccgttcaaccaaagcggatgccgtggttgcagacctgtccgctcgcaataaactgtttaaacgtgaacgcgatgct |
| | | attaaatcgaacaatcacctgaccgaaaacaacctgtacatcagcgattacaaaatgctgacgtttgacgtgttccgtccgctgt |
| | | tcgatttcgttaacgaaaaatactgcatcatcaaactgccgaccctgtttggccgtggtgtgattgatacgatgcgcatctactgc |
| | | agcctgttcaaaaatgtccgcctgctgaaatgtgtgtcggatagctggctgaaagactctgcgattatggtggccagtgacgttt |
| | | gtaagaaaaacctggacctgtttatgtcccatgtcaaatcagtgaccaaaagctctagttggaaagacgttaattcggtccaattt |
| | | agcattctgaacaatccggttgatacggaattcatcaacaaattcctggaattctctaaccgtgtttacgaagcactgtattacgtc |
| | | cacagtctgctgtactcctcaatgaccteggactccaaatccatcgaaaataaacatcaacgccgcctggtgaaactgctgctg |
| | | gggagcgcttggagccacccgcagttcgaaaaaggtggaggttctggcggtggatcgggaggttcagegtggagccaccc |
| | | gcagttcgagaaataaccaggcatcaaataaaacgaaaggctcagtcgaaagactgggcctttcgttttatctgttgtttgtcgg |
| | | tgaacgctctctactagagtcacactggctcaccttcggggggcctttctgcgtttata |
| 52 | Combination | tcgacatttatcccttgcggcgaatacttacagccagcgaaaaatcaataaggaggcaacaagatgtgcgaaaaacatcttaat |
| | of genetic | catgcggtggagggtttctaatgaaacatcaccatcaccatcaccccatgagcgattacgacatccccactactgagaatcttta |
| | elements | ttttcagggcgccgacgctaatgtcgtgtcttcttctaccatcgcaacctatattgacgctctggcaaaaaacgcctcggaactg |
| | expressed in | gaacaacgctcaaccgcgtatgaaatcaacaatgaactggaactggtgtttatcaaaccgccgctgattacgctgaccaacgt |
| | strain 815934 | ggttaatatcagcaccattcaggaatcttttattcgtttcacggttaccaacaaagaaggcgtcaaaatccgcacgaaaattccg |
| | (Promoter | ctgagcaaagttcatggtctggatgtgaaaaacgttcaactggtcgacgcaatcgataatattgtgtgggaaaagaaaagcctg |
| | (apFAB124); | gttaccgaaaatcgtctgcataaagaatgcctgctgcgtctgagcacggaagaacgccacatctttctggactataaaaaatac |
| | RBS | ggcagctctatccgcctggaactggtgaacctgatccaggctaaaaccaaaaacttcacgatcgatttcaaactgaaatattttc |
| | (BCDRBS_alt1_ | tgggcagtggtgctcaatccaaaagttccctgctgcatgcgatcaaccacccgaaaagtgtccgaatacctccctggaaatt |
| | BD14); His- | gaattcaccccgcgcgacaacgaaacggtgccgtacgatgaactgattaaagaactgaccacgctgtcacgtcatatctttatg |
| | Tag; D1 (E. | gcgtcgccggaaaacgttattctgagcccgccgatcaatgccccgattaaaaccttcatgctgccgaaacaggacattgttgg |
| | coli recode 1); | cctggatctggaaaacctgtatgcggtcacgaaaaccgatggtattccgatcaccattcgcgtgacgtcgaatggcctgtattg |
| | RBS | ctactttacccacctgggttatattatccgttacccggttaaacgcattatcgactccgaagtcgtggttttcggcgaagcggtca |
| | (BCDRBS_alt1_ | aagataaaaattggaccgtgtatctgatcaaactgattgaaccggtgaacgccatcaacgatcgtctggaagaatcaaaatacg |
| | BD15); D12 | tggaatcgaaactggttgacatctgtgatcgcatcgttttcaaaagcaaaaaatacgaaggtccgttcaccacgacctctgaagt |
| | (E. coli recode | cgtggatatgctgagtacctatctgccgaaacagccggaaggcgtgatcctgttttacagcaaaggtccgaaatctaacatcga |
| | 1); Twin Strep | cttcaaaatcaaaaaagaaaacaccatcgatcaaacggccaatgttgtctttcgttatatgtcatcggaaccgattatctttggcg |
| | Tag; | aaagctctatcttcgtggaatacaaaaaattctcgaacgataaaggcttcccgaaagaatacggcagcggtaaaattgtcctgt |
| | Terminator | ataacggtgtgaattacctgaacaatatctattgcctggaatacattaacacccataatgaagttggcattaaatctgtggttgtcc |
| | ((BBa_B0015 | cgatcaaatttattgcagaattcctggtcaacggtgaaatcctgaaaccgcgtattgacaaaaccatgaaatacatcaacagtga |
| | (Double | agattactacggtaaccagcataacatcatcgtggaacacctgegcgaccaatctatcaaaatcggcgatatcttcaacgaaga |
| | Terminator | caaactgagtgatgtcggtcaccagtatgcgaacaatgataaatttcgtctgaacccggaagtgtcctacttcaccaataaacgt |
| | B0010, | acgcgcggcccgctgggtatcctgtcaaattatgtcaaaaccctgctgatttcaatgtactgttcgaaaacgtttctggatgacag |
| | B0012)) | caacaaacgcaaagttctggccattgactttggcaatggtgcagatctggaaaaatatttctacggcgaaatcgctctgctggtt |
| | | gcgaccgatccggacgcggatgccattgcacgtggcaacgaacgctataacaaactgaattctggtatcaaaaccaaatact |
| | | acaaattcgactacatccaggaaaccattcgtagtgatacgttcgtgagttccgttcgcgaagtcttttatttcggcaaattcaaca |
| | | tcatcgattggcaattcgccatccattattctttccatccgcgtcactacgcaaccgtgatgaacaatctgagtgaactgacggct |
| | | tccggcggtaaagttctgattacgacgatggatggtgataaactgtccaaactgaccgataagaaaaccttcattatccacaaa |
| | | aacctgccgtcatcggaaaactacatgtcagtggaaaaaatcgccgatgaccgcattgtggtttataacccgagcacgatgtct |
| | | accccgatgacggaatacatcattaagaaaaacgatatcgtccgtgtgtttaatgaatacggtttcgttctggtcgacaacgttga |
| | | ttttgcaaccattatcgaacgcagcaaaaaattcatcaatggcgcttccacgatggaagatcgtccgtcaacgcgcaactttttc |
| | | gaactgaatcgcggtgcaattaaatgtgaaggtctggatgtggaagatctgctgtcctattatgtcgtgtatgtgttctctaaacgc |
| | | taaaataattttgtttaactttaagaaggaggtatatccatggctagcatgactaaacatcttaatcatgcgggggagtctttctaat |
| | | ggatgaaatcgtcaaaaatatccgcgaaggcacgcacgtcctgctgccgttctatgaaaccctgccggaactgaatctgtcac |
| | | tgggcaaatctccgctgccgagtctggaatatggtgcaaactactttctgcagatttctcgtgtgaacgatctgaatcgcatgcc |
| | | gaccgacatgctgaaactgttcacgcatgatatcatgctgccggaaagcgatctggacaaagtctacgaaatcctgaaaatca |
| | | actccgttaaatactacggccgttcaaccaaagcggatgccgtggttgcagacctgtccgctcgcaataaactgtttaaacgtg |
| | | aacgcgatgctattaaatcgaacaatcacctgaccgaaaacaacctgtacatcagcgattacaaaatgctgacgtttgacgtgtt |
| | | ccgtccgctgttcgatttcgttaacgaaaaatactgcatcatcaaactgccgaccctgtttggccgtggtgtgattgatacgatgc |
| | | gcatctactgcagcctgttcaaaaatgtccgcctgctgaaatgtgtgtcggatagctggctgaaagactctgcgattatggtggc |
| | | cagtgacgtttgtaagaaaaacctggacctgtttatgtcccatgtcaaatcagtgaccaaaagctctagttggaaagacgttaatt |
| | | cggtccaatttagcattctgaacaatccggttgatacggaattcatcaacaaattcctggaattctctaaccgtgtttacgaagcac |
| | | tgtattacgtccacagtctgctgtactcctcaatgacctcggactccaaatccatcgaaaataaacatcaacgccgcctggtgaa |
| | | actgctgctggggagcgcttggagccacccgcagttcgaaaaaggtggaggttctggcggtggatcgggaggttcagcgtg |
| | | gagccacccgcagttcgagaaataaccaggcatcaaataaaacgaaaggctcagtcgaaagactgggcctttcgttttatctg |
| | | ttgtttgtcggtgaacgctctctactagagtcacactggctcaccttcgggtgggcctttctgcgtttata |
| 53 | Combination | ttccctattaatcatccggctcgtataatgtgtggagcgaaaaatcaataaggaggcaacaagatgtgcgaaaaacatcttaatc |
| | of genetic | atgcggtggagggtttctaatgaaacatcaccatcaccatcaccccatgagegattacgacatccccactactgagaatctttat |
| | elements | tttcagggcgccgacgccaacgtagtgagctcgtccacgattgctacatacatcgacgcactggctaaaaacgcgagtgaatt |
| | expressed in | agagcaacgttcaaccgcctatgaaatcaacaacgaacttgagctcgtctttattaagcctccgctaatcaccctgactaacgtt |
| | strain 816019 | gttaatatatctaccatccaggaaagcttcattcgcttcactgttactaacaaagaaggcgtaaaaatcaggactaaaatcccatt |
| | (Promoter | gtctaaggtgcacgggctggatgtgaaaaacgttcagctggttgacgctattgacaacatcgtatgggaaaagaaatccctcgt |
| | (apFAB277); | aaccgaaaaccgtctgcataaagaatgtctgctgcgtctgagcacggaggaacgacacatctttctggattacaaaaaatatg |
| | RBS | gtagttctattcgtctggagctggtgaacctgatccaggcaaagaccaaaaatttcacaattgacttcaaactaaaatactttctg |
| | (BCDRBS_alt1_ | ggctccggtgcgcagagcaaatcttccctgttgcatgctatcaaccacccgaaaagccgcccgaatacttctctggaaatcga |
| | BD14); His- | gttcaccccccgcgataacgaaactgtcccatacgatgagcttattaaggaactgaccacgctgtcccgtcacatttttatggcg |
| | Tag; D1 (E. | agcccggaaaacgttatattatcgccgcctatcaacgctccgatcaagaccttcatgttgccgaaacaagacatcgtcggtctg |
| | coli recode | gatctggagaacctgtacgcagttactaaaaccgacggcatccccatcactatcagagtaacgtcaaacggattgtattgctatt |
| | 12); RBS | tcacccatctgggttacattattcgttacccggtgaaacgcatcatagattctgaagttgttgttttcggcgaagccgtaaaggac |
| | (BCDRBS_alt1_ | aaaaactggaccgtctatctgatcaagctaatcgaaccggttaatgctatcaacgatcggctggaagaatcgaaatacgtagaa |
| | BD15); D12 | tctaaactggtggatatttgcgaccgtattgtctttaaatcgaaaaagtacgagggtcctttcactactactagcgaagtcgtgga |
| | (E. coli recode | catgctctctacgtacctgccgaaacagcctgagggcgttatcctgttctatagcaaaggtccgaaatccaacatcgattttaag |
| | 2); Twin Strep | attaaaaaggaaaacaccattgatcagacggctaatgtagttttccggtacatgtctagcgagccgatcatctttggcgaatcttc |
| | Tag; | tatctttgtagaatataaaaagttcagcaacgacaaaggattcccaaaagaatacgggtccgggaaaatcgtcttatacaacgg |
| | Terminator | tgttaactacttgaacaacatctattgcctggaatatatcaatactcacaatgaagttggtattaaatcagtggttgttccgataaaat |
| | ((BBa_B0015 | tcatcgcggaatttctggtcaatggcgaaatcctgaaaccccgcattgataagaccatgaaatacataaactccgaagactact |
| | (Double | acggtaaccagcataacatcatcgtggaacacctgagagatcagagtatcaaaatcggcgacattttcaatgaggacaagtta |
| | Terminator | agcgacgtgggccatcaatacgcaaacaacgacaaattccgtctgaacccggaggtttcctatttcaccaacaaacgtacccg |
| | B0010, | aggtccgcttggcatcctctccaattacgtaaaaaccctgctgatttctatgtattgttcaaaaacgttcctggatgacagcaacaa |
| | B0012)) | aaggaaggtactggctatcgatttcggtaacggcgcggatctggaaaagtacttttacggtgaaatcgctctgttagtcgcaact |
| | | gatccggacgccgacgcaattgctcgcggaaatgaacgttacaacaaactgaactccggtattaaaacaaagtattataaattc |
| | | gactatatccaggagactatccgctctgatactttcgtgagcagcgtgcgtgaggttttttactttggtaaattcaacattattgact |
| | | ggcagtttgcgatccactacagctttcacccgcgtcactatgcgaccgttatgaataacctatcggaactcacggctagcggcg |
| | | gcaaagtgctgattactactatggacggtgacaaactgtctaagctgaccgataagaaaaccttcatcatccacaaaaacttgc |
| | | caagttctgagaactatatgtctgttgaaaaaattgcggacgaccgcatcgtcgtttacaacccatctaccatgtccacccctatg |
| | | acagagtacatcatcaaaaagaacgacatagttcgtgttttcaacgaatacggcttcgtactggtagataacgtcgattttgctac |
| | | cattatcgagcgttcgaaaaaattcattaacggtgcttccactatggaagatcgtccgtccactcgtaacttttttgaattaaaccgt |
| | | ggcgcaatcaaatgcgaagggctggatgtggaagacctcctgtcttactacgttgtatacgtcttctctaaacgctaaaataattt |
| | | tgtttaactttaagaaggaggtatatccatggctagcatgactaaacatcttaatcatgcgggggagtctttctaatggatgagatc |
| | | gttaagaacattcgtgaaggtacgcatgtgcttttgccattttacgaaactctcccggaactgaatctgtccttaggcaaaagccc |
| | | tctaccctctctggagtatggggccaactacttcctgcaaatctcacgcgtcaacgacctgaatcgaatgccgaccgacatgct |
| | | gaaactgttcactcacgatataatgctgccggaaagtgatctggacaaagtatatgaaatcctgaaaatcaacagcgttaagta |
| | | ctacggacggtcgaccaaagcggacgctgttgtagcagatctgtctgctcgcaacaaactctttaaacgtgaacgtgacgctat |
| | | taagtccaacaaccacctgacagagaacaatctctatatctctgactacaaaatgttgactttcgatgtgttccgtccgctgtttga |
| | | tttcgtgaacgaaaaatattgcattatcaaactgccgaccctgttcggccgtggtgttattgacaccatgcgcatctactgtagcct |
| | | cttcaagaatgtcagactactgaaatgcgtgtccgatagctggctgaaagacagcgcaatcatggtagcctcagacgtttgcaa |
| | | aaagaacctggatctgtttatgtcccatgttaaatccgttactaagtctagctcgtggaaagatgttaacagcgtacagttttctattt |
| | | tgaacaaccctgttgacacggaatttatcaacaaattcctggagttctctaaccgtgtatacgaagcgctgtattacgtgcactcc |
| | | ttactgtactcttctatgaccagcgatagtaagtctatcgaaaataaacaccagcgccgtctggtaaaactgctccttgggagcg |
| | | cttggagccacccgcagttcgaaaaaggtggaggttctggcggtggatcgggaggttcagcgtggagccacccgcagttcg |
| | | agaaataaccaggcatcaaataaaacgaaaggctcagtcgaaagactgggcctttcgttttatctgttgtttgtcggtgaacgct |
| | | ctctactagagtcacactggctcaccttcgggtgggcctttctgcgtttata |
| 54 | Combination | ttccctattaatcatccggctcgtataatgtgtggagcgaaaaatcaataaggaggcaacaagatgtgcgaaaaacatcttaatc |
| | of genetic | atgcggtggagggtttctaatgaaacatcaccatcaccatcaccccatgagcgattacgacatccccactactgagaatctttat |
| | elements | tttcagggcgccgacgccaacgtagtgagctcgtccacgattgctacatacatcgacgcactggctaaaaacgcgagtgaatt |
| | expressed in | agagcaacgttcaaccgcctatgaaatcaacaacgaacttgagctcgtctttattaagcctccgctaatcaccctgactaacgtt |
| | strain 816020 | gttaatatatctaccatccaggaaagcttcattcgcttcactgttactaacaaagaaggcgtaaaaatcaggactaaaatcccatt |
| | (Promoter | gtctaaggtgcacgggctggatgtgaaaaacgttcagctggttgacgctattgacaacatcgtatgggaaaagaaatccctcgt |
| | (apFAB277); | aaccgaaaaccgtctgcataaagaatgtctgctgcgtctgagcacggaggaacgacacatctttctggattacaaaaaatatg |
| | RBS | gtagttctattcgtctggagctggtgaacctgatccaggcaaagaccaaaaatttcacaattgacttcaaactaaaatactttctg |
| | (BCDRBS_alt1_ | ggctccggtgcgcagagcaaatcttccctgttgcatgctatcaaccacccgaaaagccgcccgaatacttctctggaaatcga |
| | BD14); His- | gttcaccccccgcgataacgaaactgtcccatacgatgagcttattaaggaactgaccacgctgtcccgtcacatttttatggcg |
| | Tag; D1 (E. | agcccggaaaacgttatattatcgccgcctatcaacgctccgatcaagaccttcatgttgccgaaacaagacategtcggtctg |
| | coli recode | gatctggagaacctgtacgcagttactaaaaccgacggcatccccatcactatcagagtaacgtcaaacggattgtattgctatt |
| | 12); RBS | tcacccatctgggttacattattcgttacccggtgaaacgcatcatagattctgaagttgttgttttcggcgaagccgtaaaggac |
| | (BCDRBS_alt1_ | aaaaactggaccgtctatctgatcaagctaatcgaaccggttaatgctatcaacgatcggctggaagaatcgaaatacgtagaa |
| | BD21); D12 | tctaaactggtggatatttgcgaccgtattgtctttaaatcgaaaaagtacgagggtcctttcactactactagcgaagtcgtgga |
| | (E. coli recode | catgctctctacgtacctgccgaaacagcctgagggcgttatcctgttctatagcaaaggtccgaaatccaacatcgattttaag |
| | 2); Twin Strep | attaaaaaggaaaacaccattgatcagacggctaatgtagttttccggtacatgtctagcgagccgatcatctttggcgaatcttc |
| | Tag; | tatctttgtagaatataaaaagttcagcaacgacaaaggattcccaaaagaatacgggtccgggaaaatcgtcttatacaacgg |
| | Terminator | tgttaactacttgaacaacatctattgcctggaatatatcaatactcacaatgaagttggtattaaatcagtggttgttccgataaaat |
| | ((BBa_B0015 | tcatcgcggaatttctggtcaatggcgaaatcctgaaaccccgcattgataagaccatgaaatacataaactccgaagactact |
| | (Double | acggtaaccagcataacatcatcgtggaacacctgagagatcagagtatcaaaatcggcgacattttcaatgaggacaagtta |
| | Terminator | agcgacgtgggccatcaatacgcaaacaacgacaaattccgtctgaacccggaggtttcctatttcaccaacaaacgtacccg |
| | B0010, | aggtccgcttggcatcctctccaattacgtaaaaaccctgctgatttctatgtattgttcaaaaacgttcctggatgacagcaacaa |
| | B0012)) | aaggaaggtactggctatcgatttcggtaacggcgcggatctggaaaagtacttttacggtgaaatcgctctgttagtcgcaact |
| | | gatccggacgccgacgcaattgctcgcggaaatgaacgttacaacaaactgaactccggtattaaaacaaagtattataaattc |
| | | gactatatccaggagactatccgctctgatactttcgtgagcagcgtgcgtgaggttttttactttggtaaattcaacattattgact |
| | | ggcagtttgcgatccactacagctttcacccgcgtcactatgcgaccgttatgaataacctatcggaactcacggctagcggcg |
| | | gcaaagtgctgattactactatggacggtgacaaactgtctaagctgaccgataagaaaaccttcatcatccacaaaaacttgc |
| | | caagttctgagaactatatgtctgttgaaaaaattgcggacgaccgcatcgtcgtttacaacccatctaccatgtccacccctatg |
| | | acagagtacatcatcaaaaagaacgacatagttcgtgttttcaacgaatacggcttcgtactggtagataacgtcgattttgctac |
| | | cattatcgagcgttcgaaaaaattcattaacggtgcttccactatggaagatcgtccgtccactcgtaacttttttgaattaaaccgt |
| | | ggcgcaatcaaatgcgaagggctggatgtggaagacctcctgtcttactacgttgtatacgtcttctctaaacgctaagcgaaa |
| | | aatcaataaggaggcaacaagatgtgcgaaaaacatcttaatcatgcgagggatggtttctaatggatgagatcgttaagaaca |
| | | ttcgtgaaggtacgcatgtgcttttgccattttacgaaactctcccggaactgaatctgtccttaggcaaaagccctctaccctetc |
| | | tggagtatggggccaactacttcctgcaaatctcacgcgtcaacgacctgaatcgaatgccgaccgacatgctgaaactgttc |
| | | actcacgatataatgctgccggaaagtgatctggacaaagtatatgaaatcctgaaaatcaacagcgttaagtactacggacg |
| | | gtcgaccaaagcggacgctgttgtagcagatctgtctgctcgcaacaaactctttaaacgtgaacgtgacgctattaagtccaa |
| | | caaccacctgacagagaacaatctctatatctctgactacaaaatgttgactttcgatgtgttccgtccgctgtttgatttcgtgaac |
| | | gaaaaatattgcattatcaaactgccgaccctgttcggccgtggtgttattgacaccatgcgcatctactgtagcctcttcaagaa |
| | | tgtcagactactgaaatgcgtgtccgatagctggctgaaagacagcgcaatcatggtagcctcagacgtttgcaaaaagaacc |
| | | tggatctgtttatgtcccatgttaaatccgttactaagtctagctcgtggaaagatgttaacagegtacagttttctattttgaacaac |
| | | cctgttgacacggaatttatcaacaaattcctggagttctctaaccgtgtatacgaagcgctgtattacgtgcactccttactgtact |
| | | cttctatgaccagcgatagtaagtctatcgaaaaaaacaccagcgccgtctggtaaaactgctccttgggagcgcttggagcc |
| | | acccgcagttcgaaaaaggtggaggttctggcggtggatcgggaggttcagcgtggagccacccgcagttcgagaaataac |
| | | caggcatcaaataaaacgaaaggctcagtcgaaagactgggcctttcgttttatctgttgtttgtcggtgaacgctctctactaga |
| | | gtcacactggctcaccttcggggggcctttctgcgtttata |

### EQUIVALENTS

Those skilled in the art will recognize, or be able to ascertain using no more than routine experimentation, many equivalents to the specific embodiments of the invention described here. Such equivalents are intended to be encompassed by the following claims.

All references, including patent documents, are incorporated by reference in their entirety.

It should be appreciated that sequences disclosed in this application may or may not contain secretion signals. The sequences disclosed in this application encompass versions with or without secretion signals. It should also be understood that protein sequences disclosed in this application may be depicted with or without a start codon (M). The sequences disclosed in this application encompass versions with or without start codons. Accordingly, in some instances amino acid numbering may correspond to protein sequences containing a start codon, while in other instances, amino acid numbering may correspond to protein sequences that do not contain a start codon. It should also be understood that sequences disclosed in this application may be depicted with or without a stop codon. The sequences disclosed in this application encompass versions with or without stop codons. Aspects of the disclosure encompass host cells comprising any of the sequences described in this application and fragments thereof.

1. A non-naturally occurring nucleic acid comprising:
a) a promoter, wherein the promoter comprises a sequence that is at least 90% identical to SEQ ID NO: 8 or 9; and
b) a nucleic acid encoding an amino acid sequence that is at least 90% identical to SEQ ID NO: 6 or 29, and/or a nucleic acid encoding an amino acid sequence that is at least 90% identical to SEQ ID NO: 7 or 31,
wherein (a) and (b) are operably linked, and wherein the non-naturally occurring nucleic acid further comprises a ribosome binding site (RBS).

2. The non-naturally occurring nucleic acid of claim 1, wherein the promoter is inducible by lactose and/or galactose.

3. The non-naturally occurring nucleic acid of claim 1 or 2, wherein the non-naturally occurring nucleic acid further comprises a terminator.

4. The non-naturally occurring nucleic acid of any one of claims 1-3, wherein:
a) the RBS comprises a sequence that is at least 90% identical to SEQ ID NO: 10, 11, 12, 13, 14, 15, 16, 17, 37, 38, or 45 and/or b) the terminator comprises a sequence that is at least 90% identical to SEQ ID NO: 18, 19, or 20.

5. The non-naturally occurring nucleic acid of any one of claims 1-4, wherein:
a) the nucleic acid encoding the amino acid sequence that is at least 90% identical to SEQ ID NO: 6 or 29 comprises a nucleic acid sequence that is at least 90% identical to SEQ ID NO: 2, 3, 33 or 34; and/or
b) the nucleic acid encoding the amino acid sequence that is at least 90% identical to SEQ ID NO: 7 or 31 comprises a nucleic acid sequence that is at least 90% identical to SEQ ID NO: 4, 5, 35 or 36.

6. The non-naturally occurring nucleic acid of any one of claims 3-5, wherein the promoter, RBS, and terminator are operably linked to the nucleic acid of claim 1(b).

7. The non-naturally occurring nucleic acid of any one of claims 1-6 wherein the nucleic acid in claim 1(b) encodes the amino acid sequence of SEQ ID NO: 6 or 29.

8. The non-naturally occurring nucleic acid of any one of claims 1-6, wherein the nucleic acid in claim 1(b) encodes the amino acid sequence of SEQ ID NO: 7 or 31.

9. The non-naturally occurring nucleic acid of any one of claims 1-6, wherein the nucleic acid in claim 1(b) encodes the amino acid sequence of SEQ ID NO: 6 or 29 and also encodes the amino acid sequence of SEQ ID NO: 7 or 31.

10. A non-naturally occurring nucleic acid comprising:
a) a first promoter, wherein the first promoter comprises a sequence that is at least 90% identical to SEQ ID NO: 8 or 9;
b) a first nucleic acid, wherein the first nucleic acid encodes an amino acid sequence that is at least 90% identical to SEQ ID NO: 6 or 29;
c) a second promoter, wherein the second promoter comprises a sequence that is at least 90% identical to SEQ ID NO: 8 or 9; and
d) a second nucleic acid, wherein the second nucleic acid encodes an amino acid sequence that is at least 90% identical to SEQ ID NO: 7 or 31,
wherein (a) and (b) are operably linked, and wherein (c) and (d) are operably linked, and wherein the non-naturally occurring nucleic acid further comprises at least one ribosome binding site (RBS).

11. The non-naturally occurring nucleic acid of claim 10, wherein the first promoter and/or the second promoter is inducible by lactose and/or galactose.

12. The non-naturally occurring nucleic acid of claim 10 or 11, wherein the non-naturally occurring nucleic acid further comprises at least one terminator.

13. The non-naturally occurring nucleic acid of any one of claims 10-12, wherein:
a) the RBS comprises a sequence that is at least 90% identical to SEQ ID NO: 10, 11, 12, 13, 14, 15, 16, 17, 37, 38, or 45 and/or
b) the terminator comprises a sequence that is at least 90% identical to SEQ ID NO: 18, 19, or 20.

14. The non-naturally occurring nucleic acid of any one of claims 10-13, wherein:
a) the first nucleic acid comprises a sequence that is at least 90% identical to SEQ ID NO: 2, 3, 33 or 34; and/or
b) the second nucleic acid comprises a sequence that is at least 90% identical to SEQ ID NO: 4, 5, 35 or 36.

15. The non-naturally occurring nucleic acid of any one of claims 10-14, wherein the non-naturally occurring nucleic acid comprises a sequence that is at least 90% identical to any one of SEQ ID NO: 21-28, or 49-54.

16. A non-naturally occurring nucleic acid comprising a sequence that is at least 90% identical to any one of SEQ ID NOs: 21-28, or 49-54.

17. The non-naturally occurring nucleic acid of any one of claims 1-16, wherein the non-naturally occurring nucleic acid does not encode a fusion protein.

18. A host cell comprising the non-naturally occurring nucleic acid of any one of claims 1-17.

19. The host cell of claim 18, wherein the non-naturally occurring nucleic acid is integrated into the genome of the host cell in whole or in part.

20. A host cell comprising one or more non-naturally occurring nucleic acids comprising:
a promoter, wherein the promoter comprises a sequence that is at least 90% identical to SEQ ID NO: 8 or 9, and
a nucleic acid encoding an amino acid sequence that is at least 90% identical to SEQ ID NO: 6 or 29 and/or a nucleic acid encoding an amino acid sequence that is at least 90% identical to SEQ ID NO: 7 or 31,
wherein one or more of the non-naturally occurring nucleic acids further comprise a ribosome binding site (RBS).

21. The host cell of claim 20, wherein the promoter is inducible by lactose and/or galactose.

22. The host cell of claim 21, wherein the RBS comprises a sequence that is at least 90% identical to one of SEQ ID NOs: 10-17, 37, 38, or 45.

23. The host cell of any one of claims 19-22, wherein one or more of the non-naturally occurring nucleic acids further comprises a terminator.

24. The host cell of any one of claims 19-23, wherein one or more of the non-naturally occurring nucleic acids is integrated into the genome of the host cell.

25. The host cell of any one of claims 19-23, wherein one or more of the non-naturally occurring nucleic acids is expressed on a plasmid.

26. The host cell of any one of claims 19-25, wherein the host cell is a bacterial cell.

27. The host cell of claim 26, wherein the bacterial cell is an E. coli cell.

28. The host cell of any one of claims 19-27 wherein one or more of the nucleic acid sequences encodes an amino acid sequence of SEQ ID NO: 6 or 29.

29. The host cell of any one of claims 19-27, wherein one or more of the nucleic acid sequences encodes an amino acid sequence of SEQ ID NO: 7 or 31.

30. The host cell of any one of claims 19-27, wherein one or more of the nucleic acids encodes an amino acid sequence of SEQ ID NO: 6 or 29 and also encodes an amino acid sequence of SEQ ID NO: 7 or 31.

31. A host cell comprising one or more non-naturally occurring nucleic acids comprising:
a) a first promoter, wherein the first promoter comprises a sequence that is at least 90% identical to SEQ ID NO: 8 or 9;
b) a first nucleic acid, wherein the first nucleic acid encodes an amino acid sequence that is at least 90% identical to SEQ ID NO: 6 or 29;
c) a second promoter, wherein the second promoter comprises a sequence that is at least 90% identical to SEQ ID NO: 8 or 9; and
d) a second nucleic acid, wherein the second nucleic acid encodes an amino acid sequence that is at least 90% identical to SEQ ID NO: 7 or 31,
wherein (a) and (b) are operably linked, and wherein (c) and (d) are operably linked, and wherein one or more of the non-naturally occurring nucleic acids further comprises at least one ribosome binding site (RBS).

32. The host cell of claim 31, wherein the promoter is inducible by lactose and/or galactose.

33. The host cell of claim 31 or 32, wherein one or more of the non-naturally occurring nucleic acids further comprises at least one terminator.

34. The host cell of claim 32 or 33, wherein:
a) the RBS comprises a sequence that is at least 90% identical to SEQ ID NO: 10, 11, 12, 13, 14, 15, 16, 17, 37, 38, or 45 and/or
b) the terminator comprises a sequence that is at least 90% identical to SEQ ID NO: 18, 19, or 20.

35. The host cell of any one of claims 31-34, wherein:
a) the first nucleic acid comprises a sequence that is at least 90% identical to SEQ ID NO: 2, 3, 33 or 34; and/or
b) the second nucleic acid comprises a sequence that is at least 90% identical to SEQ ID NO: 4, 5, 35 or 36.

36. The host cell of any one of claims 31-35, wherein one or more of the non-naturally occurring nucleic acids comprises a sequence that is at least 90% identical to any one of SEQ ID NO: 21-28, or 49-54.

37. The host cell of any one of claims 18-36, wherein the host cell is capable of producing at least 1-fold, 2-fold, 3-fold, 4-fold or 5-fold more vaccinia capping enzyme as compared to a control host cell, wherein the control host cell is a wildtype E. coli cell.

38. The host cell of any one of claims 18-37, wherein the host cell is capable of producing at least 50 mg/L, 100 mg/L, 150 mg/L, 200 mg/L, 250 mg/L, 300 mg/L, 350 mg/L, 400 mg/L, or 450 mg/L vaccinia capping enzyme.

39. The host cell of any one of claims 18-38, wherein the non-naturally occurring nucleic acid does not encode a fusion protein.

40. A method of producing vaccinia capping enzyme comprising culturing the host cell of any one of claims 18-39.

41. The method of claim 40, wherein the method further comprises purification of the vaccinia capping enzyme.

42. A non-naturally occurring nucleic acid comprising:
(a) a promoter, wherein the promoter is a Ptac promoter or a functional fragment thereof, or a P(T5) 2xlacO promoter or a functional fragment thereof; and
(b) a nucleic acid encoding a D1 subunit of VCE and/or a D12 subunit of vaccinia capping enzyme,
wherein (a) and (b) are operably linked, and wherein the non-naturally occurring nucleic acid further comprises a ribosome binding site (RBS).

43. The non-naturally occurring nucleic acid of claim 42, wherein the promoter is inducible by lactose and/or galactose.

44. The non-naturally occurring nucleic acid of claim 42 or 43, wherein the non-naturally occurring nucleic acid does not encode a fusion protein.
